(19) United States
(12) Patent Application Publication
(10) Pub. No.: US 2002/0081688 A1
(43) Pub. Date: Jun. 27, 2002

(54) Retinoid pathway assays, and compositions therefrom

(76) Inventors: Carl Alexander Kamb, Salt Lake City, UT (US); Burt Timothy Richards, Midway, UT (US); Jon Karpilow, Boulder, CO (US)

Correspondence Address: Joseph A. Williams, Jr., MARSHALL, GERSTEIN, MURRAY & BORUN, 6300 Sears Tower, 233 South Wacker Drive, Chicago, IL, 60606-6402, US

(21) Appl. No.: 09/990,747

(22) Filed: Nov. 16, 2001

Related U.S. Application Data
(63) Continuation-in-part of application No. 08/812,994, filed on Mar. 4, 1997, now Pat. No. 5,955,275.
(60) Provisional application No. 60/249,468, filed on Nov. 17, 2000.

(51) Int. Cl. C12Q 1/68; C12N 9/02; C12P 21/02; C12N 5/06

(52) U.S. Cl. 435/189; 435/6; 435/69.1; 435/320.1; 435/325

(57) ABSTRACT

Methods for assaying a cellular pathway, and more particularly a retinoic acid-related pathway, are disclosed. The assays of the invention utilize particular host cells with desired retinoic acid pathway elements, and results in the identification of biologically active phenotypic probes and cellular targets and fragments, variants and mimetics thereof.

This application claims priority from, and is a continuation-in-part of, 08/812,994, now issued as U.S. Pat. No. 5,955,275, and U.S. application Ser. No. 60/249,768 (VEN007/00/P1, filed Nov. 17, 2000), the entire disclosures of which are specifically incorporated by reference herein in their entireties.

### FIELD OF THE INVENTION

The present invention relates to certain nucleic acid sequences, amino acid sequences, other compositions and methods relating to the characterization and physiologic implications of the retinoic acid pathway.

### BACKGROUND OF THE INVENTION

The use of retinoids in the treatment of disease has expanded in the past decade. Retinoid therapy has been found to be helpful in the treatment of multiple forms of cancer including (i) small and non-small cell lung carcinoma (Ruotsalainen, T. et al. (2000), “Interferon-alpha and 13-cis-retinoic acid as maintenance therapy after high-dose combination chemotherapy with growth factor support for small cell lung cancer—a feasibility study,” Anticancer Drugs 11(2):101-8; Recchia, F. et al. (1999), “Carboplatin, vindesine, 5-fluorouracil-leucovorin and 13-cis retinoic acid in the treatment of advanced non-small cell lung cancer. A phase II study.” Clin Ter 150(4):269-74.); (ii) breast cancer (Wang, Q. et al. (2000) “1,25-Dihydroxyvitamin D3 and all-trans-retinoic acid sensitize breast cancer cells to chemotherapy-induced cell death.” Cancer Res 60(7):2040-8); (iii) pancreatic cancer (Riecken, E. O. et al. (1999) “Retinoids in pancreatic cancer.” Ann Oncol 10 Suppl 4:197-200); (iv) Karposi's sarcoma (Yarchoan, R. (1999) “Therapy for Kaposi's sarcoma: recent advances and experimental approaches.” J Acquir Immune Defic Syndr 21 Suppl 1:S66-73); (v) neuroblastoma (Matthay, K. K. et al. (1999) “Treatment of high-risk neuroblastoma with intensive chemotherapy, radiotherapy, autologous bone marrow transplantation, and 13-cis-retinoic acid” New Engl J Med 341(16):1165-73); (vi) renal cancer (Berg, W. J. et al, (1999) “Up-regulation of retinoic acid receptor beta expression in renal cancers in vivo correlates with response to 13-cis-retinoic acid and interferon-alpha-2a.” Clin Cancer Res 5(7):1671-5); (vii) prostate cancer (Sporn, M. B. (1999) “New agents for chemoprevention of prostate cancer.” Eur Urol 35(5-6):420-3); and (viii) ovarian cancer (Srivastava, R. K. et al. (1999) “Synergistic effects of retinoic acid and 8-Cl-cAMP on apoptosis require caspase-3 activation in human ovarian cancer cells.” Oncogene 18(9):1755-63), as well as several other malignancies. In addition, retinoid therapy has also proven useful in treating several forms of dermatitis including but not limited to (i) hyperkeratosis (Okan, G. et al. (1999) “Nevoid hyperkeratosis of the nipple and areola: treatment with topical retinoic acid.” J Eur Acad Dermatol Venereol 13(3):218-20); (ii) eczema (Bollag, W. et al. (1999) “Successful treatment of chronic hand eczema with oral 9-cis-retinoic acid.” Dermatology 199(4):308-12)” (iii) Darier's disease (Burge, S. (1999) “Management of Darier's disease.” Clin Exp Dermatol 24(2):53-6; (iv) Reiter's disease (Benoldi, D. et al. (1984) “Reiter's disease: successful treatment of the skin manifestations with oral etretinate.” Acta Denn Venereol 64(4):352-4); and (v) psoriasis (Peters, B. P. et al. (2000) “Pathophysiology and treatment of psoriasis.” Am J Health Syst Pharm 57(7):645-59).

One disease that has received particular attention due to its sensitivity to retinoids is acute promyelocytic leukemia (APL). APL is brought about by the clonal expansion of myeloid cells that have lost the ability to differentiate into red blood cells. As a result, patients inflicted with the disorder suffer from a unique hemorrhagic syndrome and disseminated intravascular coagulation.

APL is brought about by a reciprocal chromosomal translocation (t (15:17) (q21-q11-22). Rabbitts, T. H. (1994) “Chromosomal translocations in human cancer.” Nature, 372:143-149. These breaks lead to two unrelated genes, a portion of the retinoic acid receptor alpha (RARα) and a second gene designated PML (promyelocytic leukemia), to be ligated together to form a novel fusion protein, PML-RARα (FIG. 1). Considerable effort has gone into understanding the structure and function of both the chimeric gene product and its constituent components. RARα encodes a member of the Type II steroid receptor/transcription factor family and mediates the RA signal at specific RA-responsive promoters/enhancers called RAREs (retinoic acid response element). Upon entering the cell by simple diffusion, retinoic acid binds to either the homodimeric retinoic acid receptor (RAR/RAR) or the heterodimeric RAR/RXR (retinoid X receptor) complex localized in the cytoplasm or nuclear compartment (FIG. 2). The association with the appropriate receptor complex, and subsequent binding of this complex to a RARE element, up-regulates transcription of the downstream gene(s). Hashimoto, Y. and Shudo, K. (1991) “Retinoids and their nuclear receptors.” Cell Biol Rev 25(3):209-30, 233-235.

Like RAR, PML is believed to mediate its function in the nucleus. PML-specific antibodies have shown that the PML protein is distributed in discreet, punctate nuclear bodies that co-localize with DAXX, a molecule that has been identified as a component of the apoptotic pathway. Torii, S. et al. (1999) “Human Daxx regulates Fas-induced apoptosis from nuclear PML oncogenic domains (PODs).” EMBO J. 18(21):6037-49. From this association it has been hypothesized that PML plays a critical role in the regulation of cell growth and tumor suppression.

Though the mechanism by which the PML-RARα chimera induces APL has yet to be unraveled, studies have been performed to understand the contribution that each half of the protein makes to the disease phenotype. Through introduction of point mutations into the chimeric protein it has been determined that both the coiled-coil region of PML and the DNA-binding domain of RARα are necessary for the fusion protein to block myeloid differentiation. In contrast, analysis of the in vivo dimerization properties of the PML/RARα mutants has revealed that PML/RARα-PML and PML/RARα-RXR heterodimers are not necessary for PML/RARα activity on differentiation. Grignani, F. et al. (1996) “Effects on differentiation by the promyelocytic leukemia PML/RARα protein depend on the fusion of the PML protein dimerization and RARα DNA binding domains.” The EMBO Journal. 15:4949-4958.

Patients suffering from APL are currently treated with large doses of ATRA (all-trans retinoic acid), a procedure that does not kill cells by cytotoxic means but instead induces terminal differentiation of the APL blast cells (Chomienne, C. et al. (1996), “Retinoid differentiation therapy in promyelocytic leukemia.” The FASEB Journal 10, 1025-1030). Though this approach has been relatively successful in clinical trials and leads to rapid replacement of leukemic hematopoiesis with normal polyclonal cells, a fraction of the patients fail to achieve complete remission due to either (i) a collection of symptoms (including hypotension, respiratory distress, and renal failure) referred to as the “ATRA syndrome” or (ii) the development of ATRA resistance by the blast cells. Acquired ATRA resistance is associated with an increase in gene transcription of CRABP II (cytosolic retinoic acid binding protein II) and P450 (Adamson, P. C. et al. (1993) “Time course of induction of metabolism of all-trans retinoic acid and the up-regulation of cellular retinoic acid binding protein.” Cancer Res 53, 472-476). The increased expression of these two proteins leads to higher levels of proteins involved in ATRA catabolism and thus, is proposed to be the reason for low plasma ATRA levels observed in resistant patients undergoing differential therapy. Because this hypercatabolic state persists for several months following the withdrawal of ATRA (and thus places patients at considerable risk) it would be beneficial to APL patients to identify compounds that mimic the effects of ATRA (ATRA “mimetics”) yet are unrecognized by the cell's catabolic machinery.

Cancer is currently the leading cause of death in the United States, being responsible for no fewer than 550,000 deaths each year. An assessment of current cancer treatment regimes finds them lacking as therapeutic reagents. Many of the current chemotherapeutic approaches to cancer treatment (e.g. cisplatinin) fail to exhibit specificity and thus are toxic to a wide range of cell types that divide rapidly. In contrast, ATRA and related compounds appear to be effective agents in the treatment of several cancers; acting to heighten sensitivity of these cells to secondary pharmacological agents and, in the case of APL, directing an irreversible and terminal differentiation of promyelocytic leukemia cells. Thus the RA pathway appears to be one of a select group of recently identified pathways to which new drugs that exhibit a high degree of specificity can be designed. Despite the need for new cancer therapeutics and for a greater understanding of the RA pathway, the art to date has not provided an efficient method of exploring the RA pathway and of identifying new ATRA mimetics and other putative therapeutics. The present invention meets this need.

### BRIEF SUMMARY OF THE INVENTION

The present invention relates to activity of RA-related pathways, as well as to compositions therefrom. More specifically, the present invention generally relates to methods for assessing RA pathway-related activity, and from such methods, obtaining perturbagens with RA-related activity. Such perturbagens then are used to obtain RA-related targets, which in turn can be used to identify potential therapeutics. The invention also provides genetic material for the development of gene therapy agents, vectors and host cells.

The present invention provides polypeptides of Perturbagens R3, 802 and 820, biologically active fragments, analogs and modifications thereof, and polypeptides consisting essentially of such perturbagen sequences. In other aspects, the invention provides polypeptides having at least 99%, at least 95%, at least 90%, at least 85% or at least 80% sequence identitity or homology with such perturbagens, and in other aspects provides N- and C-terminal fragments of such perturbagens. The invention further provides a composition of such polypeptides in a pharmaceutically acceptable carrier, and for treating a RA-related condition with a therapeutically effective amount of a polypeptide of the invention.

The present invention also provides polypeptides having RA pathway activity that are fused to heterologous sequences, in some aspects a scaffold or more particularly, a fluorescent protein scaffold, and provides polypeptides having RA pathway activity that are chemically modified, or more particularly, radiolabelled, acetylated, glycosylated, or fluorescently tagged. Antibodies to the polypeptides of the invention also are provided.

The present invention further provides polynucleotides encoding Perturbagens R3, 802 and 820, biologically active fragments, analogs and modifications thereof, and polypeptides consisting essentially of such perturbagen sequences. In other aspects, the invention provides polynucleotides encoding polypeptides having at least 99%, at least 95%, at least 90%, at least 85% or at least 80% sequence identity or homology with such perturbagens, and in other aspects provides polynucleotides encoding N- and C-terminal fragments of such perturbagens. In some aspects, the polynucleotides are chemically synthesized.

The present invention further provides host cells, vectors, and gene therapy vectors comprising the polynucleotides of the invention. The host cells of the invention further provide for methods for producing RA-related polypeptides by culturing such host cells and recovering such polypeptides.

The present invention also provides methods for identifying a cellular target that interacts with the polypeptides of the invention. In some aspects, the method is performed in vitro and comprises detecting reporter expression, and in particular aspects, utilizes a yeast two-hybrid assay format. The present invention further provides for the use of such target in screening for putative RA-related therapeutics, and in some aspects screens for disruption of polypeptide-target pairs. In particular aspects, a combinatorial chemical library is so screened.

In another aspect, the present invention provides the PAT1 polypeptide, analogs and modifications thereof, and polypeptides consisting essentially of such perturbagen sequences. In other aspects, the invention provides polypeptides having at least 99%, at least 95%, at least 90%, at least 85% or at least 80% sequence identitity or homology with the PAT1 polypeptide.

The present invention further provides polynucleotides encoding the PAT1 polynucleotide, analogs and modifications thereof, and polypeptides consisting essentially of such polypeptide sequences. The present invention further provides host cells, vectors, and gene therapy vectors comprising the polynucleotides of the invention.

### BRIEF DESCRIPTION OF THE DRAWINGS

Figure Legends

FIG. 1. Diagram of PML-RARα chimeric protein. Both the DNA and ligand binding domains of the RARα gene (dark boxes) are retained in the chimeric protein. In addition, the fusion also includes the presumptive DNA-binding zinc-finger domains and leucine zipper of PML.

FIG. 2. Activation of RA-responsive genes. ATRA (small black circles) enters the cell by simple diffusion and binds to RAR homodimers or RAR-RXR heterodimers. Such complexes can translocate across the nuclear membrane and bind to promoter elements referred to as retinoic acid response elements (RARE). Binding of the receptor complex to a RARE promoter activates transcription of the downstream ATRA-responsive gene.

FIG. 3. Diagram of Trans-FACS phenotypic assay. When each cell carries a reporter construct that is sensitive to compound “X” and a member of a perturbagen library, it is possible to isolate perturbagens that activate or suppress expression of the reporter. To identify perturbagens that inhibit or “turn off” the pathway, cells are grown in the presence of an agent (“X”) that normally turns on the pathway. Under these conditions, the majority of cells are bright (unfilled) and cells that carry a perturbagen that inhibits expression of the reporter (shaded cells) can be enriched for and isolated by collecting the “dim” population of cells. Alternatively, to isolate perturbagens that activate a pathway, cells are grown in the absence of “X”. Under these conditions, the majority of the cells are dim (shaded) and perturbagens that induce transcription of the reporter can be isolated by selecting for “bright” cells on the FACS machine.

FIG. 4. A. Mapping the Biologically important region of a perturbagen. Four perturbagens are derived from different breakpoints within the same gene. By mapping the smallest sequence that is common to all four perturbagens (dotted line) it is possible to identify the biologically critical region (black box). B. Critical regions of a gene can be determined by deletion analysis. For instance, a series of N-terminal deletions (dotted line) can be tested for biological activity. In this way, regions of biological importance can be identified.

FIG. 5. Secondary assays. ATRA stimulates the promylocytic cell line, HL60, to differentiate into a granulocyte-like cell. In secondary assays designed to test the breadth of a perturbagen's action, a perturbagen is introduced into HL60 cells and tested for its ability to mimic ATRA action.

FIG. 6. Basic two-hybrid methodology. When bait and prey molecules interact, the Gal4-AD and Gal4-BD binding domains of the Gal4 transcriptional activator are reconstituted. As a result, this functional unit can sit down upon the Gal1 UAS and induce transcription of the reporter gene (lacZ).

FIG. 7. Four-Hybrid System. Host cell RNA targets are identified through a four-hybrid modification of the original two-hybrid scheme. Expanded region (lower left) pictures interaction between “bait” and “target” RNA molecules.

FIG. 8. LANCE™. In the homogeneous assay, a Cy5 labeled perturbagen binds to an Eu-Target molecule in solution. A. When the two molecules are in close proximity, the emissions of the lanthanide chelate can excite Cy5 and give rise to a robust signal. B. In the presence of a small molecule inhibitor, the Cy5-perturbagen-Target-Eu interaction is prevented. Subsequent excitation of Eu results in little or no signal.

FIG. 9. DELFIA™. In the heterogeneous assay, the target is immobilized to a solid support using an Eu labeled monoclonal antibody. Following incubation with the Cy5 labeled perturbagen, the well is washed to remove unbound Cy5. Due to the close proximity of the Eu and Cy5 moieties in the bound complex, excitation of the lanthanide chelate leads to excitation (and emission) of Cy5. In the presence of a small molecule inhibitor (black circles), the Eu-target and Cy5-perturbagen moieties never come in close proximity. In subsequent washes, the free, unbound, Cy5-peptide conjugate is removed and the Eu induced Cy5 signal is insignificant.

FIG. 10. Analysis of pRARβ-EGFP library evolution and individual clones: A. A comparison of autofluorescence of WM35 with the original pRARβ-EGFP library (−ATRA), pRARβ-EGFP library (+ATRA), and the F3 sublibrary (+ATRA). B/C. A comparison of six clones (C1, C5, C7, C8, C9 and C10) in the presence and absence of ATRA.

FIG. 11. Analysis of RARαΔ403. Histogram shows a comparison between the Clone 8 (−ATRA), Clone 8 (+ATRA), and Clone 8, RARαΔ403 (+ATRA).

FIG. 12. Bar Graph showing the four perturbagens that disrupt the RA pathway. Results from both the original (−1) and a second (−2) reading frame are plotted.

FIG. 13a, b. DNA and peptide sequence of four perturbagen clones that inactivate the RARE-GFP reporter.

FIG. 14. Isolation of Activating Perturbagens. A) Bar graph showing the progression of bright clones over the course of six enrichment sorts. B) A histogram comparing Clone 8 cells infected with pVT352 (control) vector with cycle 6 library clones. C) Bar graph showing four activating perturbagens isolated. Note, “OF”=out of frame.

FIG. 15a, b. DNA and peptide sequence of perturbagens that up-regulate the expression of the RARE-GFP reporter construct.

FIG. 16a-d. DNA and peptide sequence of target molecules identified by two-hybrid procedures.

FIG. 17. Plasmid linkage studies showing scaffold dependence of R3-target interactions.

FIG. 18. Western Blot Results. A. Yeast two-hybrid results showing scaffold independence of R3-PAT1 interaction, B. Silver stained gel of HEK293 supernatants immunoprecipitated with anti-GFP antibodies: Lane 1/3: cells co-transfected with dGFP-R3+PAT1, Lane 2/4: cells co-transfected with dGFP-R3(out of frame)+PAT1, C. Western Blot of IP gel stained with anti-PAT1 antibodies, D. Western blot of total protein stained with anti-PAT1 to show all cells expressing PAT1 protein.

FIG. 19a, b. Results of expression profiling studies performed in C8 cells containing the R3 perturbagen in-frame and out of frame.

FIGS. 20-22. Vector diagrams.

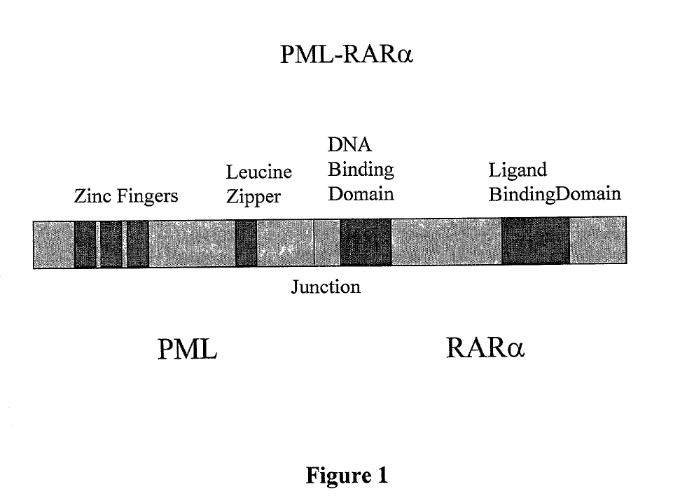

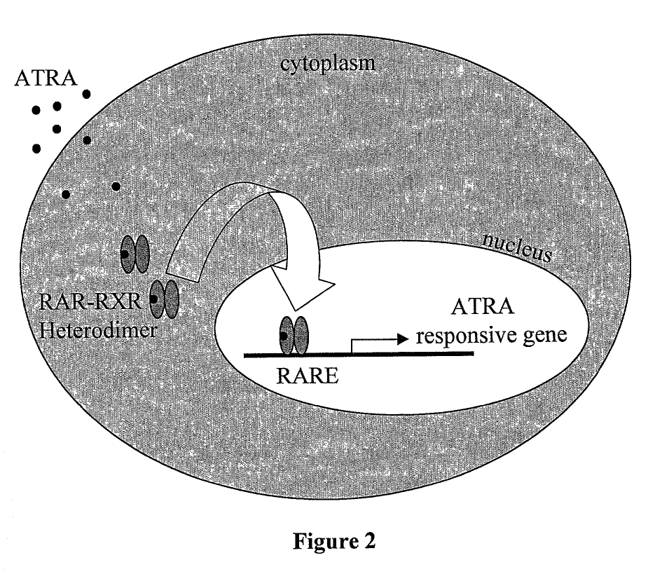

### DEFINITIONS

The terms “perturbagen” or “phenotypic probe” refers to an agent that is proteinaceous or ribonucleic in nature and acts in a transdominant mode to interfere with specific biochemical processes in cells, i.e., through its interaction with specific cellular target(s) or other such component(s), capable of disrupting or activating a particular signaling pathway and/or cellular event. Perturbagens may be encoded by a naturally derived library of compounds such as a cDNA or genomic DNA (gDNA) expression library, or an artificial library comprising synthetic oligonucleotide sequences of a desired length or range of lengths, e.g. a random peptide library. Alternatively, the perturbagen itself can be synthesized using chemical methods. The term “proteinaceous perturbagen” encompasses peptides, oligo- or polypeptides, proteins, protein fragments, or protein variants. Some proteinaceous perturbagens can be as short as three amino acids in length. Alternatively, these agents can be greater than 3 amino acids but less than ten amino acids. Other agents can be greater than ten amino acids but shorter than 30 amino acids in length. Still other agents can be greater than 30 amino acids but less than 100 amino acids in length. Still other agents can be greater than 100 amino acids in length. Naturally occurring proteinaceous perturbagens (i.e. those derived from cDNA or genomic DNA) exhibit a range in size from as little as three to several hundred amino acids. In contrast, synthetic perturbagens (such as those present in a synthetic peptide library) may range in size from three amino acids to fifty amino acids in length and more preferably, from three to 20 amino acids in length, and yet more preferably, about 15 amino acids in length. Similarly, the length of RNA perturbagens can vary. Some RNA perturbagens are as short as 6-10 nucleotides in length. Other RNA perturbagens are between 10 and 50 nucleotides in length. Still other RNA perturbagens are between 50 and 200 nucleotides in length. Other RNA perturbagens are greater than 200 nucleotides in length.

The term “mimetic” refers to a small molecule that (i) exerts the same or similar physiological or phenotypic effect in a bioassay system or in an animal model as does a given perturbagen, or (ii) is capable of displacing a perturbagen from a target in a displacement assay.

The term “small molecule” refers to a chemical compound, for instance a peptide or oligonucleotide that may optionally be derivatized, natural product or any other low molecular weight (less than about 1 kDalton) organic, bioinorganic or inorganic compound, of either natural or synthetic origin. Such small molecules may be a therapeutically deliverable substance or may be further derivatized to facilitate delivery.

The term “target” refers to any cellular component that is directly acted upon by the perturbagen that leads to and/or induces the phenotypic change, detectible for example in a bioassay system.

The terms “library” or “genetic library” refer to a collection of nucleic acid fragments that may individually range in size from about a few to about a million basepairs, with typical expression libraries of about nine to about ten thousand basepairs. These fragments are generated using a variety of techniques familiar to the art.

The term “sublibrary” refers to a portion of a genetic library that has been isolated by application of a specific screening or selection procedure.

The term “insert” in the context of a library refers to an individual DNA fragment that constitutes a single member of the library.

The terms “reporter gene” and “reporter” refer to nucleic acid sequences (or encoded polypeptides) for which screens or selections can be devised. Reporters may be proteins capable of emitting light, or genes that encode intracellular or cell surface proteins detectible by antibodies. Preferably, the reporter activity may be evaluated in a quantitative manner. Alternatively, reporter genes can confer antibiotic resistance.

The term “gene” refers to a DNA substantially encoding an endogenous cellular component, and includes both the coding and antisense strands, the 5′ and 3′ regions that are not transcribed but serve as transcriptional control domains, and transcribed but not expressed domains such as introns (including splice junctions), polyadenylation signals, ribosomal recognition domains, and the like.

The terms “polynucleotide” or “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules. “Oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

The term “fragment” refers to any portion of a proteinaceous perturbagen that is at least 3 amino acids in length, or any RNA molecule that is at least 5 nucleotides in length. The descriptors “biologically relevant” or “biologically active” refer to that portion of a protein or protein fragment, RNA or RNA fragment, or DNA fragment that encodes either of the two previous entities, that is responsible for an observable phenotype (or for activation of a correlative reporter construct).

The term “variant” refers to biologically active forms of the perturbagen sequence (or the polynucleotide sequence that encodes the perturbagen) that differ from the sequence of the initial perturbagen.

The terms “homology” or “homologous” refers to the percentage of residues in a candidate sequence that are identical with the residues in the reference sequence after aligning the two sequences and introducing gaps, if necessary, to achieve the maximum percent of overlap (see, for example, Altschul, S. F. et al. (1990) “Basic local alignment search tool.” J Mol Biol 215(3):403-10; Altschul, S. F. et al. (1997) “Gapped BLAST and PSI-BLAST: a new generation of protein database search programs.” Nucleic Acids Res 25(17):3389-402). It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

The term “scaffold” refers to a proteinaceous or RNA sequence to which the perturbagen is covalently linked to provide e.g., conformational stability and/or protection from degradation.

### DETAILED DESCRIPTION OF THE PREFERRED EMBODIMENTS

Agents isolated from the methods described herein have broad potential and application. Among others, the invention permits the definition of disease pathways, the identification of diagnostically or therapeutically useful targets, and the identification of therapeutic agents. For example, retinoic acid pathway-related genes that are mutated or down-regulated under disease conditions may be involved in causing or exacerbating the disease condition. Treatments directed at up-regulating the activity of such genes or treatments that involve alternate pathways may ameliorate the disease condition. Also, the agents and assays described herein thus have utility as models for diseases related to the retinoic acid pathway. The assays may be utilized as part of a screening strategy designed to identify agents such as compounds that are capable of ameliorating disease symptoms.

As more fully disclosed herein, the described methodology yields first a set of RNA-based or proteinaceous agents, and second, a set of endogenous cellular targets. Each RNA-based or proteinaceous agent (or a mimetic, agonist or antagonist thereof identified through, e.g., routine small molecule screens) may be useful as a direct therapeutic agent in the treatment of cancer and/or various skin diseases. With each such new agent, a corresponding target molecule can be readily identified using standard interaction methodologies such as the two-hybrid technique. Such targets are useful in the development of novel drugs for new chemotherapeutic strategies and may provide useful diagnostic tools for profiling the genetic background (genotype) of the particular disease under study.

A. Overview of the Invention

The invention describes the isolation of new and previously unidentified perturbagens that alter the ability of a cell to detect and respond to all-trans retinoic acid (“ATRA”), and related targets.

The perturbagens described herein were isolated using a phenotypic assay. See priority document U.S. Pat. No. 5,955,275, “Methods for identifying nucleic acid sequences encoding agents that affect cellular phenotypes,” the disclosure of which is incorporated by reference herein in its entirety. Briefly, the assay identifies agents that alter the responsiveness of a cell to ATRA and/or related compounds, including but not limited to 9-cis retinoic acid. To accomplish this, a library polynucleotide sequences is generated using a variety of techniques familiar to the art. After ligating this material into a standard expression vector, the library is transferred into a population of cells of a given type (e.g. a cell line) and screened for sequences that induce a particular biological phenotype. The assay advantageously identifies one or more relevant sequences from the library in the selected host cell population. Cells expressing a biologically relevant perturbagen induce a particular phenotype (or correlative activation of a reporter gene), and are then separated from the rest of the population using, e.g., high-throughput Fluorescent Activated Cell Sorting (FACS) screening procedures. Such high-throughput FACS machines are both highly sensitive and efficient (obtaining screening speeds of approximately 10,000 to up to approximately 65,000 cells or more per minute), thus facilitating identification of biologically relevant sequences that exist at low frequencies within a cell population.

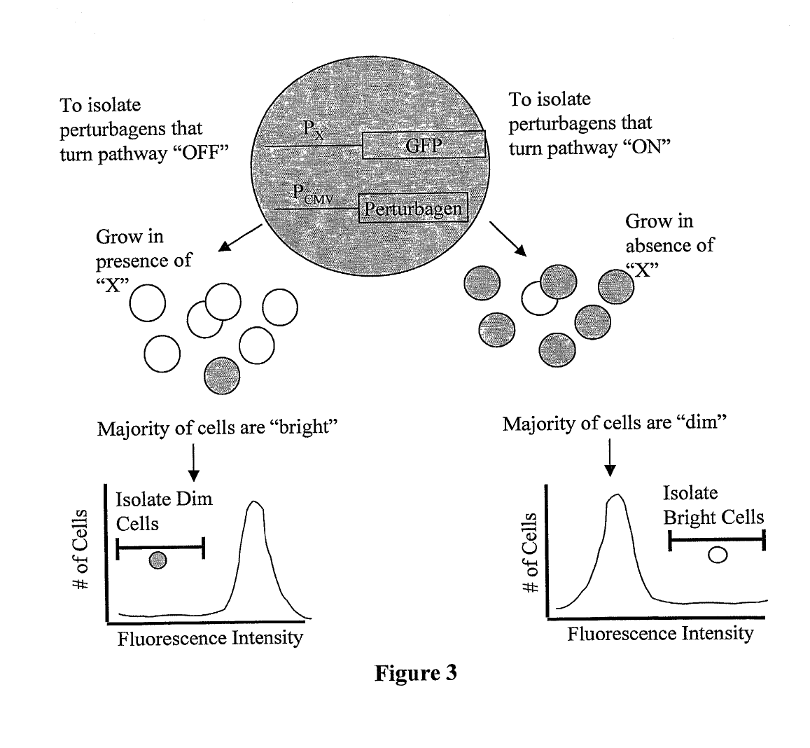

Here, in order to identify molecules that alter the ability of a cell to detect or respond to ATRA, a random primed library of 12×10 clones was constructed from cDNA isolated from placental tissue. This genetic library was transfected into an ATRA-sensitive melanoma cell line that contained a transcriptionally regulated RA-responsive reporter, grown under conditions where RA was limiting and the reporter was initially substantially inactivated. Subsequently, roughly 50 million cells, representing a 4× fold coverage of the library, were subjected to FACS analysis to identify perturbagens that directly or indirectly activated the RA signal (FIG. 3).

Similarly, the methodology is applied to identify molecules that suppress reporter expression. To accomplish this, ATRA-sensitive cells containing the above described reporter and library constructs are grown under conditions where sufficient but non-saturating amounts of ATRA are provided in the medium. Under these conditions, the reporter is activated in the vast majority of cells unless the cell contains a library sequence that directly or indirectly inactivates the RA pathway.

Perturbagen identification may elucidate the function of known genes, or alternatively may work in a black-box approach to identify new genes, gene products, or cellular targets. Thus in some instances, perturbagens may be encoded by a previously identified gene (or gene fragment thereof). Such a gene may be one whose contribution to the disease pathway has previously been identified. Alternatively, the contribution of a gene to the pathway may have been previously unrecognized. In yet other cases, the perturbagen may be found to have no homology with any previously identified polynucleotide or proteinaceous agent. Such perturbagens may be derived from previously unidentified genes, or alternatively may be random sequences that have the proper conformation and/or chemical characteristics needed to alter or modulate one or more components of a pathway(s) that influences the phenotype under investigation. In the methodology described herein, no prior knowledge of the perturbagen or of its corresponding gene, gene product or cellular target is necessary. Moreover, because it is possible for multiple perturbagens to assume similar two or three-dimensional conformations and/or have shared or related chemistries, two or more variants of the same perturbagen may be identified and isolated from a single library without any additional screening steps. Thus one need not spend laborious hours designing, redesigning, or manipulating any candidate molecules, and thus does not bias the experiment with preconceived conceptions of what will or will not induce the phenotype of interest.

B. Phenotypic Probes

The invention encompasses both the phenotypic probes (perturbagens) described herewith and the polynucleotide sequences encoding them. As one of ordinary skill appreciates, such agents may be described by their RNA sequence, amino acid sequence, or correlative DNA sequence. Alternatively, the agents can be sufficiently described in terms of their identity as isolates of a library that exhibit a particular biological activity.

Perturbagens may be encoded by a variety of genetic libraries, including those developed from cDNA, gDNA, and random, synthetic oligonucleotides synthesized using current available methods in chemistry (see, for example, Caponigro et al. (1998) “Transdominant genetic analysis of a growth control pathway.” PNAS 95:7508-7513; Caruthers, M. H. et al. (1980) Nucleic Acids Symposium, Ser. 7:215-223; Horn, T. et al. (1980) Nucleic Acids Symposium, Ser. 7:225-232; Cwirla, S. E. et al. (1990) “Peptides on phage: a vast library of peptides for identifying ligands.” Proc Natl Acad Sci 87(16):6378-82). Alternatively, the perturbagen itself can be synthesized using chemical methods. For example, peptide and RNA synthesis can be performed using various techniques (Roberge, J. Y. et al. (1995) “A strategy for a convergent synthesis of N-linked glycopeptides on a solid support.” Science 269:202-204; Zhang, X. et al. (1997) “RNA synthesis using a universal base-stable allyl linker.” NAR 25(20):3980-3983) and diverse combinatorial peptide libraries can be constructed using, a variety of strategies such as the multipin strategy, the tea bag method, or the split-couple-mix method (see, for instance, Geysen, H. M. et al (1984) “Use of peptide synthesis to probe viral antigens for epitopes to a resolution of a single amino acids.” PNAS 81:3998-4002; Houghten, R. A. (1985) “General methods for the rapid solid phase synthesis of large numbers of peptides: specificity of antigen-antibody interaction at the level of individual amino acids.” PNAS 82:5131-5135; Lam, K. S. et al. (1991) “A new type of synthetic library for identifying ligand binding activity.” Nature 354:82-84; Al-Obeidi, F. et al. (1998) “Peptide and Peptidomimetic Libraries.” Molecular Biotechnology: 9:205-223). Automated synthesis may be achieved using commercially available equipment such as the ABI 431A peptide synthesizer (Perkin-Elmer).

In some cases, the polynucleotide sequence encoding a perturbagen represents a fragment of an existing gene. In such cases, the perturbagen can be readily used to reverse engineer and identify the gene from which the phenotypic probe is derived.

In the case where a perturbagen is encoded by only a portion of a particular gene, the nucleic acid sequence of such a perturbagen may be extended utilizing a partial nucleotide sequence and employing various PCR-based methods known in the art to detect upstream sequences. One such method, restriction site PCR, uses universal and nested primers to amplify unknown sequence from genomic DNA within a cloning vector (Sarkar, G. (1993) “Restriction-site PCR: a direct method of unknown sequence retrieval adjacent to a known locus by using universal primers.” PCR Methods Applic. 2:318-322). Another method, inverse PCR, uses primers that extend in divergent directions to amplify unknown sequence from a circularized template. The template is derived from restriction fragments comprising a known genomic locus and surrounding sequences (see Triglia, T. et al. (1988) “A procedure for in vitro amplification of DNA segments that lie outside the boundaries of known sequences.” NAR. 16:8186). A third method, capture PCR, involves PCR amplification of DNA fragments adjacent to known sequences in human and yeast artificial chromosome DNA (Lagerstrom, M. et al. (1991) “Capture PCR: efficient amplification of DNA fragments adjacent to a known sequence in human and YAC DNA.” PCR Methods Applic. 1:111-119). In this method, multiple restriction enzyme digestions and ligations may be used to insert an engineered double stranded sequence into a region of known sequence before performing PCR. Other methods which may be used to retrieve unknown sequences are known in the art (Parker, J. D. et al (1991) “Targeted gene walking polymerase chain reaction.” NAR. 19:3055-3060). In addition, one may use nested primers and PROMOTERFINDER libraries (Clontech, Palo Alto, Calif.) to walk genomic DNA. This procedure avoids the need to screen libraries and is useful in finding intron/exon junctions. For all PCR based methods, primers may be designed, using commercially available software such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

In one particular embodiment, the invention encompasses proteinaceous perturbagens, biologically active fragments, (N-terminal, C-terminal, or central) or variants thereof. Proteinaceous perturbagens can exert their effects by multiple means. For example, a peptide may act by binding and disrupting the interactions between two or more proteinaceous entities within the cell. Alternatively, a peptide perturbagen can bind to, and disrupt translation of a particular mRNA molecule. As still another alternative, peptide perturbagens may bind to genomic DNA and disrupt gene expression by altering the ability of one or more transcription factor(s) (e.g. activators or repressors) from binding to a critical enhancer/promoter region of the regulatory region of the gene.

Penetrance is another property of perturbagens. Penetrance is defined as the number of cells exhibiting a particular phenotype divided by the total number of cells in the experiment (when a perturbagen is present in the cells), minus the total number of cells exhibiting a particular phenotype divided by the total number of cells in the experiment when the perturbagen is not present in the cells. The penetrance of any given pertubagen can vary depending upon a variety of parameters including 1) the cell type it is being expressed in, 2) the vector being used to express the perturbagen, 3) the biological stability (half-life) of the perturbagen or mRNA encoding the perturbagen 4) the concentration of the perturbagen in the cell, as well as other parameters. Thus although penetrance is a factor that impacts how immediately a given perturbagen can be seen to exert an effect, in some instances a desirable, biologically active perturbagen may present a relatively low rate of penetrance. As one of ordinary skill will appreciate, perturbagens of low penetrance may be obtained and manipulated via standard cycling and/or amplification procedures. Thus, some preferred perturbagens may exhibit as low as 1-2% penetrance. Other preferred perturbagens may exhibit between 2% and 5% penetrance, between 5 and 10% penetrance, 10% and 20% penetrance, between 20% and 50% penetrance, or even in some instances, between 50% and 100% penetrance.

In some instances, the action, penetrance, or biological activity of a perturbagen may be affected in some part by the scaffold to which it is associated. In some cases (for instance, in situations where the agent is shorter than 30 amino acids) the scaffold may drive the perturbagen to adopt a conformation that enhances its biological action. In still other instances, one or more neighboring residues from, e.g., the C-terminus of a scaffold, may act in concert with the perturbagen to enhance the functionality of the molecule. In cases such as these, the complete biologically active sequence may include one or more C-terminal residues derived from the scaffold molecule. Multiple techniques may be used to determine the contribution of the scaffold to the phenotypic effect of any given perturbagen. Initially, perturbagen sequences can be shifted to alternative scaffolds and retested for biological activity. If these procedures result in a significant loss of the perturbagen's activity, a fusion between the perturbagen and, for instance, the 30-most residues from the C-terminus of the scaffold may be linked to a second scaffold molecule and retested for biological activity. Should operations such as these lead to the recovery of lost activity, experiments in which smaller and small portions of the scaffold are associated with the perturbagen can be tested.

In other embodiments, the phenotypic probe is an RNA molecule which is itself active (i.e. is not acting through the correlative encoded protein or peptide that results from translation of the RNA). There are multiple mechanisms by which RNA molecules may act to inhibit or activate a biological pathway. In some instances, the RNA perturbagen acts in an antisense mode to disrupt ribonucleic acid transcription or translation of a cellular mRNA target via hybridization to a target ribonucleic acid (Weiss, B. et al.(1999) “Antisense RNA gene therapy for studying and modulating biological processes.” Cell Mol Life Sci. 55(3):334-58). In this context the term “antisense” refers to any composition containing a nucleic acid sequence which is complementary to the “sense” strand of a particular target DNA. In other instances, RNA perturbagens may act as a RNA-PRO agents, disrupting or activating the ATRA pathway by interacting with proteinaceous components of the cell (see Sengupta, D. J. (1999) “Identification of RNAs that bind to a specific protein using the yeast three-hybrid system.” RNA 5:596-601). In still other instances, RNA agents act as a triplex-forming oligonucleotide (TFO) agents to interact with promoter sequences, exons, introns, or other portions of genomic DNA to disrupt or activate transcription of components of the ATRA pathway (see Postel, E. H. et al. (1989) “Evidence that a triplex-forming oligonucleotide binds to the c-myc promoter in HeLa cells, thereby reducing c-myc RNA levels.” PNAS 88:8227-8231; Svinarchuk, F. et al. (1997) “Recruitment of transcription factors to the target site by triplex-forming oligonucleotides.” NAR 25:3459-3464.

There does not appear to be a necessary correlation between size of a particular RNA (or proteinaceous) perturbagen and penetrance. Instead, penetrance of RNA perturbagens are dependent upon the perturbagen stability or half-life, the perturbagen's ability to achieve access to the target molecule, and other factors.

Perturbagens may also exhibit cross-reactivity. A variety of host target proteins can contain similarities in both the primary and secondary structure. As a result, one or more of the agents described herein may exhibit affinity for one or more target variants/isoforms present in nature. Similarly, agents identified in the following screens may exhibit affinity for two or more functionally unrelated proteins that contain regions or domains that share homology or related functional groups. Thus, for instance, a perturbagen that recognizes a zinc-binding domain of one protein may also show affinity for the homologous (and functionally equivalent) region of a second protein (see, e.g., Mavromatis K. O. et al. (1997) “The carboxyl-terminal zinc-binding domain of the human papillomavirus E7 protein can be functionally replaced by the homologous sequences of the E6 protein.” Viral Research 52(1):109-18). In cases where such interactions lead to relevant biological phenotypes, the underlying mechanism(s) may differ considerably from those brought about by the original perturbagen-target interactions. Furthermore, in cases where an agent exhibits cross reactivity with secondary targets, said agents may be useful in a broader set of therapeutic and diagnostic applications than originally intended.

Host range is another characteristic of perturbagens. The term “host range” refers to the breadth of potential host cells that exhibit perturbagen-induced phenotypes. In some instances, such as the case where the perturbagen is represented by an apoptosis-inducing fragment of BID, the host range is broad, due to the near ubiquitous participation of BID or BID-like agents in the apoptotic pathway of many cells. In contrast, some perturbagens have a very limited host range due to, for instance, the restricted expression of the perturbagen target.

C. Sequence Variants

In another embodiment, the invention includes sequence variants of both the phenotypic probes and the polynucleotide sequences that encode them. Thus, in the case of proteinaceous perturbagens, variants contain at least one amino acid substitution, deletion, or insertion from the original isolated form of the perturbagen that provides biological properties that are substantially similar to those of the initial perturbagen. Similarly, variants of RNA-based phenotypic probes contain at least one nucleotide substitution, deletion, or insertion when compared to the original isolated sequence.

In addition to being described by their respective sequence, variants may also be identified by the relative amounts of homology they have in common with the original perturbagen sequence. Alternatively, a variant of a proteinaceous perturbagen may be described in terms of defining the nature of an amino acid substitution. “Conservative” substitutions are those in which the substituting residue is structurally or functionally similar to the substituted residue. In non-conservative substitutions, the substituting and substituted residue will be from structurally or functionally different classes. For the purposes herein, these classes are as follows: 1. Electropositive: R, K, H; 2. Electronegative: D,E; 3. Aliphatic: V,L,I,M; 4. Aromatic: F,Y,W; 5. Small: A,S,T,G,P,C; 6. Charged: R,K,D,E,H; 7. Polar: S,T,Q,N,Y,H,W; and Small Hydrophilic: C,S,T. Interclass substitutions generally are characterized as nonconservative, while intraclass substitutions are considered to be conservative. In some instances, variant polypeptides sequences can have 65-75% homology with the original agent. In other embodiments, variants have between 75% and 85% homology with the original agent. In still other embodiments, variants will have between 85% and 95% homology with the original perturbagen agent. In yet other embodiments, variants have between 95% and greater than 99% polypeptide sequence identity with the original perturbagen agent. In some cases, the homology between two perturbagens (variants) is confined to a small region of the molecule (e.g. a motif). Such conserved sequences are often indicative of regions that contain biologically important functions and suggest the perturbagens share a common cellular target. In these situations, while only limited and conservative amino acid changes are desirable within the region of the motif, greater levels of variation can exist in adjacent and more distal portions of the polypeptide.

Like their proteinaceous counterparts, variants of RNA perturbagens may also be described in terms of percent homology. In some instances, the variant ribonucleotide sequences can have 65-75% homology with the original agent. In other embodiments, the variants have between 75% and 85% homology with the original agent or between 85% and 95% homology with the original perturbagen sequence, or even between 95% and greater than 99% sequence identity with the original perturbagen agent. Again, greater variation can, in some embodiments, exist outside an identified region/motif without altering biological activity.

Lastly, in reference to the DNA sequences encoding proteinaceous perturbagens, one who is skilled in the art will appreciate that the degree of variance will depend upon and/or reflect the degeneracy of the genetic code. As one in the art appreciates, a given phenotypic probe is encoded by a selection of polynucleotide sequences. Therefore, the invention encompasses each variation of polynucleotide sequence that encodes the given perturbagen, such variations being made in accordance with the standard triplet genetic code as applied to the polynucleotide sequence of each perturbagen. For each proteinaceous perturbagen described by amino acid sequence herein, all such corresponding DNA variations are to be considered as being specifically disclosed.

Variants of phenotypic probes may arise by a variety of means. Some variants may be artifactual and result from, for instance, errors that occur in the process of PCR amplification or cloning of the perturbagen encoding sequence. Alternatively, variants may be constructed intentionally. For instance, it may be advantageous to produce nucleotide sequences encoding perturbagens possessing a substantially different codon usage. Codons may be selected to increase the rate at which expression of the peptide or RNA occurs in a particular prokaryotic or eukaryotic cell in accordance with the frequency with which particular codons are utilized by the host (Berg, O. G. (1997) “Growth rate-optimized tRNA abundance and codon usage.” J Mol Biol 270(4):544-50). Additional reasons for substantially altering the nucleotide sequence encoding proteinaceous perturbagens (without altering the encoded amino acid sequences) include, but are not limited to, producing RNA transcripts that have increased half-life. This may be accomplished by altering a sequence's structural stability (see, for example, Gross, G. et al. (1990) “RNA primary sequence or secondary structure in the translational initiation region controls expression of two variant interferon-beta genes in Escherichia coli.” J Biol Chem. 265(29):17627-36; Ralston, C. Y. et al. (2000) “Stability and cooperativity of individual tertiary contacts in RNA revealed through chemical denaturation.” Nat Struct Biol. 7(5):371-4), or through addition of untranslated sequences that increase RNA stability/half-life through RNA-protein interactions (see, for example, Wang ,W. et al. (2000) “HuR regulates cyclin A and cyclin B1 mRNA stability during cell proliferation.” EMBO J. 19(10):2340-50; Shetty, S. and Idell, S. (2000) “Posttranscriptional regulation of plasminogen activator inhibitor-1 in human lung carcinoma cells in vitro.” Am J Physiol Lung Cell Mol Physiol 278(1):L148-56). Also included the category of intentional variants are those whose sequence has been altered in order to add or deleted sites involved in post-translational modification. Included in this list are variants in which phosphorylation sites, acetylation sites, methylation sites, and/or glycosylation sites have been added or deleted (see, for example, Wicker-Planquart, C. (1999) “Site-directed removal of N-glycosylation sites in human gastric lipase.” Eur J Biochem. 262(3):644-51; Dou, Y. (1999) “Phos-phorylation of linker histone H1 regulates gene expression in vivo by mimicking H1 removal.” Mol Cell. 4(4):641-7).

Variants may also arise as a result of simple and relatively routine techniques involving random mutagenesis or DNA shuffling; procedures that are often used to rapidly evolve perturbagen encoding sequences and allow identification of variants that have increased biological stability or activity (see, for instance, Ner, S. S. et al. (1988) “A simple and efficient procedure for generating random point mutations and for codon replacements using mixed oligonucleotides.” DNA 7:127-134; Stemmer, W. (1994) “Rapid evolution of a protein in vitro by DNA shuffling.” Nature 370:389-391). For instance, in mutagenic PCR, the fragment encoding the perturbagen is PCR amplified under conditions that increase the error rate of Taq polymerase. This is accomplished by i) increasing the MgCl₂ concentrations to stabilize non-complementary pairings, ii) addition of MnCl₂ to diminish template specificity of the polymerase and iii) increasing the concentration of dCTP and dTTP to promote misincorporation of basepairs in the reaction. As a result of this process, the error rate of Taq polymerase may be increased from 1.0×10⁻⁴ errors per nucleotide per pass of the polymerase, to approximately 7×10⁻³ errors per nucleotide per pass. Amplifying a perturbagen-encoding sequence under these conditions allows the development of a library of dissimilar sequences which can subsequently be screened for variants that exhibit improved biological activity.

In addition to variants that are created by artificial or accidental means, natural variants may also exist. For instance, in the course of screening any given genomic or cDNA library, it is possible that a perturbagen, derived from a sequence that exists in multiple copies within the genome (e.g. duplications, repetitive sequences), may be isolated numerous times. Such sequences often contain polymorphisms that result in alterations in the encoded RNA and polypeptide sequence (see, for example, Satoh, H. et al. (1999) “Molecular cloning and characterization of two sets of alpha-theta genes in the rat alpha-like globin gene cluster.” Gene 230(1):91-9) and thus, may represent natural variants of the perturbagen agent. Alternatively, if multiple libraries are utilized to screen for perturbagens and two or more of those libraries are derived from unrelated individuals, dissimilar tissues, or contrary periods in the development of a tissue (e.g., adult vs. fetal tissue), it is possible that variants may be isolated as a result of allelic variation (see, for example, Posnett, D. N. (1990) “Allelic variations of human TCR V gene products.” Immunol Today. 11(10):368-73). Variants of phenotypic probes may arise by these and other means.

Variants of any given perturbagen may in some instances exhibit additional biological properties. For instance, perturbagens that previously recognized only a single target may demonstrate broadened specificity, e.g., may bind multiple isoforms or serotypes of a target in response to the alteration of a single amino acid in the perturbagen variant. Similarly, a perturbagen having a specific phenotype in one cell may exhibit additional phenotypes or may exhibit a broader effective host range after making small alterations in perturbagen variant sequence.

D. Biologically Active Fragments

Some embodiments of the invention encompass biologically active fragments of a given proteinaceous or RNA-based perturbagen. Biologically active fragments may be compromised of N-terminal, C-terminal, or internal fragments of peptide perturbagens, or 5′, 3′ or internal fragments of RNA perturbagens. In some instances, the fragment encodes or represents portions of a natural gene. In other instances the fragment is derived from a larger polynucleotide or polypeptide that has no known natural counterpart. In still other instances, biologically active regions of a perturbagen can be artificially synthesized (by chemical or recombinant methods) so that multiple, tandem copies of the phenotypic probe are covalently linked together and expressed. All such biologically active perturbagen fragments are, in turn, encoded by a variety of correlative DNA sequences.

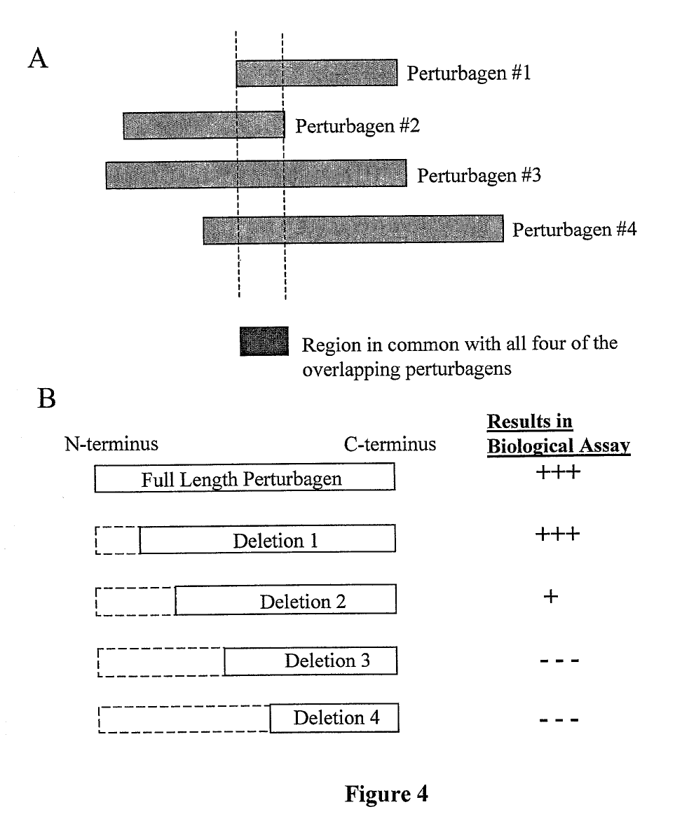

The biologically active portion of a molecule can be identified by several means. In some instances, biological relevant regions can be deduced by simple physical mapping of families of overlapping sequences isolated from a phenotypic assay (Hingorani, K. et al. (2000) “Mapping the functional domains of nucleolar protein B23.” J. Biol Chem May 26). For instance, in the course of any given screen, multiple perturbagens, derived from alternative breakpoints of the same gene, may be isolated from one or more genetic libraries. (FIG. 4). The smallest region that is common to all of the perturbagens can demarcate the area of biological importance.

Alternatively, critical regions of a perturbagen can frequently be distinguished by comparing the polynucleotide and/or amino acid sequence of two or more perturbagens that share a common target (see, for example, Grundy, W. N. (1998) “Homology detection via family pair-wise search.” J Comput Biol. 5(3):479-9; Gorodkin, J. et al. (1997) “Finding common sequence and structure motifs in a set of RNA sequences.” Ismb 5:120-3). In this instance, conserved sequences (or motifs) that are identified by this form of analysis often provide important clues necessary to determine biologically important regions of a given molecule. Alternatively, methods that identify biologically relevant regions by altering or deleting regions of the perturbagen molecule can also be used. For instance, the gene encoding a particular perturbagen can be subjected to deletion analysis whereby portions of the gene are removed in a systematic fashion, thus allowing the remaining entity to be retested for its ability to evoke a biological response (see, for example, Huhn, J. et al. (2000) “Molecular analysis of CD26-mediated signal transduction in cells.” Immunol Lett 72(2):127-132; Davezac, N. et al. (2000) “Regulation of CDC25B phosphatases subcellular localization.” Oncogene 19(18):2179-85).

Alternatively, biologically critical regions of a molecule can be identified by inducing mutations in the sequence encoding the polypeptide (see, for example, Ito, Y. et al. (1999) “Analysis of functional regions of YPM, a superantigen derived from gram-negative bacteria.” Eur J Biochem; 263(2):326-37; Kim, S. W. et al. (2000) “Identification of functionally important amino acid residues within the C2-domain of human factor V using alanine-scanning mutagenesis.” Biochemistry 39(8):1951-8.). Subsequent testing of the variants of said molecule for biological activity enables the investigator to identify regions of the perturbagen that are both critical and sensitive to manipulation. Furthermore, molecular probes such as monoclonal antibodies and epitope-specific peptides can be useful in the identification of biologically important regions of a perturbagen (see, for example, Midgley, C. A. et al. (2000) “An N-terminal p14ARF peptide blocks Mdm2-dependent ubiquitination in vitro and can activate p53 in vivo.” Oncogene 19(19):2312-23; Lu, D. et al. (2000) “Identification of the residues in the extracellular region of KDR important for interaction with vascular endothelial growth factor and neutralizing anti-KDR antibodies.” J Biol Chem 275(19):14321-30). In this procedure, probes that bind and thus mask specific regions of a perturbagen can be tested for their ability to block the biological activity of the molecule. These techniques (as well as others) can be used to map the boundaries of any given biologically active residues.

E. Heterologous Sequences

In another embodiment, the invention encompasses all heterologous forms of the phenotypic probes and the polynucleotide sequences encoding them described herewith. In this context, “heterologous sequence(s)” include versions of the perturbagens that are i) scaffolded by other entities, ii) tagged with marker sequences that can be recognized by antibodies or specific peptides, iii) altered to transform post-translational patterns of modification, or iv) altered chemically so as to cyclicize the molecule for alternative pharmacodynamic/pharmacokinetic properties.

1. Scaffolds

Peptide perturbagens can be fused to protein scaffolds at N-terminal, C-terminal, or internal sites. Similarly, RNA derived perturbagens can be fused to RNA sequences at 5′, 3′ or internal sites. The fusion of a perturbagen to a second entity can increase the relative effectiveness of the perturbagen by increasing the stability of either the messenger RNA (mRNA) or protein of said agent. In some instances, scaffolds may be a relatively inert protein, (i.e. having no enzymatic activity or fluorescent properties) such as hemagglutinin. Such proteins can be stably expressed in a wide variety of cell types without disrupting the normal physiological functions of the cell. In other instances, scaffolds may serve a dual function, e.g., increasing perturbagen stability while at the same time, serving as an indicator or gauge of the level of perturbagen expression. In this case, the scaffold may be an autofluorescent molecule such as a green fluorescent protein (Clontech) or embody an enzymatic activity capable of altering a substrate in such a way that it can be detected by eye or instrumentation (e.g. β galactosidase). For example, in the invention described herein, various molecular techniques that are common to the field are used to link the perturbagen library to, e.g., the C-terminus of a nonfluorescent variant of GFP. “dEGFP” (also referred to as “dead-GFP”) is one such nonfluorescent variant brought about by conversion of Tyr→Phe at codon 66 of EGFP (Clontech). By linking the perturbagen library to this molecule, each library member is fused to a separate dEGFP molecule. Such chimeric fusions can easily be detected by Western Blot analysis using antibodies directed against GFP and are useful in determination of intracellular expression levels of perturbagens. In addition, by modifying the perturbagen sequences or the scaffold to which they are attached with various localization signals, the perturbagen may be directed to a particular compartment within the host cell. For example, proteinaceous perturbagens can be directed to the nucleus of certain cell types by attachment of a nuclear localization sequence (NLS); a heterogeneous sequence made up of short stretches of basic amino acid residues recognized by importins alpha and/or beta.

2. Antibody-Tagged Perturbagens

Perturbagens can be constructed to contain a heterologous moiety (a “tag”) that is recognized by a commercially available antibody. Such heterologous forms may facilitate studies of subjects including, but not limited to, i) perturbagen subcellular localization, ii) intracellular concentration assessment and iii) target binding interactions. In addition, the tagging of a perturbagen may also facilitate purification of fusion proteins using commercially available matrices (see, for example, James, E. A. et al. “Production and characterization of biologically active human GM-CSF secreted by genetically modified plant cells.” Protein Expr Purif. 19(1):131-8; Kilic, F. and Rudnick, G. (2000) “Oligomerization of serotonin transporter and its functional consequences.” Proc Natl Acad Sci U S A. 97(7):3106-11). Such tag moieties include, but are not limited to glutathione-S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc and HA enable immunoaffinity purification of fusion proteins using commercially available monoclonal and polyclonal antibodies that specifically recognize these epitope tags. Such fusion proteins may also be engineered to contain a proteolytic cleavage site located between the perturbagen sequence and the heterologous protein sequence, so that the perturbagen may be cleaved away from the heterologous moiety following purification. A variety of commercially available kits may be used to facilitate expression and purification of fusion proteins.

3. Chemically Modified Perturbagens

In addition to the chimeric variants described above, chemical modification encompass a variety of modifications including, but not limited to, perturbagens that have been radiolabeled with ³²P or ³⁵S, acetylated, glycosylated, or labeled with fluorescent molecules such as FITC or rhodamine. These modifications may be directly imposed on the perturbagen itself (see, for example, Shuvaev, V. V. et al. (1999) “Glycation of apolipoprotein E impairs its binding to heparin: identification of the major glycation site.” Biochim Biophys Acta 1454(3):296-308; Dobransky, T. et al. (2000) “Expression, purification and characterization of recombinant human choline acetyltransferase: phosphorylation of the enzyme regulates catalytic activity.” Biochem J. 349(Pt 1):141-151). Alternatively, changes may be made to the polynucleotide sequence encoding the perturbagen so as to alter the pattern of phosphorylation, acetylation, or glycosylation, or to that lead to cyclization of peptides in order to alter membrane permeability and/or pharmacodynamic-pharmacokinetic properties (see, for example, Borchardt, R. T. (1999) “Optimizing oral adsorption of peptides using prodrug strategies.” J Control Release 62(1-2):231-8.).

F. Hybridization

The invention also encompasses polynucleotide sequences that are capable of hybridizing to the claimed polynucleotide sequences encoding phenotypic probes and said variants of such entities described previously, under various conditions of stringency. Such reagents may be useful in i) therapeutics, ii) diagnostic assays, iii) immunocytology, iv) target identification, and v) purification. For example, if the sequence encoding a particular perturbagen is introduced into a subject for gene therapeutic purposes, it may be necessary to monitor the success of integration and the levels of expression of said agent by Southern and Northern Blot analysis respectively (Pu, P. et al. (2000) “Inhibitory effect of antisense epidermal growth factor receptor RNA on the proliferation of rat C6 glioma cells in vitro and in vivo.” J Neurosurg. 92(1):132-9). In other instances, hybridization may be used as a tool to define or describe a perturbagen variant or fragment, and a hybridizing sequence thus may have direct relevance as an ATRA mimetic or other such therapeutic agent.

The term “hybridization” refers to any process by which a strand of nucleic acid binds with a complementary or near-complementary strand through base pairing. There are several parameters that play a role in determining whether two polynucleotide molecules will hybridize including salt concentrations, temperature, and the presence or absence of organic solvents. For instance stringent salt concentrations will ordinarily be less than about 750 mM NaCl and 75 mM trisodium citrate, preferably less than about 500 mM NaCl and 50 mM trisodiium citrate, and most preferably less than about 250 mM NaCl and 25 mM trisodium citrate. Low stringency hybridization can be obtained in the absence of organic solvent (e.g. formamide) while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent and the inclusion or exclusion of carrier DNA are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide and 100 ug/ml denatured salmon sperm DNA (ssDNA). In a most preferred embodiment, hybridization will occur at 42° C. in 250 mM NaCl, 25 mM trisodium citrate, 1% SDS, 50% formamide and 200 ug/ml denatured ssDNA. Useful variations on these conditions will be readily apparent to those skilled in the art.

The washing steps that follow hybridization can also vary greatly in stringency. Wash stringency conditions can be defined by salt concentration and by temperature. As above, wash stringency can be increased by decreasing salt concentration or by increasing temperature. For example, stringent salt concentrations for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperatures of at least about 25° C., more preferably of at least about 42° C., and most preferably of at least about 68° C. In a preferred embodiment, wash steps will occur at 25° C. in 30 mM NaCl, 3 mM trisodium citrate and 0.1% SDS. In a more preferred embodiment, wash steps will occur at 42° C. in 15 mM NaCl, 1.5 mM trisodium citrate and 0.1% SDS. In a most preferred embodiment, wash steps will occur at 68° C. in 15 mM NaCl, 1.5 mM trisodium citrate and 0.1% SDS. Additional variations on these conditions will be readily apparent to those skilled in the art.

G. Expression Vectors

The DNA sequence encoding each perturbagen or target (or variant or fragment thereof) may be inserted into an expression vector which contains the necessary elements for transcriptional/translational control in a selected host cell. Thus the DNA sequence may be expressed for, e.g., testing in a bioassay such as those described herein, or in a binding assay such as those described herein, or for production and recovery of the proteinaceous agent. Methods which are well known to those skilled in the art are used to construct expression vectors containing sequences encoding the perturbagens and the appropriate transcriptional and translational control elements. These methods include in vitro recombinant DNA techniques, synthetic techniques, and in vivo genetic recombination (see Sambrook, J. et al. (1989) “Molecular Cloning, A Laboratory Manual”, Cold Spring Harbor Press, Plainview N.Y.).

Exemplary expression vectors may include one or more of the following: (i) regulatory sequences, such as enhancers, constitutive and inducible promoters, and/or (ii) 5′ and 3′ untranslated regions, and/or (iii) mRNA stabilizing sequences or scaffolds, for optimal expression of the perturbagen in a given host. For instance, intracellular perturbagen levels can be modulated using alternative promoter sequences such as CMV, RSV, and SV40 promoters, to drive transcription (see, for example, Zarrin, A. A. et al. (1999) “Comparison of CMV, RSV, SV40 viral and Vlambda1 cellular promoters in B and T lymphoid and non-lymphoid cell lines.” Biochim Biophys Acta. 1446(1-2):135-9). Alternatively, inducible promoter systems, (e.g. ponesterone-induced promoter (PIND, Invitrogen, see Dunlop, J. et al. (1999) “Steroid hormone-inducible expression of the GLT-1 subtype of high-affinity 1-glutamate transporter in human embryonic kidney cells.” Biochem Biophys Res Commun. 265(1):101-5), tissue specific enhancers (see Scharf, D. et al. (1994) Results Probl. Cell Differ. 20:125-162), or scaffolding molecules (see, for example, see Abedi, M. et al. (1998), “Green fluorescent protein as a scaffold for intracellular presentation of peptides.” Nucleic Acid Research 26(2):623-630) can be used to modulate intracellular perturbagen levels.

A variety of paired expression vector/host systems may be utilized to contain and express sequences encoding the perturbagens. As one of ordinary skill will appreciate, the selection of a given system is dictated by the purpose of expression: e.g., bioassay, binding assay, or production of proteinaceous product for subsequent isolation and purification. Such systems include, but are not limited to, microorganisms such as bacteria transformed with recombinant bacteriophage, plasmid or cosmid DNA expression vectors; yeast transformed with yeast expression vectors, insect cell systems infected with viral expression vectors (e.g. baculovirus), plant cell systems transformed with viral expression vectors (e.g. tobacco mosaic virus, TMV) or with bacterial expression vectors (e.g. Ti or pBR322 plasmids; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter). The host cell employed does not limit the invention.

In bacterial systems, a number of cloning and expression vectors may be selected depending upon the use intended for polynucleotide sequences encoding the perturbagens. For example, routine cloning, subcloning, and propagation of polynucleotide sequences encoding perturbagens can be achieved using a multifunctional E. coli vector such as PBLUESCRIPT (Stratagene, La Jolla Calif.). Ligation of sequences encoding perturbagens into the vector's cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (see e.g., Van Heeke, G. and Schuster, S. M. (1989) “Expression of human asparagine synthetase in Escherichia coli.” J. Biol. Chem. 264:5503-5509). When large quantities of perturbagens are needed, e.g. for the production of antibodies, vectors which direct high level expression of perturbagens may be used. Exemplary vectors feature the strong, inducible T5 or T7 bacteriophage promoter; the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned anaphylatoxin C3a receptor gene protein can be released from the GST moiety.

Yeast expression systems may also be used for production of perturbagens. A number of vectors containing constitutive or inducible promoters such as alpha factor, alcohol oxidase, and PGH promoters, may be used in the yeast Saccharomyces cerivisiae or related strains. In addition, such vectors can be designed to direct either the secretion or intracellular retention of expressed proteins and enable integration of foreign sequences in the host genome for stable propagation. (see, e.g. Bitter, G. A. et al. (1987) “Expression and secretion vectors for yeast.” Methods Enzymology. 153:516-544; and Scorer, C. A. et al. (1994) “Rapid selection using G418 of high copy number transformants of Pichia pastoris for high-level foreign gene expression.” Biotechnology 12:181-184).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g., see Logan et al., Proc. Natl. Acad. Sci. USA, 81:3655-59 (1984)). Specific initiation signals may be used to achieve more efficient translation of sequences encoding the perturbagen. Such signals include the ATG initiation codon and adjacent sequences, e.g. the Kozak sequence. In cases where sequences encoding the perturbagen and its initiation codon and upstream regulatory sequences are inserted into the appropriate expression vector, no additional transcriptional or translational control signals may be needed. However, in cases where only coding sequence is inserted, exogenous translational control signals including an in-frame ATG initiation codon are provided by the vector. Furthermore, the initiation codon must be in phase with the reading frame of the desired coding sequence to ensure translation of the entire insert. Such exogenous translational elements and initiation codons may be of various origins, both natural and synthetic. The efficiency of expression may be enhanced by the inclusion of appropriate transcription enhancer elements, transcription terminators, etc. (see Bitter, et al., Methods in Enzymol., 153:516-44 (1987)). Alternatively, many of these elements are not required in vectors that are specific for RNA-based perturbagens. Instead, sequences that stabilize the RNA transcript or direct the RNA sequence to a particular compartment will be included (see, for instance, Wood Chuck post transcriptional regulatory element, WPRE, Zufferey, R. et al. (1999) “Woodchuck hepatitis virus posttranscriptional regulatory element enhances expression of transgenes delivered by retroviral vectors.” J Virol 73(4):2886-92).

Plant systems may also be used for expression of perturbagens. Transcription of sequences encoding perturbagens may be driven by viral promoters, e.g. the 35S and 19S promoters of CaMV used alone or in combination with the omega leader sequence from TMV (Takamatsu, N. (1991) “Deletion analysis of the 5′ untranslated leader sequence of tobacco mosaic virus RNA.” J Virology 65:1619-22). Alternatively, plant promoters such as the small subunit of RUBISCO or heat shock promoters may be used. (see, for example, Coruzzi, G. et al. (1984) “Tissue-specific and light-regulated expression of a pea nuclear gene encoding the small subunit of ribulose-1,5-bisphosphate.” EMBO J. 3:1671-80; Broglie, R. et al. (1984) “Light-regulated expression of a pea ribulose-1,5-bisphosphate carboxylase small subunit gene in transformed plant cells.” Science 24:838-843).

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith, et al., J. Virol. 46:584-93 (1983); U.S. Pat. No. 4,745,051).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

The selected construct can be introduced into the selected host cell by direct DNA transformation or pathogen-mediated transfection. The terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Preferred technologies for introducing perturbagens into mammalian cells include, but are not limited to, retroviral infection as well as transformation by EBV or similar episomally-maintained viral vectors (Makrides, S. C. (1999) “Components of vectors for gene transfer and expression in mammalian cells.” Protein Expr Purif 17(2):183-202). Other suitable methods for transforming or transfecting host cells can be found in Maniatis, T. et al (“Molecular Cloning: A Laboratory Manual.” Cold Spring Harbor Laboratory Press) and other standard laboratory manuals.

For long term production of recombinant proteins in mammalian systems, stable expression of perturbagens in cell lines is preferred. For example, sequences encoding perturbagens can be transformed or introduced into cell lines using expression vectors which may contain viral origins of replication and/or endogenous expression elements and a selectable marker gene on the same or on a separate vector. Alternatively, cells can be transfected using, for instance, retroviral, adenoviral, or adeno-associated viral agents as delivery systems for the perturbagen. For example, retroviral vectors (e.g. LRCX, Clontech) may be used to introduce and express perturbagens in a variety of mammalian cell cultures. Such vectors may rely on the virus' own 5′ LTR as a means of driving perturbagen expression or may utilize alternative promoters/enhancers (e.g. those of CMV, RSV and SV40, PIND) to regulate perturbagen expression levels.

In a preferred embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include but are not limited to polyclonal, monoclonal, chimeric, single chain, Fab fragments and fragments produced by a Fab expression library.

In some instances, a preliminary selection is performed to verify that the host cells have been successfully transformed/transfected. Following the introduction of the vector, cells are allowed to grow in enriched media, and are then switched to selective media. The selectable marker confers resistance to the selective agent, and thus, only those cells that successfully express the introduced sequences survive in the selective media. Any number of selection systems may be used to recover transformed cell lines. These include, but are not limited to, the herpes simplex virus thymidine kinase and adenine phosphoribosyltransferase genes, for use in tk- or apr-cells, respectively (see e.g. Wigler, M. et al. (1977) “Transfer of purified herpes virus thymidine kinase gene to cultured mouse cells.” Cell 11:223-32; Lowy, I. et al. (1980) “Isolation of transforming DNA: cloning the hamster aprt gene.” Cell 22:817-23). Also antimetabolite, antibiotic, or herbicide resistance can be used as the basis for selection. For example, dhfr confers resistance to methotrexate,; neo confers resistance to the aminoglycosides, neomycin and G-418, and als and pat confer resistance to chlorsulfuron and phosphinotricin acetyltransferase, respectively. (see Wigler, M. et al. (1980) “Transformation of mammalian cells with an amplifiable dominant-acting gene.” PNAS 77:3567-70; Colbere-Garapin, F. et al (1981) “A new dominant hybrid selective marker for higher eukaryotic cells.” J. Mol. Biol. 150:1-14). Additional selectable genes have been described, e.g. trpB and hisD, which alter cellular requirements for metabolites. Visible markers, e.g. anthocyanins, green, red or blue fluorescent proteins (Clontech), B glucuronidase and its substrate B glucuronide, or luciferase and its substrate luciferin, may also be used. Resistant clones containing stably transformed cells may be propagated using tissue culture techniques appropriate to the cell type.

Host cells transformed/transfected with nucleotide sequences encoding for the perturbagen of interest may be cultured under conditions suitable for the expression and recovery of the protein from cell culture. For example, the protein produced by a transformed transfected cell may be secreted when the selected expression vector incorporates signal sequences that direct secretion of the perturbagen through a prokaryotic or eukaryotic cell membrane.

Signal sequences also may be selected so as to direct the perturbagen to a particular intra-cellular compartment (Bradshaw, R. A. (1989) “Protein translocation and turnover in eukaryotic cells.” Trends Biochem Sci 14(7):276-9). Perturbagen sequences may be isolated or purified from recombinant cell culture by methods heretofore employed for other proteins, e.g. native or reducing SDS gel electrophoresis, salt precipitation, isoelectric focusing, immobilized pH gradient electrophoresis, solvent fractionation, and chromatography such as ion exchange, gel filtration, immunoaffinity, and ligand affinity.

H. Host Cells

Host cell lines for use in the methodology described herein typically embody desirable traits such as 1) short cell cycle (i.e. 20-36 hr. doubling time), 2) amenability to high throughput procedures (e.g. FACS) without undue loss of membrane integrity or viability, 3) susceptibility to standard techniques designed to introduce reporter constructs and other forms of foreign DNA, and 4) exhibition of a readily selected phenotype (or its correlative marker gene expression). As one non-limiting example, the cell line is an ATRA-responsive subline of the melanoma cell line WM35. This WM35 subline is highly susceptible to retroviral infection and other methods of introducing foreign genetic materials and can express/maintain said materials for long periods of time using a variety of selectable markers common to the field (e.g. neomycin, puromycin). In addition, such sublines can be grown for limited periods of time (5-8 days) in media containing supplements (FBS) that have been depleted of small hydrophobic compounds (including ATRA and related small molecules) by chemical methods (e.g. charcoal stripping).

Reporter cell lines consist of a host cell that contains a reporter gene operably-linked to a cis-acting promoter that is related to the RA pathway. The terms “operably-associated” and “operably-linked” refer to functionally related nucleic acids. A promoter is “operably-associated” or “operably-linked” with a coding sequence if the promoter controls or regulates the transcription of the gene to which it is linked. Reporter cell lines can be obtained by several methodologies. For instance, episomal vectors carrying reporter constructs can be transformed into the cell type of choice and selected to identify rare events in which the reporter becomes stably incorporated into the genome. More preferably, a population of host cells containing the reporter construct of interest can be constructed using standard retroviral technology (Palu, G. et al. (2000) “Progress with retroviral gene vectors.” Rev Med Virol. 10(3):185-202). Preferred reporter lines exhibit several properties including 1) a large (>50-fold) “induced signal” to “uninduced signal” ratio, 2) a minimal number (<5%) of uninduced (“dim”) cells when the population has been fully induced and, 3) a minimal number (<5%) of induced (“bright”) cells when the populations is grown under non-inducing conditions.

One representative subline containing an ATRA-sensitive reporter is referred to herein as Clone 8. The reporter construct of Clone 8 consists of a retinoic acid response element (RARE) operably-linked in cis to a reporter gene (EGFP, Clontech). Variants of the RARE promoter sequence exist (Panariello, L. et al. (1996) “Identification of a novel retinoic acid response element in the promoter region of the retinol-binding protein gene.” J Biol Chem. 271(41):25524-32; de The, H. et al. (1990) “Identification of a retinoic acid responsive element in the retinoic acid receptor beta gene.” Nature 343(6254):177-80), and can be obtained from a variety of ATRA inducible genes including, but not limited to, RARα, RARβ, and RARγ genes. In addition, RARE promoter sequences need not be derived from natural sources but instead can be artificially constructed. Such artificial RARE sequences can exist as monomers of the RARE consensus sequence (A/G)G(G/T)TCANNNNN(A/G)G(G/T)TCA or can be repeated multiply in tandem. For this reason, the size of the ATRA responsive promoter fragment can vary considerably. Synthetic promoters, which often consist of tandem repeats of the RARE consensus sequence, can be relatively small (e.g. 17-51 nucleotides) while natural RARE promoters sequences can be considerably larger (e.g. >500 bp) depending upon the breakpoints used to isolate the element.

Applying the methodology described herein, one of ordinary skill in the art can readily screen for and identify individual ATRA-sensitive clones such as Clone 8. For example, to identify such sublines from the WM 35 melanoma line, one transfects a population of WM35 cells with the RARE-GFP retroviral construct and screens for individual members that are responsive to the presence of ATRA. The ability of WM35_{RARE-GFP} cells to respond to ATRA is influenced by several factors including, but not limited to, 1) the presence of needed host-encoded elements (e.g. RARα, RXR, necessary to detect the RA signal and 2) the site in which the RARE-GFP retroviral reporter construct is inserted into the WM35 genome. To isolate individual cells that exhibit a robust response to the presence of ATRA, a population of WM35_{RARE-GFP} cells (>100,000) are grown for 2-5 days in media containing either CBI serum (Cocalico Biological) supplemented with ATRA (100 nM-1 uM ATRA or related compounds, e.g. 9-cis-retinoic acid) or FBS serum that contains endogenous ATRA and ATRA derivatives. Subsequently, cells that express high levels of the reporter (GFP) are separated from non-or less-responsive members of the population by FACS. These cells are then expanded to sufficient numbers (>10⁶) prior to repeating the procedure for further enrichment. Following multiple cycles of the growth/sorting operation, the population is transferred to media that has been stripped of ATRA (e.g. CBI media without ATRA) and uninduced cells (“dims”) are collected. In this way, it is possible to separate cells that constitutively express the reporter gene due to, e.g., the effects of the genomic DNA flanking the reporter insertion site, from those that are truly responsive to ATRA. Subsequently, individual clones can then be examined for properties such as 1) level of ATRA-induced induction, 2) percent signal overlap between induced and uninduced populations and 3) overall sensitivity to ATRA. As one familiar with the art is aware, there are several variations to the procedures described above that could be used to achieve the same end results. For instance, FACS could be replaced with antibody affinity chromatography methods (using cell surface localized reporter) to isolate ATRA-responsive clones (see, for example, Larsson, P. H. et al. (1989) “Improved cell depletion in a panning technique using covalent binding of immunoglobulins to surface modified polystyrene dishes.” J Immunol Methods. 116(2):293-8; Contractor, S. F. et al. (1988) “Human placental cells in culture: a panning technique using a trophoblast-specific monoclonal antibody for cell separation.” J Dev Physiol. 10(1):47-51). Alternatively, the gates used to sort bright (or dim) cells in the FACS procedures could be adjusted to increase or decrease the enrichment procedures and thus alter the population of cells being collected and studied (see, for example Shapiro, H. M. (1995) “Practical Flow Cytometry” Wilely-Liss publishers). Furthermore, the protocol used in these proceedings (i.e. enriching for “bright” cells on three consecutive sorts, followed by identifying dim cells manually using a fluorescent microscope), could be changed without altering the ability to isolate ATRA responsive clones. For instance, in one non-limiting example, cells that constitutively express the reporter could be removed (by FACS) at that beginning of the procedure, followed by subsequent enrichment of cells that respond to the ATRA. The invention envisions each and every possible variation of these procedures that achieve the same end result.

As one of the art can appreciate, there are many other suitable host cell lines including, but not limited to, transformed and/or immortalized cell lines derived from (i) lung (Manna, S. K. et al. (2000), “All-trans-retinoic acid upregulates TNF receptors and potentiates TNF-induced activation of nuclear factors-kappaB, activated protein-1 and apoptosis in human lung cancer cells.” Oncogene 19(17):2110-9); (ii) breast or prostate (Koshiuka, K. et al. (2000), “Novel therapeutic approach: organic arsenical melarsoprol alone or with all-trans-retinoic acid markedly inhibit growth of human breast and prostate cancer cells in vitro and in vivo.” Br J Cancer 82(2):452-8; Baj, G. et al. (1999) “All-trans retinoic acid inhibits the growth of breast cancer cells by up-regulating ICAM-1 expression.” J Biol Regul Homeost Agents. 13(2):115-22); and (iii) ovaries (Jozan, S. et al. (1998), “Cytotoxic effect of interferon-alpha2a in combination with all-trans retinoic acid or cisplatin in human ovarian carcinoma cell lines.” Anticancer Drugs. 9(3):229-38). Any cell line such as these can be used as a host cell in the invention and can readily be screened to identify ATRA-responsive sublines.

I. Screening for Biological Activity

The phenotypic assay described herein selects for perturbagens that modulate the RA pathway. The procedures used to screen libraries for perturbagens include: 1) introducing perturbagen encoding sequences (libraries) into the reporter cell line and selecting stable integrants; 2) growing cells (containing both the reporter and one or more members of the library) under the appropriate conditions necessary to identify perturbagens that either stimulate or repress expression of the reporter; 3) screening cells by FACS or alternative high-throughput methods in order to segregate cells with the appropriate phenotype (e.g. “bright” or “dim”); 4) re-isolating perturbagen encoding sequences from sorted cell populations by various techniques (e.g. PCR); 5) enriching for perturbagens by recycling said sequences through the screen; and optionally 6) performing secondary assays to test specificity and scope of the agent. The methodology can identify perturbagens that either activate or inhibit the RA pathway. This is accomplished by placing the perturbagen library in an ATRA-responsive reporter cell population (e.g., a Clone 8 population) and raising the cells under one of two different conditions. Class I perturbagens (“dims”) represent perturbagens that disrupt the ATRA pathway and thus prevent the cell from transcribing the RARE-GFP reporter under conditions that normally induce reporter expression. In contrast, Class II perturbagens (“brights”) are those that activate the ATRA pathway under conditions that normally silence the reporter gene.

Various methods and instrumentation familiar to those who are skilled in the art are used to screen and test perturbagens. The media, supplements, and reagents used in culturing, packaging, and maintenance of WM35 cells, HS293gp packaging cell lines, and additional lines (e.g. HL60 (promyelocytic leukemia, ATCC: B1H1) can be purchased from a variety of commercial sources (Life Technologies, Clonetics, Cocalico Biologicals Inc.). It should be noted that although a particular set of procedures and media formulations are used in the work described herein, alternatives can be substituted with little or no effect. For instance, in most cases, retroviral packaging was accomplished using lipofectamine. Though this is the preferred method of introducing retroviral vectors into 293 gp packaging cells, alternative procedures such as the CaCl₂ method of packaging may be used. Molecular techniques used in procedures such as genomic DNA isolation, PCR amplification, DNA endonculease digestion, ligation, cloning, and sequencing utilize common reagents that are supplied commercially (see, for example, Qiagen, New England BioLabs, Stratagene).

Fluorescent activated cell sorting and analysis is performed on a Coulter EPICS Elite Cell Sorter using EXPO “Build” and EXPO “Analysis” software. Again, alternative reagents and equipment, such as the MOFlo^{R} High-Speed Cell Sorter (Cytomation), are compatible with these procedures and may be substituted with little or no effect.

To identify agents that activate the pathway, a library is introduced into, e.g., Clone 8 cells and grown in media that has been stripped of ATRA and related compounds. Under these conditions the vast majority of cells (99%+) fail to express the correlative reporter gene (e.g., a green, blue or red fluorescent protein). Subsequently, “bright” cells that potentially contain perturbagen(s) capable of activating the reporter in the absence of ATRA are collected by FACS. The perturbagen encoding sequences are then retrieved and recycled through the screen to enrich for sequences that activate the RARE-GFP reporter.

In another embodiment, the invention includes methods for screening for perturbagen clones that block or inactivate the retinoic acid pathway. To accomplish this, suitable RA-responsive host cells (e.g., Clone 8 cells) containing one or more members of a perturbagen library are grown under conditions that induce the expression of the reporter. Multiple conditions may be used to ensure reporter activation. For instance, Clone 8 cells can be grown in media supplemented with 1-2% fetal bovine serum (FBS) which contains endogenous ATRA (and ATRA-derivatives) capable of activating the RARE-fluorescent protein (FP) construct. Alternatively, Clone 8 cells can be grown in media containing roughly 1-2% charcoal-stripped FBS (CBI, Cocalico Biologics) and supplemented with 100 nM-1 uM ATRA (Sigma). Either protocol will ensure activation of the reporter in 95% or more of the Clone 8 cells. Upon activation of the Clone 8 population (24-48 hours of growth in ATRA⁺ media) “dim” cells are then sorted out in order to isolate perturbagens that disrupt or inhibit the activation of the RARE-FP reporter.

Biologically active perturbagen sequences are subsequently retrieved and then re-introduced into the screen to further enrich for perturbagens. Alternatively, the sequences can be re-introduced into a fresh Clone 8 population and subsequently plated at low density. After 8-10 days of growth in media containing ATRA, individual clones that fail to express the reporter, or express the reporter in only a fraction of the cells making up the clone, can be isolated. The perturbagen encoding sequence can then be re-isolated and tested for its ability to block RA activation of the RARE-FP reporter. As was the case in the previous embodiment, variations in these procedures and alternative sources of media/instrumentation can be substituted with minimal effects.

Several methods may be used to retrieve the perturbagen sequences from cells that have been sorted. For instance, perturbagen-encoding sequences may be recovered by PCR (see, for example, Schott, B. (1997) “Efficient recovery and regeneration of integrated retroviruses.” Nucleic Acids Res. 25(14):2940-2). To accomplish this, genomic DNA (derived from cells taken from the FACS sorting procedures is used as the template for PCR amplification. Using oligonucleotide primers that flank the perturbagen encoding sequence, complex mixtures with diversities of greater than 50,000 can be amplified efficiently. These sequences can subsequently be re-cloned into a retroviral vector, and introduced into a fresh population of, e.g., Clone 8 cells for additional rounds of screening. Alternatively, retrieval of the perturbagen may be accomplished by reactivating the inserted retroviral vector that contains the perturbagen-encoding sequence. Specifically, host cells containing the perturbagen-encoding (non-infective) retrovirus are transformed with sequences that encode the necessary retroviral gag, pol and envelope proteins. As a result of these procedures, infective retroviral virions that contain the perturbagen-encoding sequences are released and can be isolated in the form of a viral supernatant. These supernatants can then be used to infect fresh populations of, e.g., Clone 8 cells to recycle the sequences through the screen for additional enrichment.

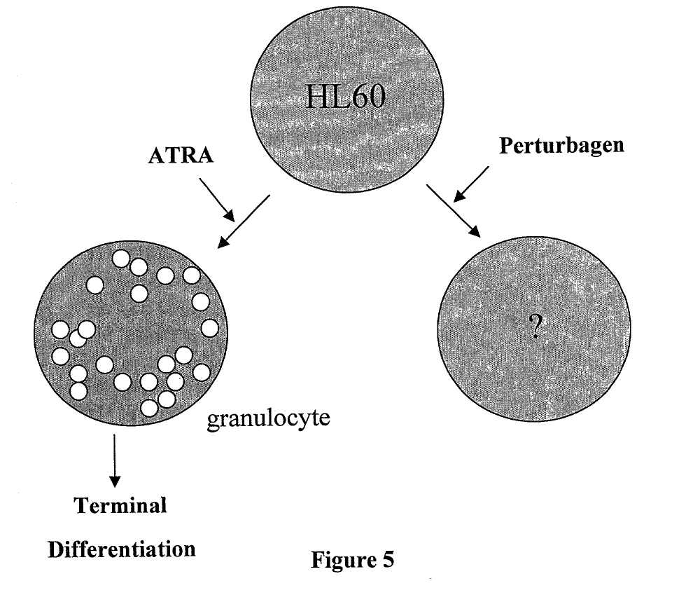

Secondary cell lines may optionally be employed to test individual perturbagens for the ability to induce an RA related phenotype. For example, previous studies have shown that when HL60 cells are exposed to ATRA, the cells undergo terminal differentiation. Thus, in order to test how closely each perturbagen mimics the action of ATRA, a secondary assay is developed to study whether perturbagens that induce transcription of the RARE-FP reporter in, e.g., Clone 8 cells can also induce differentiation of HL60 cells. To accomplish this, individual perturbagens that induce transcription of the RARE-FP reporter in the absence of ATRA, are introduced into the HL60 line under the control of an inducible promoter (e.g. the ecdysone or ponasterone-inducible promoter, PIND, Invitrogen). Upon exposure to ponasterone A, HL60-perturbagen-containing populations will be observed for indications that suggest they have undergone differentiation (FIG. 5).

J. Cellular Targets

In other embodiments, the invention encompasses the polypeptide, ribonucleotide, or polynucleotide sequence of the target (or fragment or variant of each target) that is identified with each perturbagen agent, as well as the gene encoding each target and relevant fragments of said gene.

Targets of specific perturbagens may be identified by several means. For instance, perturbagens can be modified with homo- or hetero- bifunctional coupling reagents and targets can be identified by chemical cross-linking techniques (see, for example, Tzeng, M. C. et al. (1995) “Binding proteins on synaptic membranes for crotoxin and taipoxin, two phospholipases A2 with neurotoxicity.” Toxicon. 33(4):451-7; Cochet, C. et al. (1988) “Demonstration of epidermal growth factor-induced receptor dimerization in living cells using a chemical covalent cross-linking agent.” J Biol Chem. 263(7):3290-5). Alternatively, one may use various techniques in column affinity chromatography, immunoprecipitation, or one of several high throughput peptide array platforms, to isolate peptides that react with the target of choice (see, for example, Hentz, N. G. and Daunert, S. (1996) “Bifunctional fusion proteins of calmodulin and protein A as affinity ligands in protein purification and in the study of protein-protein interactions.” Anal Chem. 68(22):3939-44; Figeys D and Pinto D. (2001) “Proteomics on a chip: promising developments.” Electrophoresis 22(2):208-16; Bichsel V. E. et al. “Cancer proteomics: from biomarker discovery to signal pathway profiling.” Cancer J 7(1):69-78). In some instances, a particular phenotype may be the result of a perturbagen differentially regulating a distinct combination of genes. For example, a perturbagen might, through its interaction with a particular transcription factor which, in turn, recognizes a particular DNA promoter sequence, elevate the expression of two or more target genes that act in concert to elicit a unique phenotype (e.g. viral resistance). In these cases, each of the genes whose levels of expression are altered by the perturbagen can be considered to be perturbagen targets. Such targets can be identified by a variety of techniques including (but not limited to) SAGE and expression profiling via microarray analysis (see, for instance, Cummings C. A. and Relman D. A. (2000) “Using DNA Microarrays to Study Host-Microbe Interactions.” Emerg Infect Dis. 6(5):513-525; Yamamoto M. et al. (2001) “Use of serial analysis of gene expression (SAGE) technology. J Immunol Methods. April 2001;250(1-2):45-66).

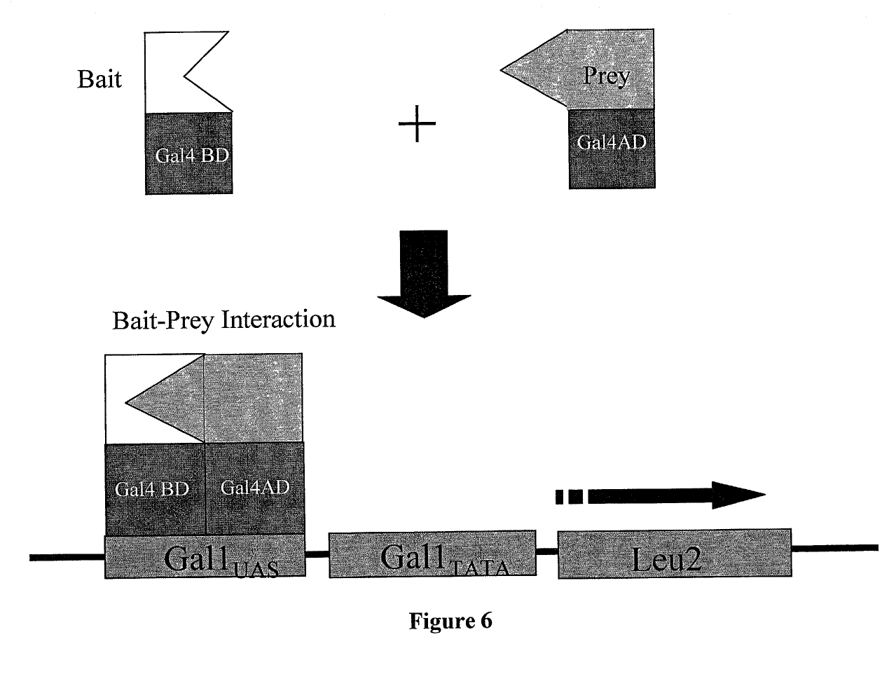

A preferred method of target identification involves application of variants of the standard two-hybrid technology. See, e.g., U.S. Ser. No. 09/193,759 and WO 00/29565 “Methods for validating polypeptide targets that correlate to cellular phenotypes”, the entire disclosures of which are incorporated by reference herein. Generally stated, the two-hybrid procedure is a quasi-genetic approach to detecting binding events. This assay often is performed in yeast cells (although it can be adapted for use in mammalian and bacterial cells), and relies upon constructing two vectors; the first having an interaction probe or bait (that in this case, will be the perturbagen) that typically is fused to a DNA binding domain (“BD”) moiety, and a second vector having an interaction target or prey (a cDNA library) that typically is fused to a DNA transcriptional moiety (the activation domain or “AD”). Neither of the two fusion proteins can, individually, induce transcription of the reporter gene. Yet when the bait and prey interact, the AD and BD moieties are brought into sufficient physical proximity to result in transcription of a reporter gene (e.g., the His3 gene or lacZ gene) located downstream of the bound complex (FIG. 6). Prey/bait interactions are then detected by identifying yeast cells that are expressing the reporter gene—e.g. which express lacZ or are able to grow in the absence of histidine.

A variety of yeast host strains known in the art are suitable for use for identifying targets of individual perturbagens. One of ordinary skill will appreciate that a number of factors may be considered in selecting suitable host strains, including but not limited to (1) whether the host cells can be mated to cells of opposite mating type (i.e., they are haploid), and (2) whether the host cells contain chromosomally integrated reporter constructs that can be used for selections or screens (e.g., His3 and LacZ). Although mating can be desirable in some embodiments, it is not strictly necessary for purposes of practicing the present invention. For example, the mating procedures can be eliminated by introducing the bait and prey constructs into a single yeast cell, whereupon the screens can be performed on the haploid cell.

Generally, either Gal4 strains or LexA host strains may be used with the appropriate reporter constructs. Representative examples include strains yVT 69, yVT 87, yVT96, yVT97, yVT98 and yVT99, yVT100, yVT360. Additionally, those of ordinary skill will appreciate that the host strains used in the present invention may be modified in other ways known to the art in order to optimize assay performance. For example, it may be desirable to modify the strains so that they contain alternative or additional reporter genes that respond to two-hybrid interactions.

The following host yeast strains are thus constructed to have the indicated characteristics:

YVT69: yVT69 (mat α, ura3-52, his3-200, ade2-101, trp1-901, leu2-3, 112, gal4Δ, met⁻, gal80Δ, URA3::GAL1_{UAS}-GAL1_{TATA}-lacZ) was obtained from Clontech (Y187).

YVT87: yVT87 (Mat-α ura3-52,his3-200,trp1-901,LexA_{op (x6)}-LEU2-3,112) was obtained from Clontech (EGY48).

YVT96: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT96, MATa ura3-52 his3-200 ade 2-101 ade5 lys2::GAL2-URA3 leu2-3, 112 trp1-901 tyr1-501 gal4D gal80Δ ade5::hisG by homologous recombination of Reporter 1 to the LYS2 locus. The integration is confirmed by PCR.

YVT97: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 will be converted to yVT97, MATα ura3-52 his3::GAL1 or GAL7-HIS3 ade2-101 ade5 lys2-801 leu2-3, 112 trp1-901 tyr1-501 gal4Δ gal80Δ ade5::hisG by the steps of (a) converting from MATa to MATα via transient expression of the HO endonuclease, Methods in Enzymology Vol. 194:132-146 (1991) and (b) integrating either of Reporters 3 or 4 at the HIS3 locus via homologous recombination. The integration is confirmed by PCR.

YVT98: The starting strain was EGY48 (Estojak, J. Et al., 1995) MATα, ura3 his3 trp1 leu2::LexAop(x6)-LEU2. EGY48 was converted to strain yVT98 MATα ura3 his3 trp1 leu2::lexAop(x6)-LEU2 lys2::lexAop(8x or 2x)-LacZ by homologous recombination of Reporter 6 into the LYS2 locus.

YVT99: The starting strain was EGY48 (Estojak, J. Et al., 1995) MATα, ura3 his3 trp1 leu2::LexAop(x6)-LEU2. EGY48 was converted to strain yVT99 MATa ura3 his3 trp1 leu2::lexAop(x6)-LEU2 lys2::lexAop(8x or 2x)-URA3 by homologous recombination of Reporter 2 into the LYS2 locus and by switching the mating type from MATα to MATa via transient expression of the HO endonuclease.

YVT100: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT100, MATa ura3-52 his3-200 ade2-101 ade5 lys2::lexAop(8x or 2x)-URA3 leu2-3, 112 trp1-901 tyr-501 gal4Δ gal80Δ ade5::hisG by homologous recombination of Reporter 2 to the LYS2 locus. The integration was confirmed by PCR.

YVT360: yVT360 (mat a, trp1-901, leu2-3,112, ura3-52, his3-200, gal4•, gal 80•, LYS2::GAL1_{UAS}-GAL1_{TATA}-HIS3, GAL2_{UAS}-GAL2_{TATA}-ADE2, URA3:MEL1_{UAS}-MEL1_{TATA}-lacZ) was obtained from Clontech (AH109).

Exemplary yeast-reporter strains are constructed using a variety of standard techniques. Many of the starting yeast strains already carry multiple mutations that lead to an auxotrophic phenotype (e.g. ura3-52, ade2-101). When necessary, reporter constructs can be integrated into the genome of the appropriate strain by homologous recombination. Successful integration can be confirmed by PCR. Alternatively, reporters may be maintained in the cells episomally.

The yeast two-hybrid reporter gene typically is fused to an upstream promoter region that is recognized by the BD, and is selected to provide a marker that facilitates screening. Examples include the lacZ gene fused to the Gal1 promoter region and the His3 yeast gene fused to Gal1 promoter region. A variety of yeast two-hybrid reporter constructs are suitable for use in the present invention. One of ordinary skill will appreciate that a number of factors may be considered in selecting suitable reporters, including whether (1) the reporter construct provides a rigorous selection (i.e., yeast cells die in the absence of a protein-protein or peptide-protein interaction between the bait and prey sequences), and/or (2) the reporter construct provides a convenient screen (e.g., the cells turn color when they harbor bait and prey sequences that interact). Examples of desirable reporters include (1) the Ura3 gene, which confers growth in the absence of uracil and death in the presence of 5-fluoroorotic acid (5-FOA); (2) the His3 gene, which permits growth in the absence of histidine; (3) the LacZ gene, which is monitored by a colorimetric assay in the presence/absence of beta-galactosidase substrates (e.g. X-gal); (4) the Leu2 gene, which confers growth in the absence of leucine; and (5) the Lys2 gene, which confers growth in the absence of lysine or, in the alternative, death in the presence of α-aminoadipic acid. These reporter genes may be placed under the transcriptional control of any one of a number of suitable cis-regulatory elements, including for example the Gal2 promoter, the Gal1 promoter, the Gal7 promoter, or the LexA operator sequences.

The following are exemplary, non-limiting examples of such reporter constructs.

Reporter 1—(pVT85): This reporter comprises the URA3 gene under the transcriptional control of the yeast Gal2 upstream activating sequence (UAS). In order to facilitate integration of this reporter into the yeast chromosome in place of the Lys2 coding region, the Gal2-Ura3 construct is flanked on the 5′ side by the 500 base pairs that lie immediately upstream of the coding region of the LYS2 gene and on the 3′ side by the 500 base pairs that lie immediately 3′ of the coding region of the LYS2 gene. The entire vector is also cloned into the yeast centromere containing vector pRS413 (Sikorski, RS and Hieter, P., Genetics 122(1):19-27 (1989) and can therefore be used episomally. This reporter is intended for use with a Gal4-based two-hybrid system, e.g., Fields, S. and Song, O., Nature 340:245-246 (1989).

Reporter 2—(pVT86): This reporter is identical to reporter #1 except that the GAL2 UAS sequences have been replaced with regulatory promoter sequences that contain eight LexA operator sequences (Ebina et al., 1983). The number of LexA operator sequences in this reporter may either be increased or decreased in order to obtain the optimal level of transcriptional regulation. This reporter is intended to be used within the general confines of the LexA-based interaction trap devised by Brent and Ptashne.

Reporter 3—(pVT87): This reporter is comprised of the yeast His3 gene under the transcriptional control of the yeast Gal1 upstream activating sequence (UAS). In order to facilitate integration of this reporter into the yeast chromosome in place of the His3 coding region the Gal1-His3 construct is flanked on the 5′ side by the 500 base pairs (bp) immediately upstream of the His3 coding region and on the 3′ side by the 500 bp immediately 3′ of the His3 coding region. The entire reporter is also cloned into the yeast centromere containing vector pRS415 and can therefore be used episomally. This reporter is intended for use with a Gal4-based two-hybrid system.

Reporter 4—(pVT88): This reporter is identical to Reporter 3 except that the His3 gene is under the transcriptional control of Gal7 UAS sequences rather than the Gal1 UAS. The reporter is used with a Gal4-based two-hybrid system.

Reporter 5—(pVT89): This reporter contains the bacterial LacZ gene under the transcriptional control of the Gal1 UAS. The entire reporter will be cloned into a yeast centromere-using vector, e.g., pRS413, and is used episomally.

Reporter 6—(pVT90): This reporter consists of the LacZ gene under the transcriptional control of eight LexA operator sequences. As for Reporter 2, the number of LexA operator sequences in this reporter may either be increased or decreased in order to obtain optimal levels of transcriptional regulation. Two features of this reporter facilitate integration of the reporter into the yeast chromosome in place of the Lys2 coding region. First, it is flanked on the 5′ side by the 500 base pairs that lie immediately upstream of the coding region of the Lys2 gene and on the 3′ side by the 500 base pairs that lie immediately 3′ of the coding region of the Lys2 gene. Second, the neomycin (NEO) resistance gene has been inserted between the 5′ Lys2 sequences and the LexA promoter sequences. This reporter is used in conjunction with a LexA-based interaction trap, e.g., Golemis, E. A., et al., (1996), “Interaction trap/two hybrid system to identify interacting proteins.” Current Protocols in Molecular Biology, Ausebel et al., eds., New York, John Wiley & Sons, Chap. 20.1.1-20.1.28.

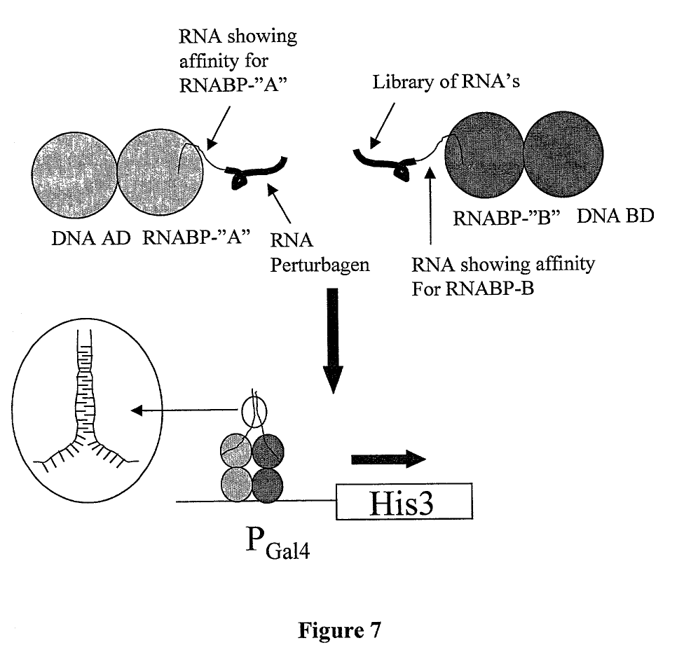

In other embodiments, perturbagen-induced phenotypes may be the result of RNA-RNA, RNA-polypeptide, polypeptide-DNA, or RNA-DNA interactions. In cases such as these, variations of the original two-hybrid theme may be applied to identify the target of the phenotypic probe. (See, for example, Li, J. J. and Herskowitz, I. (1993) Isolation of Orc6, a Component of the Yeast Origin Recognition Complex by a One-Hybrid System. Science, 262:1870-1874; Svinarchuk, F. et al. (1997) “Recruitent of transcription factors to the target site by triplex-forming oligonucleotides.” NAR 25: 3459-3464; Segupta, D. J. et al. (1999) “Identification of RNAs that bind to a specific protein using the yeast three-hybrid system.” RNA 5:596-601; Harada, K. et al. (1996) “Selection of RNA-binding peptides in vivo.” Nature 14;380(6570):175-9; SenGupta, D. J. et al. (1996) “A three-hybrid system to detect RNA protein interactions in vivo.” PNAS 93:8496-8501). For instance, if evidence exists that a perturbagen is acting as an anti-sense agent, it is necessary to construct a system where the association of the DNA binding domains and the transcriptional activation domains is dependent upon and RNA-RNA interaction. To accomplish such a screen, four unique vectors are created (FIG. 7). The first vector consists of the DNABP (e.g. GAL4 BD) described previously, linked to a specific RNA binding protein, arbitrarily called “RNABP-A” (e.g. the Rev responsive element RNA binding protein, RevM10, see Putz, U. et al. (1996) “A tri-hybrid system for the analysis and detection of RNA-protein interactions.” NAR 24:4838-4840). Vector #2 contains the transcriptional activation domain (e.g. GAL4 AD) linked to a second RNA binding protein (“RNABP-B”, e.g. the MS2 coat protein of the MS2 bacteriophage, see for example, SenGupta, D. J. et al. (1996) “A three hybrid system to detect RNA-protein interactions in vivo.” PNAS 93:8496-8501). The third vector encodes an RNA molecule that is recognized by RNABP-A (e.g. the RRE sequence, Zapp, M. L. and Green M. R./“Sequence-specific RNA binding by the HIV-1 Rev protein (1989) Nature, 32:714-716) fused to a sequence encoding the RNA perturbagen, while the final vector encodes a fourth hybrid, the RNA sequence recognized by RNABP-B (e.g. the 21 base nucleotide RNA stem-loop structure of MS2, see Uhlenbeck, O. C. et. al. (1983) “Interaction of R17 coat protein with its RNA binding site for translational repression.” J. Biomol Struct. Dyn. 1, 539-552) linked to a library of expressed sequences (e.g. a library of mRNA molecules). When all four vectors are stably maintained in a yeast cell containing the necessary reporter construct(s) (e.g. P_{GAL4}-LACZ), the cellular target RNA molecule of any given RNA perturbagen can be identified.

Target sequences or fragments thereof can vary greatly in size. Some target fragments can be as small as ten amino acids in length. Alternatively, target sequences can be greater than 10 amino acids but less than thirty amino acids in length. Still other targets can be greater than thirty amino acids in length but shorter than 60 amino acids in length. Still other targets are cellular proteins or subunits or domains therein of more than 60 amino acids in length. Still other targets are cellular proteins or subunits or domains there of more than 60 amino acids in length. Still other targets are cellular proteins or subunits or domains there of more than 60 amino acids in length. In addition, for reasons described previously, the sequences encoding targets can vary greatly due to allelic variation, duplications and closely related gene family members. That said, the invention also encompasses variants of said targets. A preferred target variant is one which has at least about 80%, alternatively at least about 90%, and in another alternative at least about 95% amino acid sequence identity to the original target amino acid sequence and which contains at least one functional or structural characteristic of the original target.

K. Databases

The compositions, relations and phenotypic effects yielded by the methodology described herein may advantageously be placed into or stored in a variety of databases. As one example, a database may include information about one or more targets identified by the methods herein, including for example sequence information, motif information, structural information and/or homology information. The database may optionally contain such information regarding perturbagen agents, and may correlate the perturbagen information to corresponding target information. Further helpful database aspects may include information regarding, e.g., variants or fragments of the above. The database may also correlate the indexed compounds to, e.g., immunoprecipitation data, further yeast n-hybrid interaction data, genotypic data (e.g., identification of disrupted genes or gene variants), and with a variety phenotypic data. Such databases are preferably electronic, and may additionally be combined with a search tool so that the database is searchable.

L. Production of Antibodies

An additional embodiment of the invention includes antibodies that recognize the perturbagen itself, cellular targets of the perturbagen, or one or more epitopes of the foregoing. Such reagents may include, but are not limited to, polyclonal, monoclonal, humanized, chimeric, and single chain antibodies, Fab fragments, F(ab′)₂ fragments, fragments produced by a Fab expression library, anti-idiotypic (anti-Id) antibodies, and epitope-binding fragments of any of the above. Antibodies directed against perturbagens or cellular targets may be useful for a variety of purposes including i) therapeutics, ii) diagnostic assays, iii) cytoimmunology, iv) target identification, and v) purification.

For the production of antibodies, various hosts including goats, rabbits, rats, mice, humans and others may be immunized by injection with a perturbagen, target or any fragment thereof which has immunogenic properties. Depending on the host species, various adjuvants may be used to increase immunological response. Such adjuvants include, but are not limited to Freund's (complete and incomplete), mineral gels such as aluminum hydroxide, and surface-active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Polyclonal antibodies are heterogeneous populations of antibody molecules derived from the sera of animals immunized with an antigen, such as a given perturbagen, target, or an antigenic functional derivative thereof. For the production of polyclonal antibodies, host animals such as those described above, may be immunized by injection with gene product supplemented with adjuvants as also described above.

Monoclonal antibodies that recognize perturbagens may be prepared using any technique that provides for the production of antibody molecules by continuous cell lines in culture. These include, but are not limited to, the hybridoma technique, the human B-cell hybridoma technique, and the EBV hybridoma technique. (see, for example, Kohler, G. et al. (1975) “Continuous cultures of fused cells secreting antibody of predefined specificity.” Nature 256:495-497; Kozbor, D. et al (1985) “Specific immunoglobulin production and enhanced tumorigenicity following ascites growth of human hybridomas.” J. Immunol. Methods 81:31-42; Cote, R. J. et al. (1983) PNAS 80:2026-2030; and Cole, S. P. et al. (1984) “Generation of human monoclonal antibodies reactive with cellular antigens” Mol. Cell Biol. 62:109-120).

In addition, one may use techniques developed for the production of chimeric antibodies, such as the splicing of mouse antibody genes to human antibody genes to obtain a molecule with appropriate antigen specificity and biological activity. See, e.g., Morrison, S. L. et al. (1984) “Chimeric human antibody molecules: mouse antigen-binding domains with human constant region domains.” PNAS 81:6851-6855); Neuberger, M. S. et al. (1984) “Recombinant antibodies possessing novel effector functions.” Nature 312:604-608; and Takeda, S. et al. (1985) “Construction of chimeric processed immunoglobulin genes containing mouse variable and human constant region sequences.” Nature 314:452-454). Alternatively, techniques described for the production of single chain antibodies may be adapted, using methods known in the art, to produce perturbagen-specific antibodies (see, e.g. Burton, D. R. (1991) “A large array of human monoclonal antibodies to type 1 human immunodeficiency virus from combinatorial libraries of asymptomatic seropositive individuals.” PNAS 88:10134-10137).

Antibodies may also be produced by inducing in vivo production in the lymphocyte population or by screening immunoglobulin libraries or panels of highly specific binding reagents as disclosed in the literature. (see, for example, Orlandi, R. et al. (1989) “Cloning immunoglobulin variable domains for expression by the polymerase chain reaction.” PNAS 86:3833-3837; Winter, G. et al. (1991) “Man-made antibodies.” Nature 349:293-299).

Antibody fragments that contain specific binding sites for perturbagens may also be generated. For example, such fragments include, but are not limited to F(ab′)₂ fragments produced by pepsin digesting of the antibody molecule and Fab fragments generated by reducing the disulfide bridges of the F(ab′)₂ fragments. Alternatively, Fab expression libraries may be constructed to allow rapid and easy identification of monclonal Fab fragments with the desired specificity. (See, for example, Huse, W. D. et al. (1989) “Generation of a large combinatorial library of the immunoglobulin repertoire in phage lambda.” Science 246:1275-1281).

M. Screening Assays

The agents of the invention can be used to screen for drugs or compounds (small molecules) that mimic or modulate the activity or expression of said phenotypic probes. The present invention may be employed in a process for screening for agents such as agonists, i.e. agents that bind to and activate an RA pathway target, or antagonists, i.e. inhibit the activity or interaction of an RA pathway target with an endogenous or exogenous ligand. Thus, polypeptides of the invention may also be used to assess the binding of small molecule substrates and ligands in, for example, cells, cell-free preparations, chemical libraries, and natural product mixtures as known in the art. Any methods routinely used to identify and screen for agents that can modulate receptors may be used in accordance with the present invention.

Like the perturbagen itself, such small molecule compounds may be used to treat disorders characterized by insufficient or excessive production of a target which has decreased or aberrant activity compared to the wild type entity. Thus, the invention provides a method for identifying modulators, i.e. candidate or test compounds or agents (e.g. peptidomimetics, small molecules or other drugs) that bind to the agent or its target, and have a stimulatory or inhibitory effect on the pathway(s) affected by said agent.

In vitro systems may be designed to identify compounds capable of binding, e.g., an RA pathway target gene product. Such compounds may include, but are not limited to, peptides made of D-and/or L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of RA pathway target gene proteins, preferably mutant proteins; elaborating the biological function of the RA pathway target gene protein; or screening for compounds that disrupt normal RA pathway target gene interactions or themselves disrupt such interactions.

In one embodiment, the invention provides libraries of test compounds. The test compounds of the present invention can be obtained using any of the numerous approaches in combinatorial library methods known in the art, including: biological libraries, spatially addressable parallel solid phase or solution phase libraries; synthetic library methods requiring deconvolution; the one-bead one-compound library method; and synthetic library methods using affinity chromatography selection. The biological library approach is exemplified by peptide libraries, while the other four approaches are applicable to peptide, non-peptide oligomer or small molecule libraries of compounds (Lam, K. S. (1997) “Application of combinatorial library methods in cancer research and drug discovery.” Anticancer Drug Des. 12:145).

Methods for the synthesis of molecular libraries can be found in the art, for example, in (i) De Witt, S. H. et al. (1993) “Diversomers: an approach to nonpeptide, nonoligomeric chemical diversity.” PNAS 90:6909, (ii) Erb, E. et al. (1994) “Recursive deconvolution of combinatorial chemical libraries.” PNAS 91:11422, (iii) Zuckermann, R. N. et al. (1994) “Discovery of nanomolar ligands for 7-transmembrane G-protein-coupled receptors from a diverse N-(substituted)glycine peptoid library.” J. Med Chem. 37:2678 and (iv) Cho, C. Y. et al. (1993) “An unnatural biopolymer.” Science 261:1303. Libraries of compounds may be presented in i) solution (e.g. Houghten, R. A. (1992) “The use of synthetic peptide combinatorial libraries for the identification of bioactive peptides.” BioTechniques 13:412) ii) on beads (Lam, K. S. (1991) “A new type of synthetic peptide library for identifying ligand-binding activity.” Nature 354:82), iii) chips (Fodor, S. P. (1993) “Multiplexed biochemical assays with biological chips.” Nature 364:555), iv) bacteria (U.S. Pat. No. 5,223,409), v) spores (U.S. Pat. Nos. 5,571,698, 5,403,484, and 5,223,409), vi) plasmids (Cull, M. G. et al. (1992) “Screening for receptor ligands using large libraries of peptides linked to the C terminus of the lac repressor.” PNAS 89:1865) or vii) phage (Scott, J. K. and Smith, G. P. (1990) “Searching for peptide ligands with an epitope library.” Science 249:386)

There are several methods for identifying small molecule compounds that mimic the action of the phenotypic probes. In one approach, an assay may be devised to directly identify agents that bind to, e.g., an RA pathway target protein. Such direct binding assays generally involve preparing a reaction mixture of the RA pathway target protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the RA pathway target protein or the test substance onto a solid phase and detecting target protein/test substance complexes anchored on the solid phase at the end of the reaction. In one embodiment of such a method, the RA pathway target protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for an RA pathway gene product or the test compound to anchor any complexes formed in solution, and a labeled antibody specific for the other component of the possible complex to detect anchored complexes.

Compounds that are shown to bind to a particular RA pathway gene product through one of the methods described above can be further tested for their ability to elicit a biochemical response from the RA pathway gene protein. Agonists, antagonists and/or inhibitors of the expression product can be identified utilizing assays well known in the art.

In another approach, perturbagen/target pairs are used to identify small molecule mimetics in a displacement assay format. Such assays can be based upon a variety of technologies including, but not limited to i) ELISAs (see, for example, Rice, J. W. et al. (1996) “Development of a high volume screen to identify inhibitors of endothelial cell activation.” Anal Biochem 241(2):254-9), ii) scintillation proximity assays (see, for example, Lerner, C. G. and Saiki, A. Y. C. (1996) “Scintillation proximity assay for human DNA topoisomerase I using recombinant biotinyl-fusion protein produced in baculovirus-infected insect cells.” Anal Biochem 240(2):185-96) or iii) time-resolved fluorescence resonance energy transfer-based technology (see, for example, Fernandes, P. B. (1998) “Technological advances in high-throughput screening.” Curr Opin Chem Biol 2(5):597-603; Hemmilä, “Time-resolved fluorometry—advantages and potentials in high throughput screening assays.” “High Throughput Screening”, J. Devlin (ed.). Marcel Dekker Inc, New York, pp. 361-76 (1997)). Two non-limiting examples of such assays, one homogeneous, LANCE™ (Stenroos, K. et al. (1997) “Homogeneous time resolved fluo-rescence energy transfer assay (LANCE) for the determination of IL-2-IL-2 receptor interaction.” Abstract of Papers Presented at the 3rd Annual Conference of the Society for Biomolecular Screening, Sep., California), and one heterogeneous, DELFIA™ (MacGregor, I. et al. (1999) “Application of a time-resolved fluoroimmunoassay for the analysis of normal prion protein in human blood and its components.” Vox Sang 77(2):88-96; Jensen, P. E. et al. (1998) “A europium fluoroimmunoassay for measuring peptide binding to MHC class I molecules.” J. Immunol. Methods 215:71-80; Takeuchi, T. et al. (1995) “Nonisotopic receptor assay for benzodiazepine drugs using time-resolved fluorometry.” Anal. Chem. 67: 2655-8.) are described as follows.

1. Lance™: Homogeneous Assay

To identify small molecules capable of disrupting the interaction between the perturbagen and its target, assays are designed to utilize the LANCE™ technology (commercially available from E. G. & G. Wallac.). LANCE™ is a homogeneous assay that is performed in solution and requires no wash steps to separate bound and unbound label. Briefly, the target is produced in large quantities and labeled with a lanthanide chelate (i.e. a fluorescent donor such as a Europium, (Eu) or Terbium (Tb) chelate). Concomitantly, the perturbagen is labeled with one of several fluorescent “acceptor” moieties that can be excited by the emissions of the donor molecule (e.g. allophycocyanin (APC) or rhodamine Rh, respectively). Most preferably, 1) the modification of either the perturbagen or the target is not detrimental to the interaction between the two interacting molecules being studied and 2) the distance separating the donor and acceptor moieties when the perturbagen and the target are associated, is sufficiently close to permit FRET (typically 30-100 Angstroms). As an alternative to direct labeling of the perturbagen, monoclonal antibodies directed against the perturbagen can be labeled with Eu, thus allowing small molecule displacement assays to take place via indirect labeling procedures.

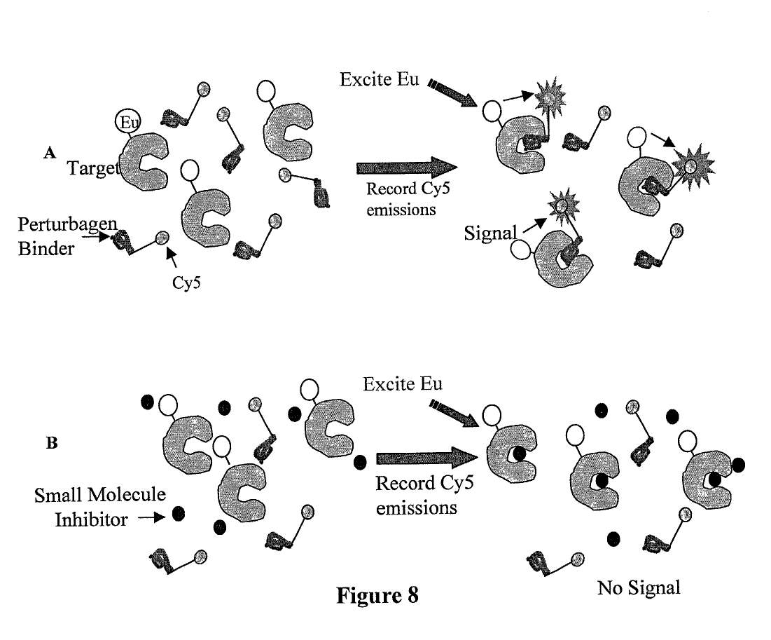

To identify small molecules capable of disrupting the interaction between the perturbagen and its target, the two labeled components are alliquoted into wells (1536 well format) at previously set, optimized conditions that will ensure 50% binding (FIG. 8). Subsequently, each well is then exposed to one or more members of a large chemical combinatorial library and time-resolved measurements are taken using a Wallac 1420 Victor multilabel counter or equivalent fluoremeter. In wells that contain a small molecule that interferes with the interaction between the perturbagen and its target, the distance separating the donor and acceptor molecules is increased. As a result of this dissociation or displacement, the ability of the Eu emissions to excite the acceptor is compromised and the total fluorescence emitted by the acceptor is decreased.

2. Heterogeneous Assay: DELFIA™

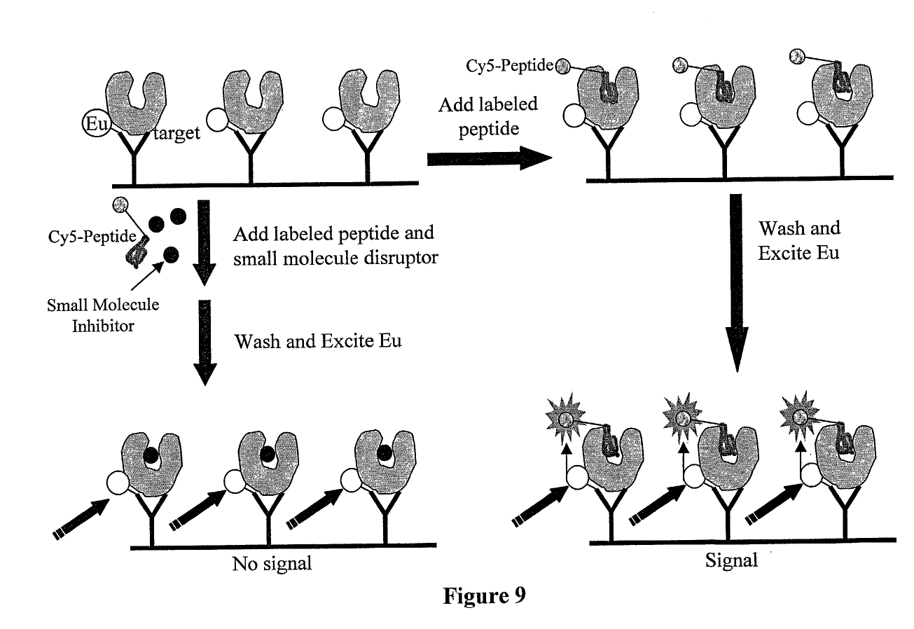

Several variations of a heterogeneous assay (DELFIA™) using an immobilized substrate can be used as an alternative to LANCE™. In one non-limiting example, the target is immobilized to a solid support using a monoclonal antibody that has been labeled with Eu (FIG. 9). Subsequent addition and binding of a rhodamine labeled perturbagen in the presence or absence of a candidate small organic displacement molecule is followed by several wash steps to remove unbound material. TR-FRET is then performed by exciting Eu and measuring the levels of Rh emissions. As an alternative to this procedure, the target is immobilized to the solid support using an unlabeled monoclonal antibody. Subsequently, an Eu-labeled perturbagen (+/− a candidate small organic displacement molecule) is added to each well and allowed to equilibrate, followed by a washing procedure to eliminate unbound Eu-labeled material. Once the well has been cleared of all unbound material, the bound Eu-perturbagen molecules are released and excited in the presence of commercially available enhancement solutions (DELFIA™ Enhancement Solutions, Wallac). By comparing the levels of emissions in wells that contain members of the molecule library with standardized controls, small molecules that disrupt the interaction between the perturbagen and its target are identified.

Another preferred method for identifying small molecule mimetics makes use of a variation of the two-hybrid technology. As one non-limiting example of how a two-hybrid chemical screen is performed, the yeast host cells containing i) AD-perturbagen, ii) the BD-target, and iii) a reporter construct made up of a promoter recognized by the BD, functionally linked to, for instance, the gene encoding lacZ or ZsGreen (Clontech), are grown in liquid culture media and subjected to the test chemical. Assay plates are then incubated at 30° C. for 48 hours and samples are scored by looking the expression of the marker by FACS or other conventional techniques. As an alternative, compounds that are attached to a solid support (e.g. beads) can be tested for their ability to rescue the growth phenotype in solution-based assays. Specifically, yeast cells modified for reverse genetic studies can be arrayed in nanodroplets (100-200 nanoliter volumes) that contain i) the selective elements of the medium (e.g. 5-FOA, cyclohexamide) and ii) one or more beads linked to a chemical library member. Subsequent photolysis of the chemical agent from the bead allows diffusion of the test molecules into the yeast cell and disruption of the two-hybrid interaction (see, Borchardt A et al. (1997) “Small molecule-dependent genetic selection in stochastic nanodroplets as a means of detecting protein-ligand interactions on a large scale.” Chemical Biology 4(12):961-8; You, A. J. et al. (1997) “A miniaturized arrayed assay format for detecting small molecule-protein interactions in cells.” Chem Biol. 4(12):969-75; Huang, J. Schreiber, S. L. (1997) “A yeast genetic system for selecting small molecule inhibitors of protein-protein interactions in nanodroplets.” PNAS 94:13396-13401; Young, K. et al. (1998), “Identification of a calcium channel modulator using a high throughput yeast two-hybrid screen.” Nature Biotechnology 16:946-950).

L. Therapeutic Uses

ATRA or ATRA derivatives have proven valuable in the treatment of a variety of diseases. For that reason, in one embodiment, perturbagens, fragments or derivatives of a perturbagen, small molecule mimetics of a perturbagen, sequences encoding perturbagens, sequences that can hybridize to perturbagen encoding sequences, RA pathway targets of the perturbagen, or agents that bind said target (e.g. antibodies) or portions thereof, may be utilized to treat or prevent a disorder that has previously shown sensitivity to treatment with retinoids. Thus, for example, polypeptides or RNA molecules described herein can be used i) modulate cellular proliferation, ii) modulate cellular differentiation, iii) induce or modulate necrotic or apototic processes, or iv) sensitize cells to secondary compounds that induce either i), ii), or iii) by direct application of said agent. Examples of such disorders that may be aided by such agents include, but are not limited to cancers of the i) head and neck, ii) small cell lung carcinoma, iii) hepatocellular carcinomas (HCC), iv) cancers of the breast, v) leukemias, vi) cutaneous T-cell lymphoma (CTCL), vii) non-small cell lung carcinoma, viii) neuroblastomas, ix) pancreatic carcinomas, x) Karposi's sarcoma, xi)renal cell carcinoma (RCC), xii) squamous cell carcinomas, and cancers of the xiii) prostrate, xiv) ovaries, and xv) cervix. In addition, retinoid therapy has proven effective in the treatment of a number of skin disorders. Thus, any of the agents of the invention may be administered to a subject to treat or prevent i) psoriasis, ii) hyperkeratosis, iii) eczema, iv) Multicentric Castlemans disease, v) pyoderma faciate, vi) acne vulgaris, vii) Darier's disease, viii) Reiter's disease, ix) follicular mucinosis, and other forms of dermatitis.

Ailments that respond to retinoid therapy can be treated with the perturbagen or RA pathway target directly, for example by administering a therapeutically effective dose of a proteinaceous agent intravenously or by other peptide delivery techniques known to the art. A therapeutically effective dose of a pharmaceutical composition comprising a substantially purified perturbagen, or a fragment thereof, or a small molecule mimetic, optionally in conjunction with a suitable pharmaceutical carrier, may be administered to a subject to treat or prevent a disorder previously shown to be sensitive to retinoid therapy. A “therapeutically effective” dose refers to that amount of the compound sufficient to result in amelioration of symptoms of the disease. A “pharmaceutical carrier” includes any and all solvents, dispersion media, coatings, antibacterial and antifungal agents, and the like, compatible with pharmaceutical administration. The use of such media and agents for pharmaceutically active substances is well known in the art. Except insofar as any conventional media or agent is incompatible with the active compound, use thereof in the compositions is contemplated.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions of the invention are formulated to be compatible with intended routes of delivery. Examples of routes of administration include parenteral e.g. intravenous, intradermal, subcutaneous, oral, inhalation, transdermal, topical, transmucosal, and rectal administration. Solutions or suspensions used for parenteral, intradermal, or subcutaneous application can include the following components: a sterile diluent, such as water for injection, saline solution, fixed oils, polyethylene, glycols, glycerine, propylene glycol, or other synthetic solvents, antibacterial agents such as benzyl alcohol or methyl parabens, antioxidants such as ascorbic acid or sodium bisulfite, chelating agents such as ethylenediaminetetraacetic acid, buffers such as acetates, citrates, or phosphates and agents for the adjustment of tonicity such as sodium chloride or dextrose.

Pharmaceutical compositions suitable for injectable use include aqueous solutions (where water-soluble) or dispersions and sterile powders for the extemporaneous preparation of sterile injectable solutions or dispersions. For intravenous administration, suitable carriers include physiological saline, bacteriostatic water Cremophor EL™ (BASF; Parsippany, N.J.) or phosphate buffered saline (PBS). In all cases the composition must be sterile and should be fluid to the extent that easy syringability exists. Oral compositions can also be prepared using any of the following ingredients, or compounds of a similar nature: a binder such as microcrystalline cellulose, gum tragacanth, or gelatin; an excipient such as starch or lactose, disintegrating agent such as alginic acid, Primogel, or corn starch; a lubricant such as magnesium stearate, a glidant such as colloidal silicon dioxide, a sweetening agent such as sucrose or saccharin, or a flavoring agent such as peppermint or orange flavoring. For administration by inhalation, the compounds are delivered in the form of an aerosol spray from a pressurized container or dispenser that contains a suitable propellant. Systemic administration can also be by transmucosal or transdermal means. For these methods of administration, penetrants appropriate to the barrier to be permeated are used in the formulation. Such penetrants are generally known in the art and include, for example, bile salts and fusidic acid derivatives. Transmucosal administration can also be accomplished through the use of nasal sprays and suppositories. For transdermal administration, the active compounds are formulated into ointments, salves, gels, or creams as generally known in the art.

In one embodiment, the active compounds are prepared with carriers that will protect the compound against rapid elimination from the body, such as a controlled microencapsulated delivery system. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to specific cell surface epitopes) can also be used as pharmaceutically acceptable carriers. These can be prepared according to methods known to those skilled in the art, for example, as described in U.S. Pat. No. 4,522,811.

Alternatively, such therapeutics can be administered indirectly, for example by gene therapy utilizing a gene or RNA sequence encoding a perturbagen, RA pathway target, or variant or fragment of the foregoing. For example, a vector capable of expressing a perturbagen or target, or a fragment or derivative thereof, may be administered to a subject to treat or prevent a disease. Expression vectors including, but not limited to, those derived from retroviruses, adenoviruses, adeno-associated viruses, or herpes or vaccinia viruses or from various bacterial plasmids, may be used for delivery of nucleotide sequences to the targeted organ, tissue, or cell population (see, for example, Carter, P. J. and Samulski, R. J. (2000) “Adeno-associated viral vectors as gene delivery vehicles.” Int J Mol Med. 6(1):17-27; Palu, G. et al. (2000) “Progress with retroviral gene vectors.” Rev Med Virol. 10(3):185-202; Wu, N. and Ataai, M. M. (2000) “Production of viral vectors for gene therapy applications.” Curr Opin Biotechnol. 11(2):205-8). Gene therapy vectors can be delivered to a subject by, for example, intravenous injection, local administration (U.S. Pat. No. 5,328,470) or by stereotactic injection (see, for example, Chen, S. H. et al. (1994) “Gene therapy for brain tumors: regression of experimental gliomas by adenovirus-mediated gene transfer in vivo.” PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

M. Antisense, Ribozyme and Antibody Therapeutics

Other agents that may be used as therapeutics include any RA pathway target genes, associated expression product and functional fragments thereof. Additionally, agents that reduce or inhibit mutant RA pathway target gene activity may be used to ameliorate disease symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the RA pathway target gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to a RA pathway target gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding anaphylatoxin C3a receptor gene proteins.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the RA pathway target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant RA pathway target gene alleles. In order to ensure that substantially normal levels of RA pathway target gene activity are maintained, nucleic acid molecules that encode and express RA pathway target gene polypeptides exhibiting normal activity may be introduced into cells that do not contain sequences susceptible to whatever antisense, ribozyme, or triple helix treatments are being utilized. Alternatively, it may be preferable to coadminister normal RA pathway target gene protein into the cell or tissue in order to maintain the requisite level of cellular or tissue RA pathway target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for RA pathway target gene protein, and in particular, mutant gene protein, and interfere with its activity may be used to inhibit mutant RA pathway target gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, etc.

In instances where a RA pathway target gene protein is intracellular and whole antibodies are used, internalizing antibodies may be preferred. However, lipofectin liposomes may be used to deliver the antibody or a fragment of the Fab region that binds to the RA pathway target gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the RA pathway target gene protein may be used. Such peptides may be synthesized chemically or produced via recombinant DNA technology using methods well known in the art (see, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular RA pathway target gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

N. Diagnostic Uses

The polynucleotides, polypeptides, variants, targets and antibodies to any one of these molecules can, in addition to previously mentioned therapeutic applications, be used in one or more of the following methods: 1) detection assays (e.g. chromosomal mapping, tissue typing, forensic biology), and 2) predictive medicine (e.g. diagnostic or prognostic assays, pharmacogenomics and monitoring clinical trials). Thus, for example, agents may be used to detect a specific mRNA or gene (e.g. in a biological sample) for a genetic lesion. Similarly, agents described herein may be applied to the field of predictive medicine in which diagnostic assays or prognostic assays, pharmacogenomics, and monitoring clinical trials are used for predictive purposes to thereby treat an individual prophylactically.

Accordingly, one aspect of the present invention relates to diagnostic assays for determining expression of a polypeptide or nucleic acid of the invention and or activity of said agent of the invention, in the context of a biological sample to thereby determine whether an individual is afflicted with a disease or disorder, or is at risk of developing a disorder, associated with aberrant expression or activity of a polypeptide or polynucleotide of the invention.

Alternatively, the invention provides methods for detecting expression of a nucleic acid or polypeptide of the invention or activity of a polypeptide or polynucleotide of the invention in an individual to thereby select appropriate therapeutic or prophylactic agents for that individual (referred to herein as “pharmacogenomics”). Pharmoacogenomics allows for the selection of agents (e.g. drugs) for therapeutic or prophylactic treatment of an individual based on the genotype of the individual (e.g. the genotype of the individual examined to determine the ability of the individual to respond to a particular agent). Still another aspect of the invention pertains to monitoring the influence of agents (e.g. drugs or other compounds) on the expression or activity of a polypeptide or polynucleotide of the invention in clinical trials.

1. Detection Assays

Portions or fragments of the polynucleotide sequences of the invention can be used in numerous ways as polynucleotide reagents. For example, these sequences can be used to i) map their respective genes on a chromosome and, thus, locate gene regions associated with genetic diseases; ii) identify an individual from a minute biological sample (tissue typing); and iii) aid in forensic identification of biological samples.

a. Gene and Chromosome Mapping.

Once the sequence (or portion of a sequence) of a gene has been isolated, this sequence can be used to identify the entire gene, analyze the gene for homology to other sequences (i.e., identify it as a member of a gene family such as EGF receptor family) and then map the location of the gene on a chromosome. Accordingly, nucleic acid molecules described herein or fragments thereof, can be used to map the location of the gene on a chromosome. The mapping of the sequences to chromosomes is an important first step in correlating these sequences with genes associated with disease.

Briefly, genes can be mapped to chromosomes by preparing PCR primers from the sequence of a gene of the invention. These primers can then be used for PCR screening of somatic cell hybrids containing individual chromosomes. Only those hybrids containing the human gene corresponding to the gene sequences will yield an amplified fragment (For review of this technique se D'Eustachio, P. and Ruddle, F. H. (1983) “Somatic cell genetics and gene families.” Science 220:919-924). Alternative methods of mapping a gene to its chromosome include in situ hybridization (see, for example, Fan, Y. S. et al. (1990) “Mapping small DNA sequences by fluorescence in situ hybridization directly on banded metaphase chromosomes.” PNAS 87:6223-27), pre-screening with labeled flow sorted chromosomes (CITE), and pre-selection by hybridization to chromosome specific cDNA libraries. Furthermore, fluorescence in situ hybridization (FISH) of a DNA sequence to a metaphase chromosome spread can further be used to provide a precise chromosomal location in one step (see “Human Chromosomes: A Manual of Basic Techniques”, Pergamon Press, New York, 1988). Lastly, with the completion (in the not-to-distant future) of the sequencing of the human genome, chromosome mapping will very quickly switch from elaborate, hands-on methods of mapping genes, to simple database searches.

Once the sequence (or portion of a sequence) of a gene has been isolated, these agents can be used to assess the intactness or functionality of a particular gene. Comparison of affected and unaffected individuals can begin with looking for structural alterations in the chromosomes such as deletions, inversions, or translocations that are based on that DNA sequence. Once this is accomplished, the physical position of the sequence on the chromosome can be correlated with genetic data map. (such data are found, for example in McKusick, V. “Mendialian Inheritance in Man” available on-line through John Hopkins University Welch Medical Library). The relationship between genes and disease, mapped to the same chromosomal region can then be identified through linkage analysis (co-inheritance of physically adjacent genes), described in e.g. Egeland, J. A. et al. (1987) “Bipolar affective disorders linked to DNA markers on chromosome 11.” Nature, 325:783-787). Alternatively, polynucleotide sequences can be used as probes in Southern Blot analysis to identify alterations in the organization of the gene of interest and surrounding regions. Ultimately, complete sequencing of genes from several individuals can be performed to confirm the presence of a mutation and to distinguish mutations from polymorphisms. If a specific mutation is observed in some or all individuals affected by a particular disease, but not in any unaffected individuals, then the mutation is likely to be the causative agent of the particular disease.

b. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. The sequences of the present invention are useful as additional DNA markers for RFLP mapping (described in U.S. Pat. No. 5,272,057).

Furthermore the sequences of the present invention can be used to determine the actual base-by-base DNA sequence of selected portions of an individual's genome. Thus, the nucleic acid sequences described herein can be used to prepare two PCR primers from the 5′ and 3′ ends of the individual's DNA and subsequently sequence it. Panels of corresponding DNA sequences from individuals, prepared in this manner, can provide unique individual identifications, as each individual will have a unique set of such DNA sequences due to allelic variation. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per 500 bases. Thus, each of the sequences described herein may be, to some degree, used as a standard against which DNA from an individual can be compared for identification purposes.

c. Forensic Biology

In addition the sequences described herein can be used in forensic biology. Forensic biology is a scientific field employing genetic typing of biological evidence found at a crime scene as a means for positively identifying, for example a perpetrator of a crime. To make such an identification, PCR-based technology can be used to amplify DNA sequences taken from very small biological samples such as tissues, (e.g. hair, skin, or body fluids). The amplified sequence can then be compared to a standard thereby allowing identification of the origin of the biological sample.

The sequences of the present invention can be used to provide polynucleotide reagents (e.g. PCR primers) targeted to specific loci in the human genome, which can enhance the reliability of DNA-based forensic identifications by, for example, providing another “identification marker” (i.e. another DNA sequence that is unique to a particular individual. The nucleic acid sequences described herein can further be used to provide polynucleotide reagents e.g. labeled or labelable probes, which can be used in, for example, an in situ hybridization technique, to identify a specific tissue. This technique can be exceedingly useful in cases where a forensic pathologist is presented with a tissue of unknown origin. Panels of such probes can be used to identify tissue by species and/or organ type.

O. Predictive Medicine

Portions or fragments of the polynucleotide sequences of the invention can be used for predictive purposes to thereby treat an individual prophylactically.

1. Diagnostic/Prognostic Assays

One method of detecting the presence or absence of a polypeptide or nucleic acid in a biological sample is to expose that sample to an agent that recognizes the entity in question. A preferred agent for detecting mRNA or genomic DNA is a labeled nucleic acid probe capable of hybridizing to the sequence one is attempting to detect (for instance, the sequence of the invention). The nucleic acid probe can be, for example, a full length cDNA, or a portion thereof such as an oligonucleotide of at least 15, 30, 50, 100, 250, or 500 nucleotides in length and sufficient to specifically hybridize under stringent conditions to a mRNA or genomic DNA encoding the invention. The term “labeled” in this context refers to modifications in said sequences including, but not limited to, biotin labeling that can then be detected with a fluorescently labeled streptavidin, or ³²P labeling.

A preferred agent for detecting a polypeptide of the invention is an antibody or peptide capable of binding to the invention, preferably an antibody with a detectable label. Antibodies can be polyclonal or more preferably, monoclonal. An intact antibody, or a fragment thereof (e.g. a Fab or F(ab)₂) can be used. The term “labeled” in this context refers to direct labeling of the probe or antibody by coupling (i.e. physical linking) a detectable substance to the probe or antibody, such as a fluorescent labeled moiety or biotin.

The detection methods of the invention can be used to detect mRNA, protein, or genomic DNA in a biological sample in vitro as well as in vivo. For example, in vitro techniques for detection of mRNA include (but are not limited to) Northern Blot hybridization and in situ hybridizations. In vitro techniques for detection of a polypeptide of the invention include enzyme linked immunosorbent assays (ELISA's), Western blots, immunoprecipitations, and immunofluorescence.

The invention also encompasses kits for detecting the presence of a polypeptide or nucleic acid of the invention in a biological sample. Such kits can be used to determine if a subject is suffering from or is at increased risk of developing a disorder associate with aberrant expression of a polypeptide or polynucleotide of the invention. For instance, the kit can comprise a labeled compound or agent (as well as all the necessary supplementary agents needed for signal detection e.g. buffers, substrates, etc . . . ) capable of detecting the polypeptide, or mRNA in the sample (e.g. an antibody which binds the polypeptide or a oligonucleotide probe that binds to DNA or mRNA encoding the polypeptide).

The methods of the invention can also be used to detect genetic lesions or mutations in a gene of the invention, thereby determining if a subject with the lesioned gene is at risk for a disorder characterized by aberrant expression or activity of an agent of the invention. In preferred embodiments, the methods include detecting the presence or absence of a genetic lesion or mutation characterized by at least one alteration affecting the integrity of the agent of the invention. For example, such genetic lesions or mutations can be detected by ascertaining the existence of at least one of: 1) a deletion of one or more nucleotides from a gene; 2) an addition of one or more nucleotides to a gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA; 8) a non-wild type level of the protein encoded by the gene; 9) an allelic loss of the gene; and 10) an inappropriate post translational modification of the protein encoded by the gene. Many techniques can be used to detect lesions such as those described above. For instance, mutations in a selected gene from a sample can be identified by alterations in restriction enzyme cleavage patterns. In this procedure, sample and control DNA is isolated, digested with one or more restriction endonucleases, and fragment length sizes (determined by gel electrophoresis) are compared. Observable differences in fragment length sizes between sample and control DNA indicates mutations in the sample DNA. Additional techniques that can be applied to detecting mutations include, but are not limited to, detection based on direct sequencing, PCR-based detection of deletions, inversions, or translocations, detection based on mismatch cleavage reactions (Myers, R. M. et al. (1985) “Detection of single base substitutions by ribonuclease cleavage at mismatches in RNA:DNA duplexes.” Science 230:1242), and detection based on altered electrophoretic mobility (e.g. SSCP, see, for example, Orita, M. et al. (1989) “Detection of polymorphisms of human DNA by gel electrophoresis as single-strand conformation polymorphisms.” PNAS 86:2766).

2. Pharmacogenetics

Pharmacogenetics deals with clinically significant hereditary variation in the response to drugs due to altered drug disposition and altered action in affected persons (see Linder, M. W. et al. (1997) “Pharmacogenetics: a laboratory tool for optimizing therapeutic efficiency.” Clin Chem. 43(2):254-266). In general, two types of pharmacogenetic conditions can be differentiated. There are genetic conditions transmitted as a single factor altering the way drugs act on the body, referred to as “altered drug action”. Alternatively, there are genetic conditions transmitted as single factors altering the way the body acts on drugs (referred to as “altered drug metabolism”). These two conditions can occur either as rare defects, or as polymorphisms. For example, glucose-6-phosphate dehydrogenase deficiency is a common inherited enyzmopathy in which the main clinical complication is haemolysis after ingestion of oxidant drugs (e.g. anti-malarials, sulfonamides etc.).

The activity of drug metabolizing enzymes is a major determinant of both the intensity and duration of drug action. The discovery of genetic polymorphisms of drug metabolizing enzymes (e.g. N-acetyltransferase 2 (NAT2) and cytochrome P450 enzymes (CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM which all lead to the absence of functional CYP2D6. Poor metabolizers of this sort quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. At the other extreme are the so-called ultra rapid metabolizer who do not respond to standard doses. Recently, the molecular basis of ultra rapid metabolism has been identified to be due to CYP2D6 gene amplification.

Thus the in the context of pharmacogenetics, an agent of the invention can be used to determine or select appropriate agents for therapeutic prophylactic treatment of the individual. In addition, pharmacogenetic studies can be used to apply genotyping of polymorphic alleles encoding drug-metabolizing enzymes to the identification of an individuals drug responsiveness phenotype.

3. Monitoring of Effects During Clinical Trials

Monitoring the influence of agents that effect the expression or activity of a polypeptide or polynucleotide of the invention can be applied in clinical trials. For example, the effectiveness of a drug directed toward a target identified by the invention and intended to treat a particular ailment, can be monitored in clinical trials of subjects exhibiting said ailment by monitoring the level of gene expression of the target, activity of the target, or levels of the target of the invention. Thus in a preferred embodiment, the present invention provides a method for monitoring the effectiveness of treatment of a subject with an agent by comprising the steps of (i) obtaining a pre-administration sample from a subject prior to administration of the agent; (ii) detecting the level of the polypeptide or polynucleotide of the invention in the pre-administration sample; (iii) obtaining one or more post-administration samples from the subject; (iv) detecting the level or activity of said target of the invention in the post-administration samples, (v) comparing the level of said target of the invention in the post administration sample with levels in the pre-administration samples, and (vi) altering the administration of the agent to the subject accordingly.

### EXAMPLES

The following examples are intended to further illustrate certain preferred embodiments of the invention, and are not limiting in nature.

### Example 1

Construction of a Retinoic Acid Sensitive Reporter Cell Line

A. Construction of the RARE-GFP Reporter

The pRETRO-ON retroviral vector (Clontech, #6158) was digested with EcoRI and XhoI to remove the reverse tetracyclin-controlled transactivator (rtTA) and SV40 promoter sequences from the vector backbone. Subsequently, the mini-CMV and adjoining EGFP coding sequence were PCR amplified from an in-house plasmid vector (pVT304-2, original source, Clontech, Genbank Acc. N. U55763) using oligonucleotides oVT 818 and oVT 819 that had XhoI and EcoRI restriction sites engineered into the primers (oVT 818:5′ CGTACGGGAATTCGTCGACCGGTCAT GGCTG; oVT 819:5′ CGTTACGGCTCGAGGTAGGCGTGTACGGTGGG). The mini-CMV-EGFP fragment was then digested with EcoRI and XhoI and inserted into the modified pRETRO-ON backbone. This mosaic construct was then digested with EcoRI, blunted with Klenow, and religated to eliminate the EcoRI site. A similar procedure was performed to knock out the remaining NotI site. Subsequently, a multiple cloning site (MCS) containing EcoRI, NotI, and XhoI sites was inserted upstream of the mini-CMV. This was accomplished by annealing together two complementary oligonucleotides (oVT 823:5′ TCGAGACAGAATTCGCGGCCGCA, and oVT 824:5′ TCGATGCGGC CGCGAATTCTGTC) that contained the appropriate restriction sites flanked by one intact and one crippled XhoI site. The final double stranded annealing product was ligated into the XhoI site of our vector and sequenced to identify a clone with the correct orientation.

An 800-base pair RARE promoter sequence from the RAR β gene (de Thé, H. et al. (1990) “Identification of a retinoic acid responsive element in the retinoic acid receptor beta gene.” Nature 343:177-180) was selected as the cis-regulatory element for this experiment. Using standard methods familiar to those of ordinary skill in the art, the RARE was PCR amplified from genomic DNA taken from HL60 cells grown in tissue culture (oVT 825-5′ AGAACACACAGC TGGTAAGTGGCAGACCTGG 3′ and oVT 827-5′ ACGCTCACTTGA AAGCCACTTGGGATGGGCCC 3′). The RARE was then cloned in the correct orientation upstream of the EGFP-encoding sequence using the XhoI/EcoRI sites that was engineered into the PCR primers. This placed the RARE directly upstream of a minimal CMV promoter, thereby bringing the EGFP reporter under the control of the endogenous RA-inducible cellular pathway. The hybrid pRETRO-On, RARE controlled EGFP construct (designated pVT355) was then co-transfected with VSV-G envelope expression plasmid into 293 gp packaging cells (gift of I. Verma, Salk Institute) using LipofectAmine (Life Technologies). In this technique, 1, 3>10⁶ cells of the packaging cell line (293 gp) are seeded into a T175 flask. On the second day, two tubes, one carrying 15 ug of library DNA+10 ug of envelope plasmid (pCMV-VSV.G-bpa)+1.5 ml DMEM (serum free), the second carrying 100 ul of LipofectAMINE (Gibco BRL)+1.5 ml DMEM (serum free) are mixed and left at room temperature for 30 minutes. Subsequently, the two tubes are mixed together along with 17 ml of serum free DMEM. This cocktail is referred to as the “transfection mix.” Previously plated 293 gp cells are then gently washed with serum free media and exposed to 20 ml of the transfection mix for 4 hours at 37° C. Following this period, the transfection mix can be removed and the cells are incubated with complete DMEM (10% serum) for a period of 72 hours at 37° C. On Day 4 or 5, the media (now referred to as “viral supernatant”) overlying the 293 gp cells is collected, filtered through a 0.45μ filter and frozen down in at −80° C. Alternatively, retroviral DNA can be packaged using a technique that is referred to herein as the “CaCl₂ Method”. In this method, 5×10⁶ cells of the packaging cell line (293 gp) are seeded into a 15 cm² flask on Day 1. On the following day, the media is replaced with 22.5 mls of modified DMEM. Subsequently, a single tube carrying 22.5 μg of retroviral library DNA and 22.5 μg of envelope expression plasmid (pCMV-VSV.G-bpa) is brought to 400 μl with dH₂O, to which is added 100 μl of CaCl₂ (2.5M) and 500 μl of BBS (drop-wise addition, 2×solution=50 mM, BES (N,N-bis(2-hydroxyethyl)-2-aminoethane-sulfonic-acid), 280 mM NaCl, 1.5 mM Na₂HPO₄, pH 6.95). After allowing this retroviral mixture to sit at room temperature for 5-10 minutes, i.e. is added to the 293 gp cells in a drop-wise fashion, and the cells are then incubated at 37° C. (3% CO₂) for 16-24 hours. The media is then replaced and the cells are allowed to incubate for an additional 48-72 hours at 37° C. At that time, the media containing the viral particles is then collected, filtered through a 0.45μ filter and frozen down at −80° C. Retroviral supernatant can subsequently be thawed and used directly to infect WM35 melanoma cells (a gift of M. Herlyn, Wistar Institute).

B. Identification of an ATRA-Sensitive Cell Line

Several melanoma cell types were screened for responsiveness to RA. A host population of RA-responsive melanoma cells was identified as follows. When the WM35 cell line was exposed to 5 μM RA (Sigma), they exhibited an increase in granularity. The visual observation of RA-induced morphological changes were consistent with FACS analysis that showed RA-induced changes in the Forward vs. Side Scatter Plot of WM35 cells. Following standard procedures common to the art, a population of 1×10⁷ WM35 cells were infected with the pVT355 retroviral supernatant for 24 hours, using a 20% vol/vol of retroviral supernatant to complete media (KBM catalogue no.CC3101, Clonetics) plus 2% FBS. The cells were then allowed to recover for 24 hours in complete media. Cells containing stable inserts of the pVT355 vector were selected by culturing cells in 1 μg/ml puromycin (Sigma).

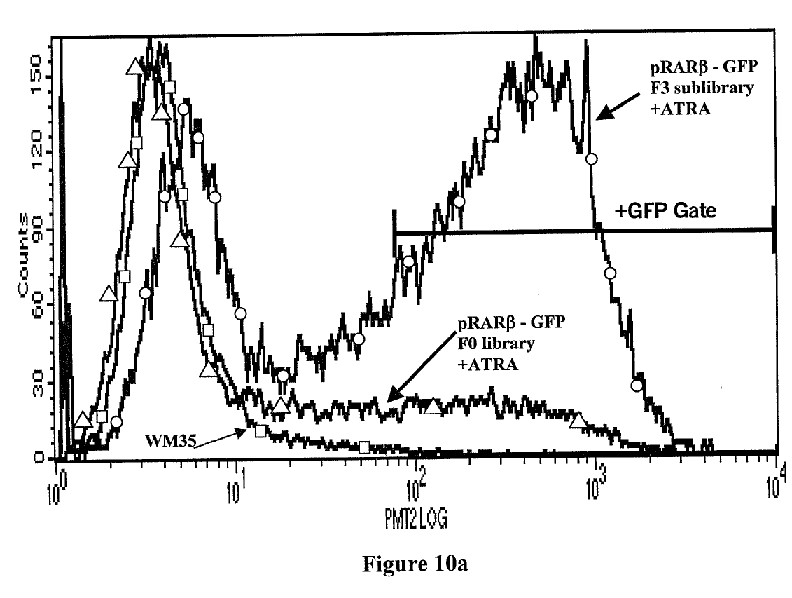

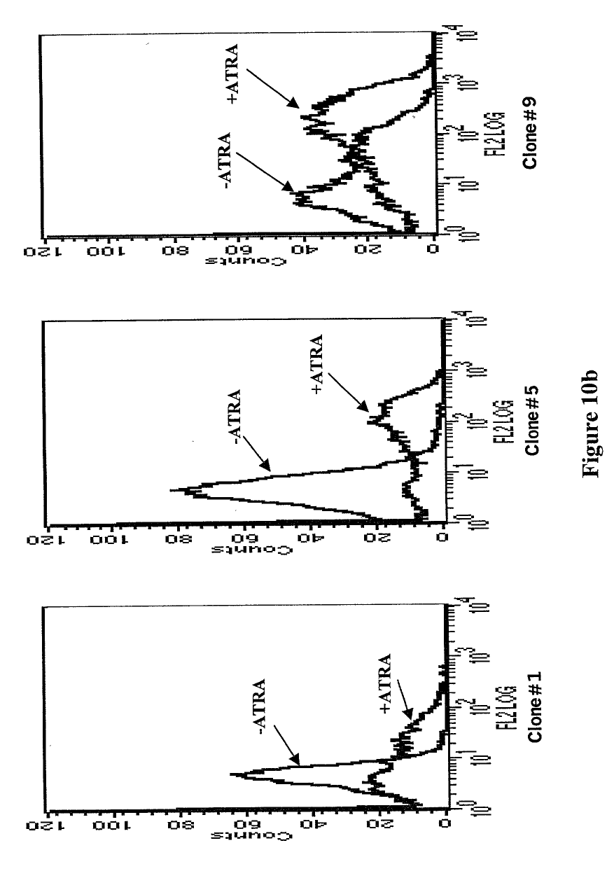

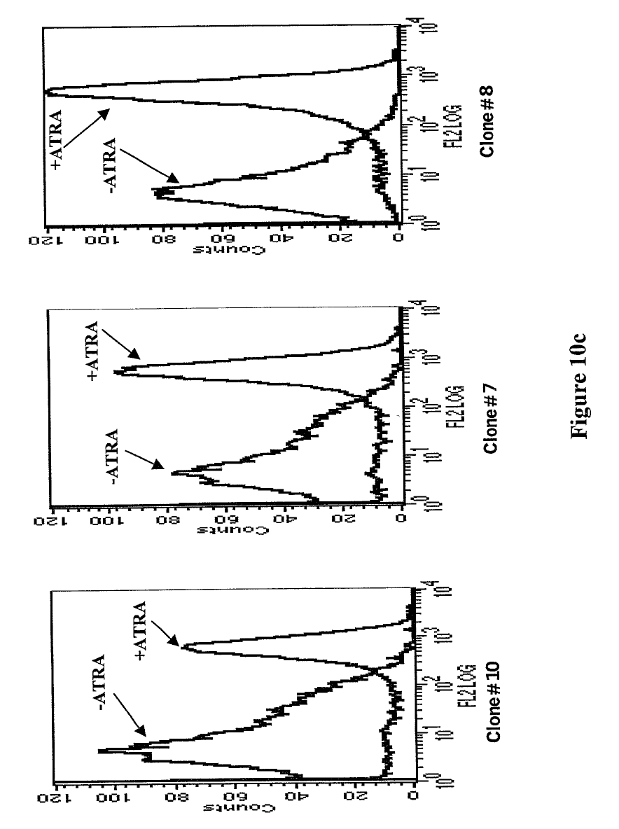

The puromycin resistant cell population was transferred to media supplemented with 2% charcoal-stripped FBS serum (CBI, Cocalico Biologicals Inc.) to reduce or eliminate retinoids present in FBS. To isolate ATRA responsive clones, cells were grown in 2% CBI media for three days followed by addition of 5 μM all-trans retinoic acid dissolved in 100% ethanol (ATRA, Sigma). After 48 hrs of induction with ATRA, the EGFP-expressing cells (referred to as “F1”) were recovered using a fluorescent activated cell sorter (FACS, Coulter EPICS Elite). The EGFP⁻ gate (<50 fluorescent units) and the EGFP⁺ gate (>50 fluorescent units) were selected based on the autofluorescent level of WM35 cells and the expression level of a hCMV-EGFP in WM35 cells (a cell line that constitutively expresses EGFP), respectively. Sorted cells were propagated for 10-14 days and the cycle of CBI-replacement followed by ATRA treatment was repeated for two additional rounds to enrich for ATRA responsive clones (FIG. 10).

To obtain a robust RA-responsive clone, the following procedure was performed: some 2000 RA-responsive cells obtained from the above FACS cycling procedure were plated on 150 mm plates to allow clonal isolation. After 7 days there were approximately 100 cells/colony at which time the complete media (+FBS) was replaced with CBI supplemented media. After 3 days in CBI media, 5 μM RA was added to the plate. Two days after induction of the RARE-EGFP reporter with RA, 24 independent colonies that contained a very high percentage of EGFP expressing cells were picked for further analysis. One clone—designated Clone 8—showed minimal expression of EGFP in CBI and a 290-fold induction when treated with RA. Addition of as little as 100 nM RA caused induction of EGFP in this clonal population of cells, and shifted the vast majority of cells (>95%) into the “bright” FACS gate. As FACS analysis revealed minimal overlap in the fluorescence signals of the RA⁺ and RA⁻ populations (FIG. 10), Clone 8 was selected as the reporter line for further studies.

C. Testing the Sensitivity of Clone 8 to Synthetic Perturbagens.

In order to test the responsiveness of the RA reporter cell line, a population of Clone 8 cells was subjected to a synthetic perturbagen to verify that (1) the fluorescence induction was in fact related to RA, and (2) the reporter construct was sensitive to the presence of the synthetic perturbagen. This was accomplished by introducing into the Clone 8 cells a known RA pathway inhibitor—a synthetic, dominant-negative perturbagen designated RARΔ403—as follows.

A truncated RARαΔ403 construct encoding a sequence containing the DNA binding domain and an RXR binding domain, but lacking 59 amino acids from the C-terminus, (portions of the RA ligand binding and transcriptional activation domain, AF-2) was subcloned via reported methods (see, Tsai, S. et al. (1992) “A mutated retinoic acid receptor-alpha exhibiting dominant-negative activity alters the lineage development of a multipotent hematopoietic cell line.” Genes and Development, 6:2258-2269). Specifically, the truncated RARα cDNA was PCR amplified from brain human brain cDNA and inserted into the pBABE retroviral vector (NeO^{R}).This synthetic perturbagen, designated Δ403, is believed to be capable of forming heterodimers with the RXR subunit and binding the RARE promoter sequences, yet is unresponsive to the addition of exogenous RA. As a result, Δ403-bearing host cells should remain “dim” in the presence of RA. To test the ability of Δ403 to alter Clone 8 fluorescent properties, the dominant negative inhibitor of the RA pathway was introduced into the Clone 8 cells in three separate modes: (i) as a naked cDNA, (ii) inserted into a non-fluorescent EGFP scaffold, “dGFP,” which bears a Tyr→Phe mutation at amino acid residue 66, and (iii) inserted onto the 3′ end of the glutathione S-transferase “GST” scaffold, commonly used for purification of fusion proteins. The various Δ403 constructs were packaged in 293 gp's as described previously, and infected into a population of Clone 8 cells as described elsewhere herein.

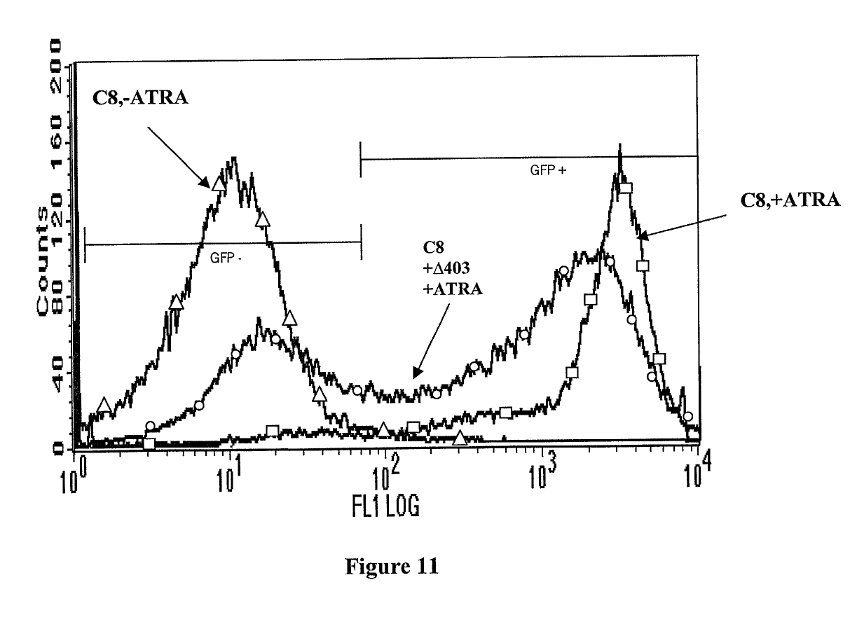

The RA responsiveness of the various Δ403-bearing cell populations was compared to the basal (untreated) Clone 8 population as follows. Each cell population was cultured in the presence of 100 nM RA, and subjected to FACS analysis. Approximately 98% of the control Clone 8 population shifted to the “bright” gate in response to the RA, while approximately 2% remained non-responsive to RA. In contrast, a significant percentage of the cells in which the naked Δ403 construct had been introduced into the Clone 8 background remained in the “dim” gate in the presence of RA. Individual clones derived from this population displayed a penetrance ranging from approximately 10-93%, with the Δ403 population as a whole showing roughly 34% penetrance. The populations with the scaffolded Δ403 inserts showed a similar penetrance when fused to the dGFP and GST proteins (38% and 41% in the “dim gate, respectively). The attenuated response of Clone 8 to RA with the synthetic inhibitory perturbagen, Δ403, provides support that induction of EGFP is due to RA, and indicates that the Clone 8 reporter cell line was sensitive to the presence of perturbagens. (FIG. 11).

To measure the responsiveness of C8 to perturbagens that activate the pathway, a synthetic perturbagen, RAR-VP16, is constructed and introduced into the C8 cell line. (Underhill, T. M. et al. (1994) “Constitutively active retinoid receptors exhibit interfamily and intrafamily promoter specificity.” Mol Endocrinol 8(3):274-85). Cells containing the RAR-VP16 activator are then grown in CBI supplemented media lacking ATRA and examined by FACS to assess the effects of the chimeric molecule on the distribution of cells in the bright and dim gates.

### Example 2

Preparation and Transfer of a cDNA Library

Using techniques that are familiar to individuals in the art, randomly primed cDNA libraries were used as a source of sequences encoding putative RA-pathway activating and blocking agents. As one non-limiting example of how to construct such a library, polyA mRNA derived from placental tissue was PCR amplified using a random 9-mer linked to a unique SfiI sequence (“SfiA”), followed by an additional set of nucleotides that is used later for library amplification (OVT 906:5′ ACTCTGGACTAG GCAGGTTCAGTGGCCATTATGGCC(N)₉). The product of this reaction was size selected (>400 base pairs) and subjected to RNAse A/H treatment to remove the original RNA template. The remaining single stranded DNA was then subjected to a second round of PCR using a random hexamer nucleotide sequence linked to a second unique SfiI sequence (“SfiB”) which was again followed by an additional set of nucleotides for future library amplification: (OVT 908:5′ AAGCAGTGGTGTCAACG CAGTGAGGCCGAGGCGGCC (N)₆). The final product of this reaction, a double stranded cDNA, was blunted/filled with Klenow Fragment (New England BioLabs), size selected, PCR amplified (OVT 909:5′ ACTCTGGACTAGGCAGGTTCAGT and OVT 910:5′ AAGCAGTGGTGTCAACGCAGTGA), digested with SfiI (New England BioLabs), and inserted into a retroviral vector (pVT 352.1, pBabe). As a result of these procedures, the sequences encoding the perturbagens were inserted at the 3′ end of the non-fluorescent variant of EGFP (dEGFP). Expression of the dEGFP-perturbagen fusion gene (as well as the neomycin resistance gene present in the retroviral vector) was driven by the 5′ LTR of pBabe. The library (˜12×10⁶ in size) was then packaged in 293 gp cells (laboratory of I. Verma) and retroviral supernatant was generated. Subsequently, 50 million Clone 8 cells were infected with the viral supernatant. Approximately half of the starting cells (25 million) were found to contain virus after a ten-day selection in neomycin sulfate (550 ug/ml, Life Sciences Technology). This population was expanded to 50 million and used in subsequent Trans-FACS phenotypic assay selection to identify perturbagens that either 1) inhibit or 2) induced the RA pathway.

### Example 3

Isolation of Perturbagens that Block the RA Pathway

To identify and isolate perturbagens that interfered with the RA pathway, the library containing C8 cells (F0) were grown for 48-72 hours in the presence of ATRA (2% FBS). Subsequently, non-fluorescent, or “dim” cells were collected by FACS. The size and position of the “dim” sort gate was determined from previous work with Clone 8 cells carrying the dominant-negative control perturbagen, RARΔ403. The collected dim cells (referred to as “F1”) were then allowed to expand 10-fold before being re-sorted using the same “dim” gates described previously. Approximately 10% (122,000 cells) of the F1 population fell into the “dim” gate. When the cycling process was repeated on these cells (F2), 25% of the population fell into the “dim” gate. These cells (F3) were subsequently collected and expanded for further investigation. As an alternative procedure, the perturbagen inserts contained in the dim population can be PCR amplified from genomic DNA derived from F1, F2, or F3 populations. This material can subsequently be used to construct new sublibraries that, in turn, can be re-screened for further enrichment.

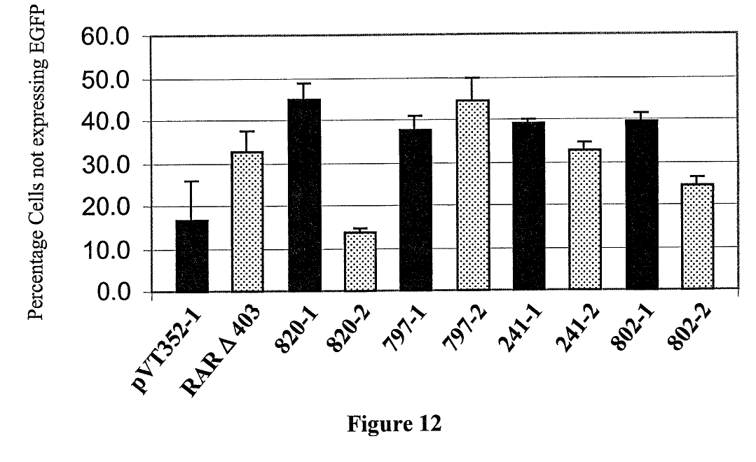

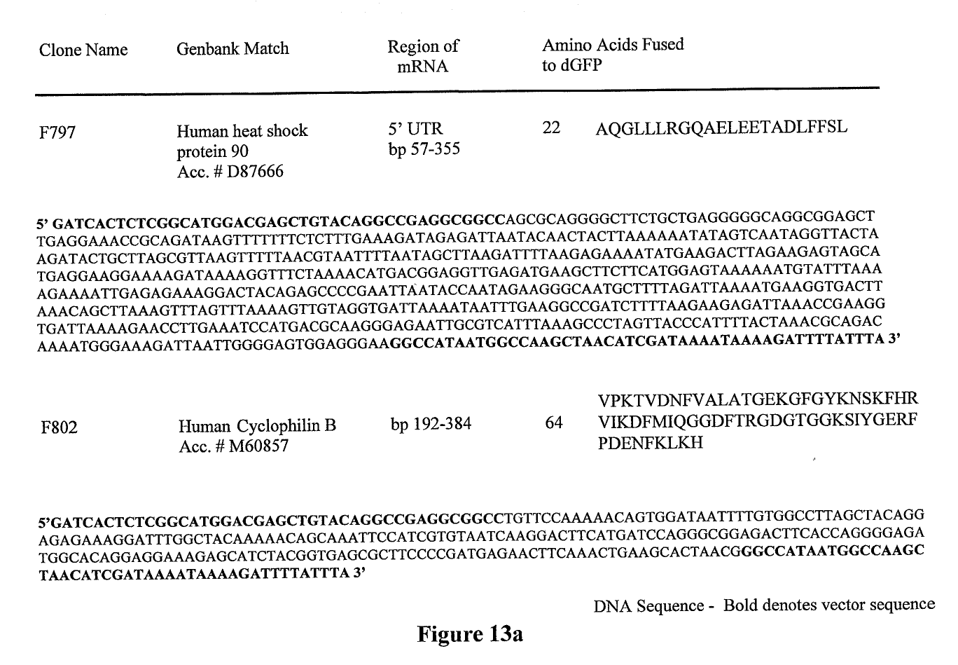

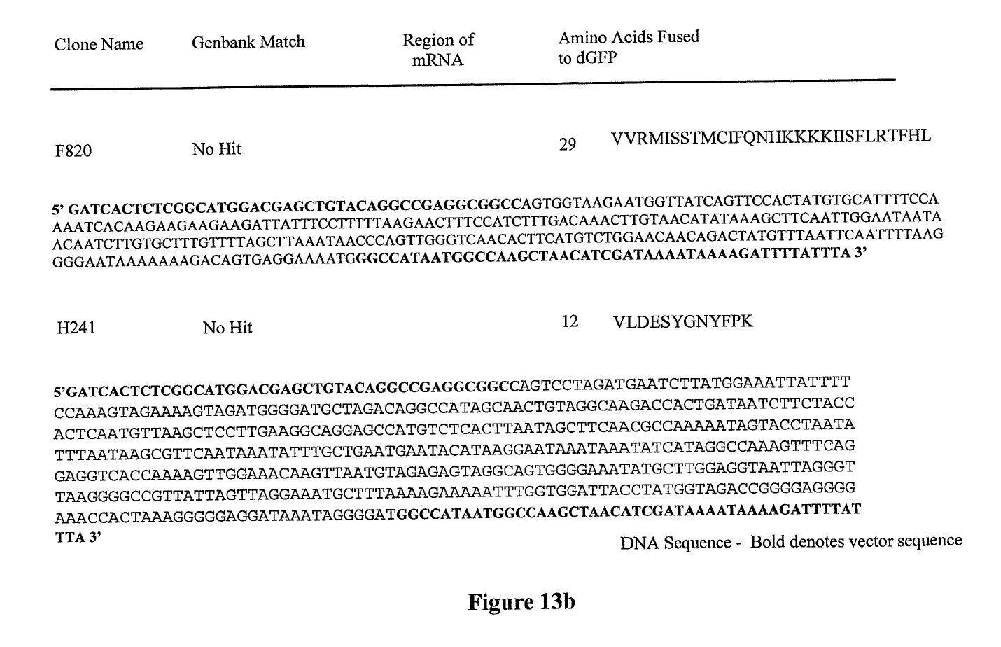

Two thousand cells from the F3 population were plated out and allowed to grow into colonies. These cells were then examined by fluorescent microscopy and forty colonies that exhibited a microscopic phenotype similar to the C8/Δ403 clones (i.e. the colonies appeared to be “speckled” or partially penetrant) were isolated and expanded to numbers sufficient for genomic DNA isolation. The DNA encoding ten of these perturbagens was then PCR amplified using oligonucleotide sequences that flanked the cDNA insert (oVT 181:5′ GGATCACTCTCGGCATGGACGAG and oVT 178:5′ ATTTTATCGATGTTAGCTTGGCCATT), recloned into the original vector (in both the original reading frame and a second reading frame), and tested for their ability to suppress the ATRA induced signal. Of the ten putative perturbagens examined, three (P820, P802, and P797) demonstrated perturbagen activity when retested (FIG. 12). A fourth perturbagen (P241) was isolated after a sublibrary was generated from the F3 population of dim cells. The penetrance of these four clones varied between roughly 20 and 30% and two of the four clones (P797 and P241) exhibited their phenotype in multiple reading frames suggesting the perturbagen was acting at an RNA level rather than as a peptide. Sequence analysis of the proteinaceous perturbagens showed them to vary in length from 30 to 63 amino acids (FIG. 13 a,b). One clone (F802) was identified to be a fragment of cyclophilin. The sequence of the F802 perturbagen is homologous with the internal region of cyclophilin B and encodes the catalytic domain for the protein's peptidyl-prolyl-isomerase activity. F802 is particular intriguing because cyclophilins have been implicated in the folding/stabilization and/or transport of Type I nuclear receptors (e.g. Estrogen receptors).

### Example 4

Isolation of Perturbagens that Activate the RA Pathway

To identify perturbagens that activate the RARE driven reporter, 70 million Clone 8 cells containing the random-primed cDNA library described previously were grown in media containing 2% CBI (charcoal stripped) serum. After five days of growth under these conditions, “bright” cells were sorted from the population, expanded 10×, and then used to prepare a new sublibrary. To create a perturbagen sublibrary, sorted cells were harvested and then used to prepare genomic DNA (Qiagen genomic DNA isolation kit). The DNA encoding the perturbagens was then recovered by PCR amplification using two oligonucleotides that contained homology with sequences flanking the cDNA insertion site (oVT 181:5′ GGATCACTCTCGGCATGGACGAG and oVT 178:5′ ATTTT ATCGATGTTAGCTTGGCCATT). The PCR product was then digested with SfiI, directionally cloned back into the original vector (pVT352.1), and reinfected into fresh Clone 8 cells for a second round of selection. This process was repeated through six consecutive rounds of selection and each new sublibrary was compared with parallel control infections using pVT352.1 that lacked an insert. Following these procedures, individual clones were sequenced and retested.

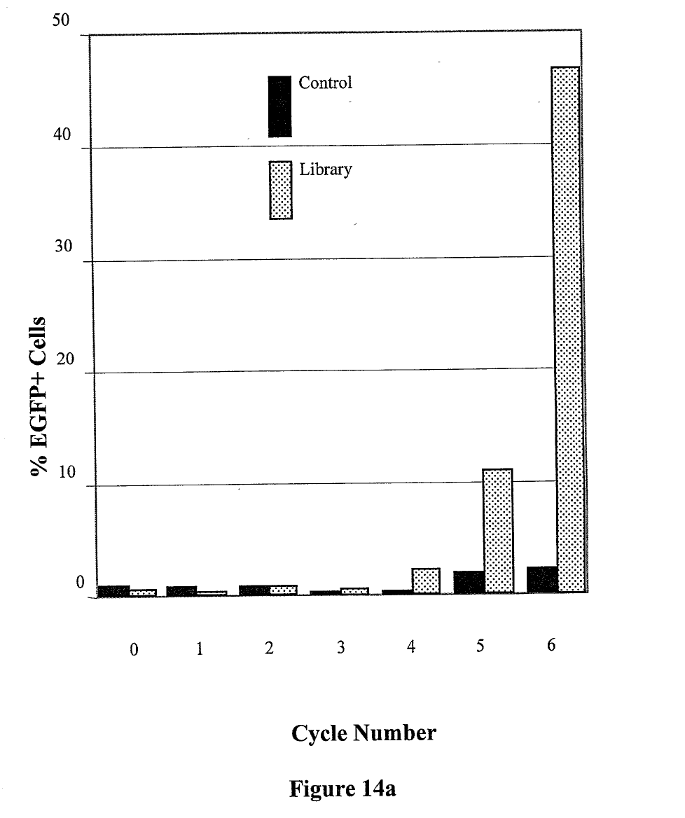

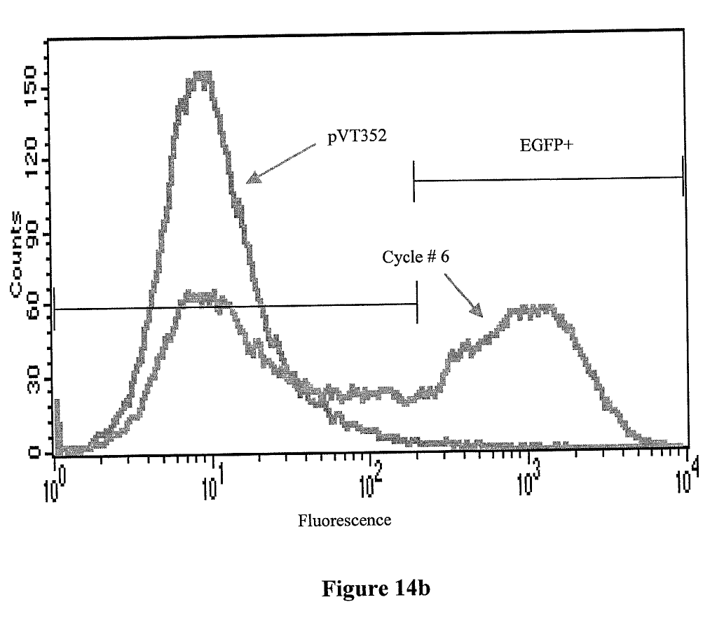

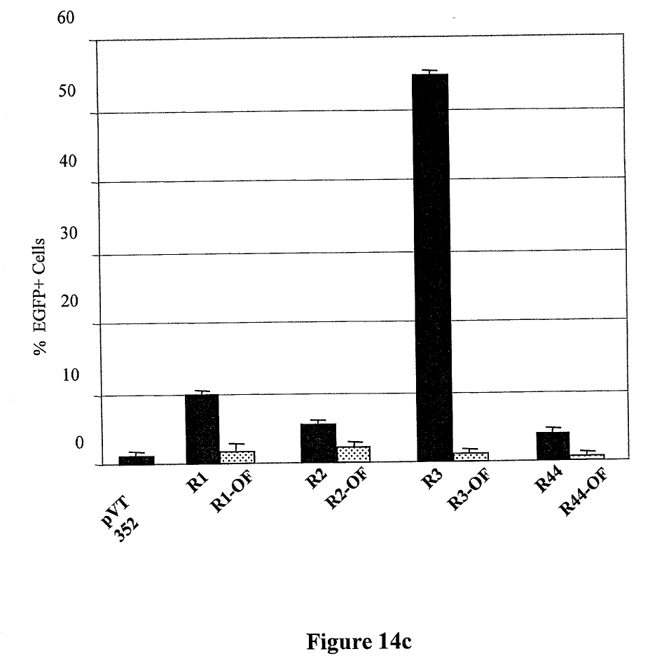

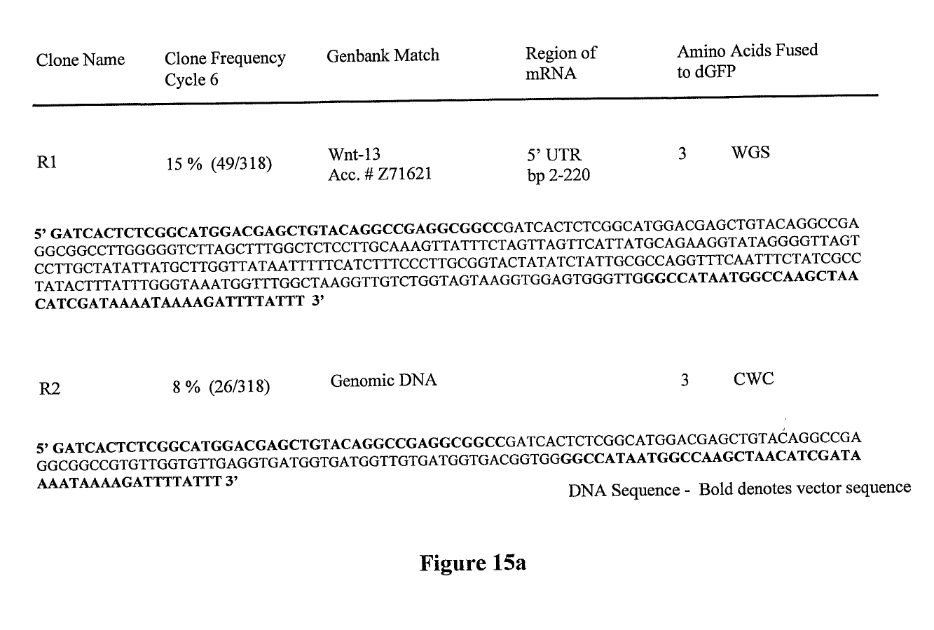

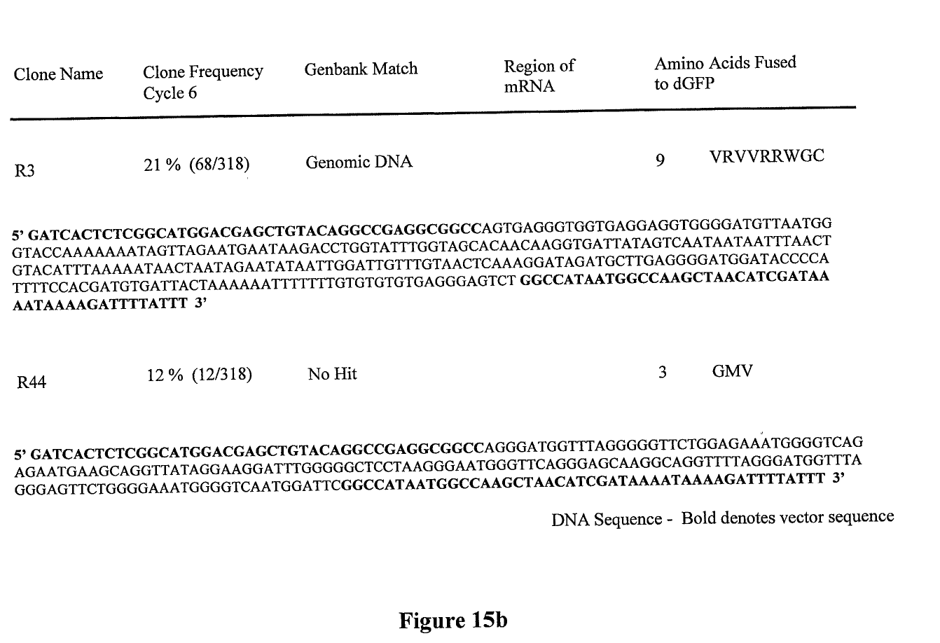

Under the conditions described above, the number of cells falling into the “bright”(induced) gate in the control experiment(s) was consistently below 2.5%. In contrast, the percentage of bright cells in the library-containing samples steadily increased over the course of the final three selections. By the end of six sorts, 47% of the population was found to be “bright” (FIG. 14 a,b). From 318 F6 clones that were sequenced, 13 were chosen to be retested on the basis of the frequency at which they were observed in the F6 population. These perturbagen-encoding sequences were cloned into the original vector (pVT352.1) and a second vector, pVT352.2 that placed the perturbagens in an alternate reading frame. The results of these studies show that while some of these clones exhibited no phenotype, others were capable of inducing RARE reporter expression with variable penetrance (5˜60%, see FIG. 14c). Of the clones that exhibited perturbagen activity, the peptide length varied between 3 and 9 amino acids in length with the largest perturbagen, F6.R3 (9 amino acids), exhibiting the highest penetrance (59% penetrance). In addition, at least two of the perturbagens isolated (F6.R3 and F6.R1) exhibited sequence homology (WGS vs. WGC motif, see FIG. 15).

### Example 5

Isolation of Perturbagen Targets

The following example demonstrates how the targets of perturbagen R3 can be isolated.

A. Yeast Two Hybrid and Immunoprecipitation

The polynucleotide sequence encoding perturbagen R3 was cloned into pVT 2527 using Gap-repair. As a result of these procedures, the 9 amino acids of R3 were fused in-frame with the C-terminus of dGFP, which was, in turn, fused to the LexA binding domain. Using conventional means the vector was then introduced into the yeast strain yVT 87 and mated to a population of yVT 99 cells that contain an episomal, human cDNA library (Proquest Fetal Brain library, Gibco) fused to the C-terminus of the LexA activation domain and regulated by the Gal4 promoter. In addition, the yVT99 strain contains two chromosomal-linked reporter constructs (URA3 AND LEU1 reporter genes) that are operably associated with eight tandem copies of the LexA operator sequence. Diploids containing a copy of both the R3 encoding vector and a member of the cDNA library were then selected by plating the cells on SD−Trp, −His media.

To identify cellular targets of the R3 perturbagen, diploid cells were collected and plated on SD −His, −Trp, −Ura, and −Leu, −Dex, +Gal plates. Cells that were capable of growing and forming colonies under these conditions were then picked and the associated cDNA target was sequenced using standard techniques. Target sequences were then used to search the NCBI BLAST database to determine target identity.

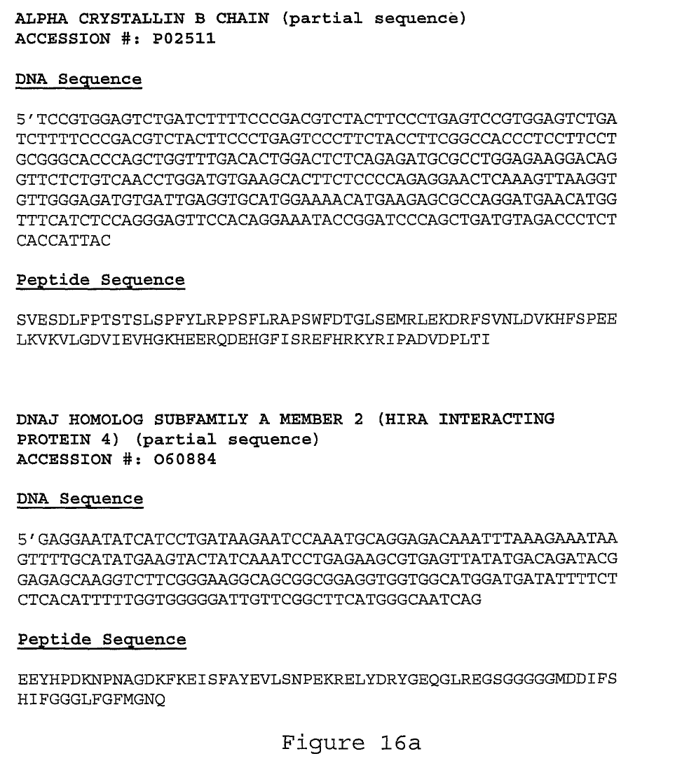

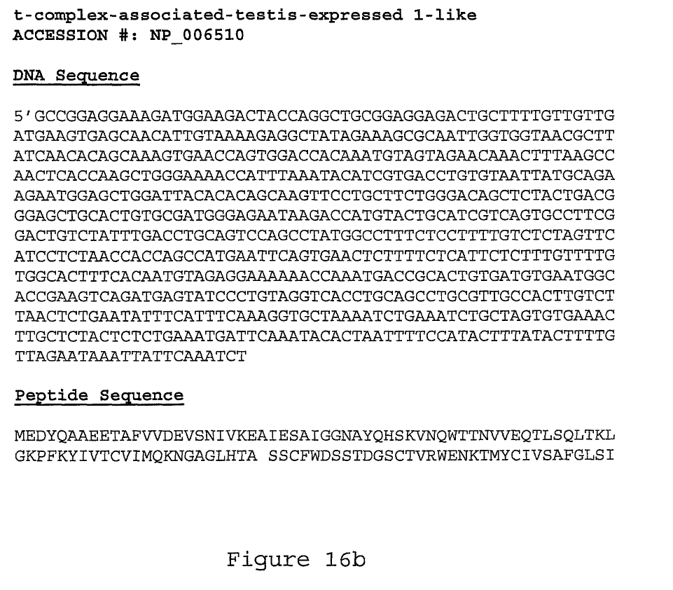

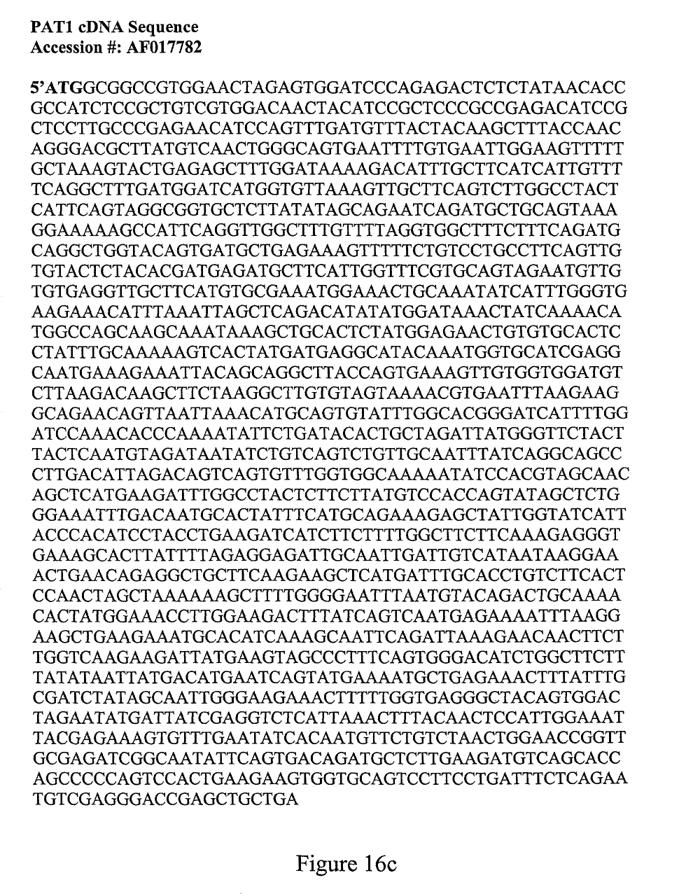

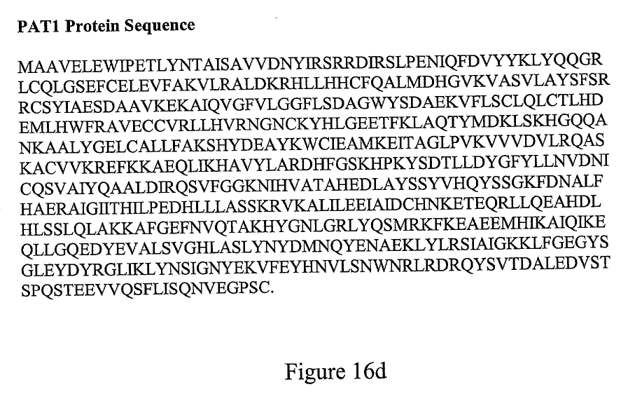

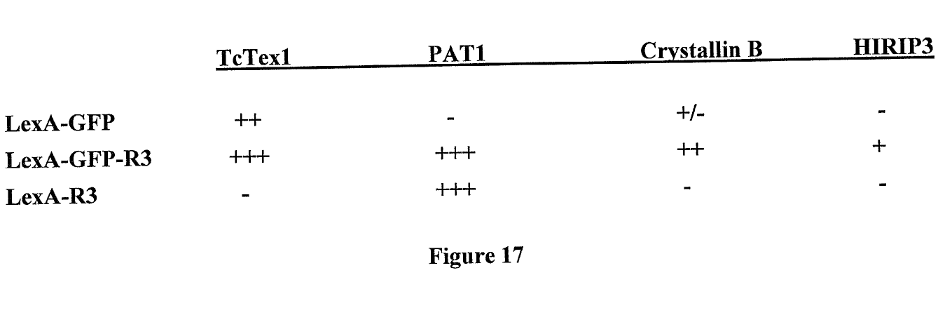

Four potential R3 targets, including PAT1 (a kinesin light chain-related protein accession), TCTEL1, alpha crystallin β chain, and Hira Interacting Protein, were identified by two-hybrid analysis (see FIG. 16 a-d). When each of these cDNAs was cloned into a second vector (pVT 2517) that fused the potential target directly to the LexA BD (i.e. lacking the GFP scaffold) three of the targets (TCTEL1, alpha crystallin β chain, and Hira Interacting Protein) showed a reduction in the strength of the R3-target interaction, suggesting a contribution of the GFP scaffold in these R3-target interactions. FIG. 17. In contrast, the interaction between R3 and the final target, PAT1, proved to be scaffold independent. A search of the PubMed database reported no previously observed interaction between PAT1 and the retinoic acid pathway.

Two procedures were performed to verify the interaction between R3 and the PAT1 gene. First, two independent DNA clones of PAT1 were transformed back into yVT99 and mated with the yVT87 line expressing the R3 perturbagen. When the diploid cells were plated on media that required an interaction between the perturbagen and target for growth, the majority of the cells were observed to proliferate, thus confirming the R3-PAT1 interaction.

In a second procedure, immunoprecipitation (IP) was used to test the R3-PAT1 interaction. Specifically, PAT1 and R3 expression constructs were transiently co-transfected into HEK293 cells by Lipofectamine. Two days post-transfection, 10⁶ cells were rinsed in PBS and lysed in IP buffer: 25 mM Hepes (pH 7.5), 5 mM EDTA, 5 mM EGTA, 50 mM NaCl, 50 mM NaF, 10% (vol/vol) Glycerol, 1% (vol/vol) Triton X-100, 2 mM sodium orthovanadate, 1 mM PMSF, and protease inhibitor cocktail (Sigma Aldrich). Following centrifugation (13K for 10 min at 4° C.), the lysate was cleared by adding 1 ug mouse IgG antibody plus 20 μl Protein A/G plus agarose (Amersham Pharmacia Biotech) at 4° C., 1 hr. The sample was then centrifuged (2500 RPM for 5 min at 4° C.) and the supernatant was treated with 1 μg of anti-GFP monoclonal antibody (e.g. Mouse monoclonal IgG1ₖ Clone 7.1 and 13.1, Roche, 4° C. on rotisserie for 2 hrs) to bring down GFP-R3 and any interacting protein. Subsequent addition and incubation of the sample with 20 μl of Protein A/G plus sepharose beads (4° C. on rotisserie for 2 hrs) allowed isolation of the antibody-GFP-perturbagen-target complex by centrifugation (2500 RPM for 5 min at 4° C.). The resulting pellet was then washed/centrifuged three times in IP-Wash Buffer (IP-buffer/PMSF/Protease inhibitors with 150 mM NaCl, 300 mM NaCl, or 450 mM NaCl) to remove non-specific/low-affinity binding contaminants. Following the final wash the pellet was resuspend in 20 μl 2× sample loading buffer, boiled for 3-5 min, and spun in a microcentrifuge to separate the pellet from the supernatant. The supernatant containing both the perturbagen and the target was then loaded on a SDS-polyacrylamide gel (Novex) and visualized by silver stain (Invitrogen) and/or Western Blot (PVDF membrane) using anti PAT1 antibodies.

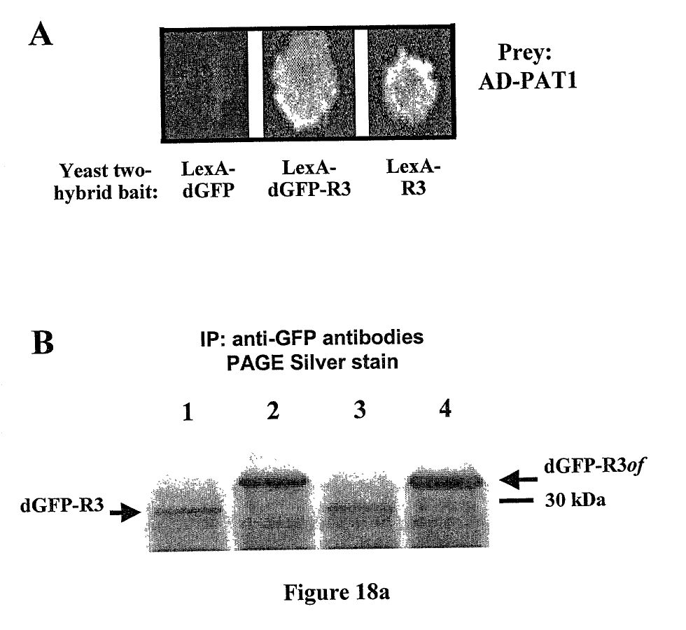

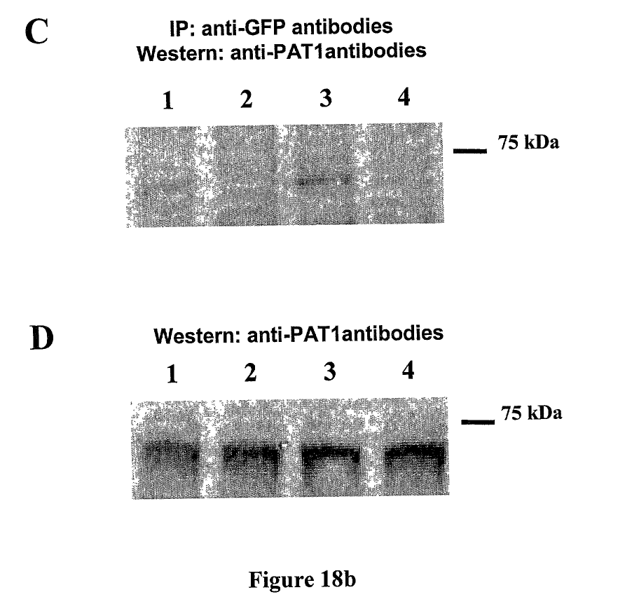

The results of these studies showed that the R3 perturbagen exhibited a strong affinity for PAT1 protein in HEK293 cells. As shown in FIG. 18, Western Blots probed with an anti-PAT1 antibody show a distinctive band that corresponded with the molecular weight of PAT1. In contrast, cells that contained an out-of-frame R3 insert show no equivalent staining pattern, thus supporting the specificity of the R3-PAT1 interaction.

B. Gene Expression Profiling

A second approach, expression profiling, was used to identify R3 target genes. To accomplish this, poly(A)+RNA was isolated from ˜50×10⁶ cells according to the protocols of Qiagen. Messenger RNAs from two different C8 populations were either labeled with CY3 or CY5, and then competitively hybridized to a UniGEM V5 human microarray by Incyte Genomics, Inc. (California). Detection and analysis of the ratio of transcript signals observed for the CY3 versus CY5 probes were performed by Incyte Genomics, Inc. As a result of these procedures, transcript levels for roughly 9,000 genes (or ESTs) were compared in C8 cells expressing either R3 or an out-of-frame (OF) R3, maintained in CBI.

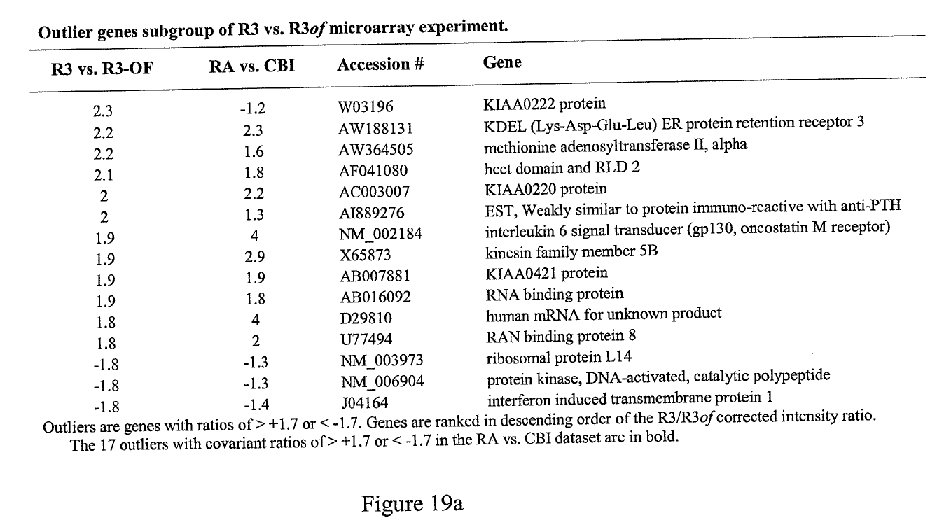

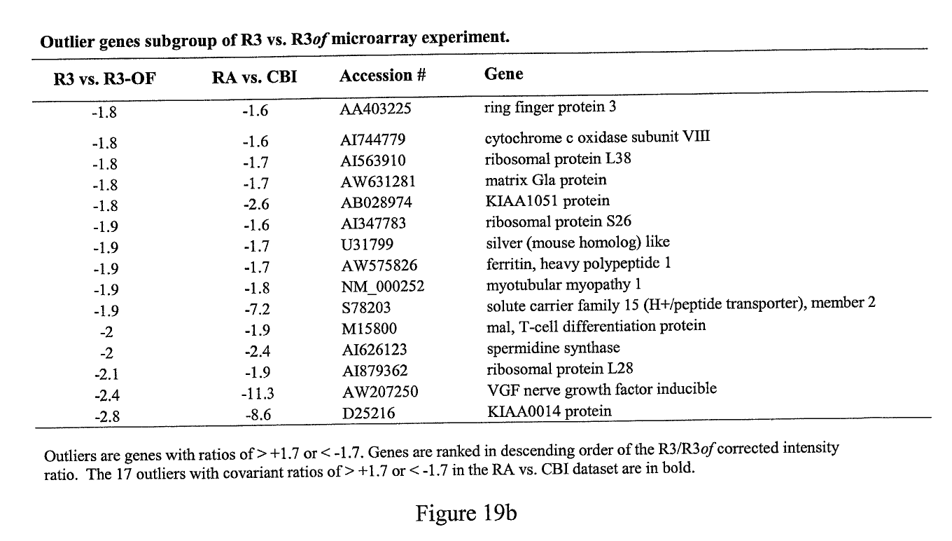

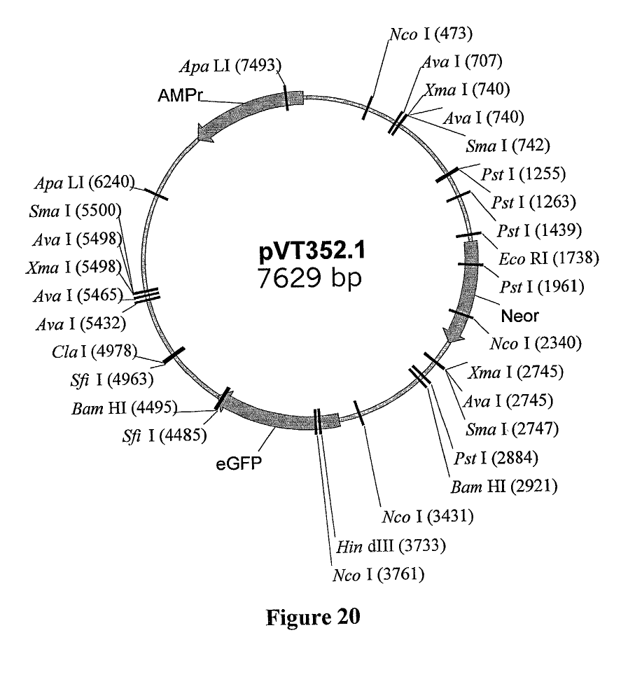

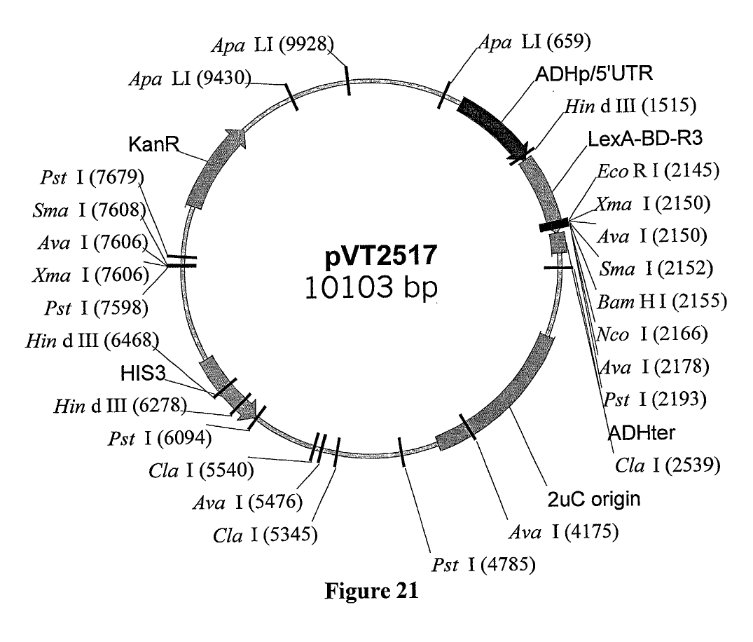

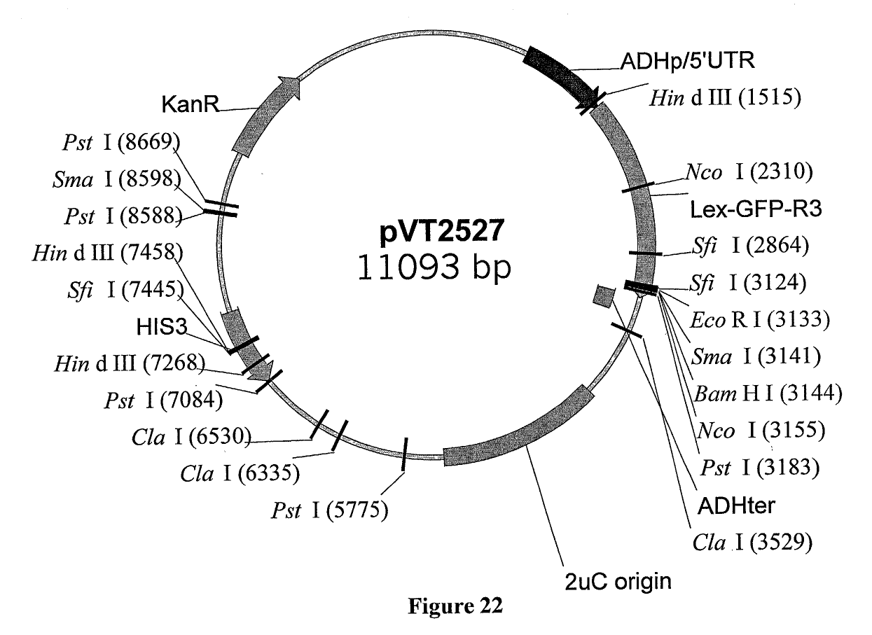

Analysis of the data showed that the expression profile of only 30 genes were altered by 1.8-fold or more with the largest difference being 2.8-fold (FIG. 19). These data suggested that the R3 perturbagen causes a specific, limited phenotypic change in the global expression profile of these cells. In contrast, RA treatment of C8 cells expressing R3-OF caused a more dramatic shift in molecular phenotype, with 285 genes altered in expression level by 1.8-fold or greater (data not shown). Notably, the subset of modulated genes in R3-expressing cells was a subset of the off-diagonal outliers in RA-treated cells. Seventeen of the 30 outliers in the R3 vs. R3-OF data were also outliers of the same sign in the RA experiment. In total, 29 of the 30 data points were positively correlated, and covariance calculations revealed that this correlation between the R3 subgroup and the equivalent genes in RA-treated cells was highly significant (covariance=1.89, p <<0.001; FIG. 7C). The R3 outlier subgroup was uncorrelated with an unrelated gene expression dataset (+/− cAMP treatment; covariance=0.07, p˜0.3) demonstrating that its covariance was restricted to the RA response (data not shown).

The above examples are provided to illustrate the invention but not to limit its scope. Other variants of the invention will be readily apparent to one of ordinary skill in the art and encompassed by the appended claims. All publications, patents and patent applications cited herein are hereby incorporated by reference.

What is claimed is:

1. An isolated polypeptide having RA pathway activity comprising a polypeptide sequence selected from the group consisting of:
(a) the polypeptide sequence of Perturbagen R3 (FIG. 15(b));
(b) the polypeptide sequence of Perturbagen F802 (FIG. 13(a));
(c) the polypeptide sequence of Perturbagen F820 (FIG. 13(b));
(d) biologically active modifications of (a), (b) or (c); and
(e) biologically active fragments of (a), (b) or (c).

2. The isolated polypeptide of claim 1 wherein said isolated polypeptide is (a) (b) or (c).

3. The isolated polypeptide of claim 1 consisting essentially of the sequence of Perturbagen R3 (FIG. 15(b)).

4. The isolated polypeptide of claim 3 wherein said isolated polypeptide comprises the amino acid sequence of Perturbagen R3 (FIG. 15(b)) except for one or more conservative amino acid substitutions.

5. The isolated polypeptide of claim 2 consisting of Perturbagen R3 (FIG. 15(b)).

6. The isolated polypeptide of claim 1 wherein said isolated polypeptide comprises a sequence at least 99% identical to the amino acid sequence of Perturbagen R3 (FIG. 15(b)).

7. The isolated polypeptide of claim 1 wherein said isolated polypeptide comprises a sequence at least 95% identical to the amino acid sequence of Perturbagen R3 (FIG. 15(b)).

8. The isolated polypeptide of claim 1 wherein said isolated polypeptide comprises a sequence at least 90% identical to the amino acid sequence of Perturbagen R3 (FIG. 15(b)).

9. The isolated polypeptide of claim 1 wherein said isolated polypeptide comprises a sequence at least 85% identical to the amino acid sequence of Perturbagen R3 (FIG. 15(b)).

10. The isolated polypeptide of claim 1 wherein said isolated polypeptide comprises a sequence at least 80% identical to the amino acid sequence of Perturbagen R3 (FIG. 15(b)).

11. The isolated polypeptide of claim 1 wherein said isolated polypeptide comprises a biologically active fragment of Perturbagen R3 (FIG. 15(b)) displaying a shift in RA-correlated reporter expression.

12. The isolated polypeptide of claim 1 wherein said isolated polypeptide is a closely related analog of Perturbagen R3 (FIG. 15(b)) wherein said analog displays biological activity of a shift in RA-correlated reporter expression.

13. The isolated polypeptide of claim 1 wherein said isolated polypeptide is an antigenic analog of Perturbagen R3 (FIG. 15(b)) wherein said analog binds to an antibody specific for the polypeptide of Perturbagen R3 (FIG. 15(b)).

14. The isolated polypeptide of claim 1 wherein said isolated polypeptide is an N-terminal fragment of Perturbagen R3 (FIG. 15(b)).

15. The isolated polypeptide of claim 14 wherein said N-terminal fragment comprises at least 10 amino acids of Perturbagen R3 (FIG. 15(b)).

16. The isolated polypeptide of claim 1 wherein said isolated polypeptide is a C-terminal fragment of Perturbagen R3 (FIG. 15(b)).

17. The isolated polypeptide of claim 16 wherein said C-terminal fragment comprises at least 10 amino acids of Perturbagen R3 (FIG. 15(b)).

18. The isolated polypeptide of claim 1 consisting essentially of the sequence of Perturbagen F802 (FIG. 13(a)).

19. The isolated polypeptide of claim 3 wherein said isolated polypeptide comprises the amino acid sequence of Perturbagen F802 (FIG. 13(a)) except for one or more conservative amino acid substitutions.

20. The isolated polypeptide of claim 2 consisting of Perturbagen F802 (FIG. 13(a)).

21. The isolated polypeptide of claim 1 wherein said isolated polypeptide comprises a sequence at least 99% identical to the amino acid sequence of Perturbagen F802 (FIG. 13(a)).

22. The isolated polypeptide of claim 1 wherein said isolated polypeptide comprises a sequence at least 95% identical to the amino acid sequence of Perturbagen F802 (FIG. 13(a)).

23. The isolated polypeptide of claim 1 wherein said isolated polypeptide comprises a sequence at least 90% identical to the amino acid sequence of Perturbagen F802 (FIG. 13(a)).

24. The isolated polypeptide of claim 1 wherein said isolated polypeptide comprises a sequence at least 85% identical to the amino acid sequence of Perturbagen F802 (FIG. 13(a)).

25. The isolated polypeptide of claim 1 wherein said isolated polypeptide comprises a sequence at least 80% identical to the amino acid sequence of Perturbagen F802 (FIG. 13(a)).

26. The isolated polypeptide of claim 1 wherein said isolated polypeptide comprises a biologically active fragment of Perturbagen F802 (FIG. 13(a)) displaying a shift in RA-correlated reporter expression.

27. The isolated polypeptide of claim 1 wherein said isolated polypeptide is a closely related analog of Perturbagen F802 (FIG. 13(a)) wherein said analog displays biological activity of a shift in RA-correlated reporter expression.

28. The isolated polypeptide of claim 1 wherein said isolated polypeptide is an antigenic analog of Perturbagen F802 (FIG. 13(a)) wherein said analog binds to an antibody specific for the polypeptide of Perturbagen F802 (FIG. 13(a)).

29. The isolated polypeptide of claim 1 wherein said isolated polypeptide is an N-terminal fragment of Perturbagen F802 (FIG. 13(a)).

30. The isolated polypeptide of claim 29 wherein said N-terminal fragment comprises at least 10 amino acids of Perturbagen F802 (FIG. 13(a)).

31. The isolated polypeptide of claim 1 wherein said isolated polypeptide is a C-terminal fragment of Perturbagen F802 (FIG. 13(a)).

32. The isolated polypeptide of claim 31 wherein said C-terminal fragment comprises at least 10 amino acids of Perturbagen F802 (FIG. 13(a)).

33. The isolated polypeptide of claim 1 consisting essentially of the sequence of Perturbagen F820 (FIG. 13(b)).

34. The isolated polypeptide of claim 3 wherein said isolated polypeptide comprises the amino acid sequence of Perturbagen F820 (FIG. 13(b)) except for one or more conservative amino acid substitutions.

35. The isolated polypeptide of claim 2 consisting of Perturbagen F820 (FIG. 13(b)).

36. The isolated polypeptide of claim 1 wherein said isolated polypeptide comprises a sequence at least 99% identical to the amino acid sequence of Perturbagen F820 (FIG. 13(b)).

37. The isolated polypeptide of claim 1 wherein said isolated polypeptide comprises a sequence at least 95% identical to the amino acid sequence of Perturbagen F820 (FIG. 13(b)).

38. The isolated polypeptide of claim 1 wherein said isolated polypeptide comprises a sequence at least 90% identical to the amino acid sequence of Perturbagen F820 (FIG. 13(b)).

39. The isolated polypeptide of claim 1 wherein said isolated polypeptide comprises a sequence at least 85% identical to the amino acid sequence of Perturbagen F820 (FIG. 13(b)).

40. The isolated polypeptide of claim 1 wherein said isolated polypeptide comprises a sequence at least 80% identical to the amino acid sequence of Perturbagen F820 (FIG. 13(b)).

41. The isolated polypeptide of claim 1 wherein said isolated polypeptide comprises a biologically active fragment of sequence Perturbagen F820 (FIG. 13(b)) displaying a shift in RA-correlated reporter expression.

42. The isolated polypeptide of claim 1 wherein said isolated polypeptide is a closely related analog of Perturbagen F820 (FIG. 13(b)) wherein said analog displays biological activity of a shift in RA-correlated reporter expression.

43. The isolated polypeptide of claim 1 wherein said isolated polypeptide is an antigenic analog of Perturbagen F820 (FIG. 13(b)) wherein said analog binds to an antibody specific for the polypeptide of Perturbagen F820 (FIG. 13(b)).

44. The isolated polypeptide of claim 1 wherein said isolated polypeptide is an N-terminal fragment of Perturbagen F820 (FIG. 13(b)).

45. The isolated polypeptide of claim 44 wherein said N-terminal fragment comprises at least 10 amino acids of Perturbagen F820 (FIG. 13(b)).

46. The isolated polypeptide of claim 1 wherein said isolated polypeptide is a C-terminal fragment of Perturbagen F820 (FIG. 13(b)).

47. The isolated polypeptide of claim 46 wherein said C-terminal fragment comprises at least 10 amino acids of Perturbagen F820 (FIG. 13(b)).

48. The polypeptide of claim 1 wherein said polypeptide is fused to heterologous sequence.

49. The polypeptide of claim 48 wherein said heterologous sequence is a scaffold.

50. The polypeptide of claim 49 wherein said scaffold is a fluorescent protein.

51. The polypeptide of claim 1 wherein said polypeptide is chemically modified.

52. The polypeptide of claim 51 wherein said polypeptide is radio labeled.

53. The polypeptide of claim 51 wherein said modification is selected from the group consisting of acetylation, glycosylation, or fluorescent tagging.

54. The polypeptide of claim 1 wherein said polypeptide is chemically synthesized.

55. An isolated polynucleotide encoding a polypeptide of claim 1.

56. The isolated polynucleotide of claim 55, wherein said polypeptide encodes sequences (a) (b) or (c).

57. An isolated polynucleotide encoding a polypeptide of claim 3, 18 or 33.

58. An isolated polynucleotide encoding a polypeptide of claim 4, 19 or 34.

59. An isolated polynucleotide encoding a polypeptide of claim 5, 20 or 35.

60. An isolated polynucleotide encoding a polypeptide of claim 6, 21 or 36.

61. An isolated polynucleotide encoding a polypeptide of claim 7, 22 or 37.

62. An isolated polynucleotide encoding a polypeptide of claim 8, 23 or 38.

63. An isolated polynucleotide encoding a polypeptide of claim 9, 24 or 39.

64. An isolated polynucleotide encoding a polypeptide of claim 10, 25 or 40.

65. An isolated polynucleotide encoding a polypeptide of claim 14, 29 or 44.

66. An isolated polynucleotide encoding a polypeptide of claim 16, 31 or 46.

67. An isolated polynucleotide comprising the DNA sequence selected from a group consisting of:
(a) Perturbagen R3 (FIG. 15(b));
(b) Perturbagen F802 (FIG. 13(a)); and
(c) Perturbagen F820 (FIG. 13(b)).

68. An isolated polynucleotide of claim 67 wherein said isolated polynucleotide is (a).

69. An isolated polynucleotide of claim 67 wherein said isolated polynucleotide is (b).

70. An isolated polynucleotide of claim 67 wherein said isolated polynucleotide is (c).

71. An isolated polynucleotide consisting essentially of the sequence of Perturbagen R3 (FIG. 15(b)).

72. An isolated polynucleotide consisting essentially of the sequence of Perturbagen F802 (FIG. 13(a)).

73. An isolated polynucleotide consisting essentially of the sequence of Perturbagen F820 (FIG. 13(b)).

74. The isolated polynucleotide of any one of claims 68, 69 or 70 wherein said isolated polynucleotide comprises a sequence at least 99% identical to said polynucleotide.

75. The isolated polynucleotide of any one of claims 68, 69 or 70 wherein said isolated polynucleotide comprises a sequence at least 95% identical to said polynucleotide.

76. The isolated polynucleotide of any one of claims 68, 69 or 70 wherein said isolated polynucleotide comprises a sequence at least 90% identical to said polynucleotide.

77. The isolated polynucleotide of any one of claims 68, 69 or 70 wherein said isolated polynucleotide comprises a sequence at least 85% identical to said polynucleotide.

78. The isolated polynucleotide of any one of claims 68, 69 or 70 wherein said isolated polynucleotide comprises a sequence at least 80% identical to said polynucleotide.

79. A vector comprising the polynucleotide of any one of claims 55, 56, 67, 71, 72 or 73.

80. The vector of claim 79, wherein said vector provides inducible expression.

81. A gene therapy vector comprising the polynucleotide of claims 55, 56, 67, 71, 72 or 73.

82. A host cell comprising the vector of claim 79.

83. A polynucleotide that hybridizes under stringent conditions to the polynucleotide of any one of claims 55, 56, 67, 71, 72 or 73.

84. A method for producing a RA pathway related polypeptide comprising culturing a population of host cells of claim 82 under conditions suitable for the expression of an encoded polypeptide and recovering expressed polypeptide from the host cell culture.

85. A composition comprising the polypeptide of claims 1, 2, 3, 18 or 33 in a pharmaceutically acceptable carrier.

86. An antibody to the polypeptide of claims 1, 2, 3, 18 or 33.

87. A method of identifying a cellular target that interacts with a RA pathway related polypeptide, comprising the steps of exposing a polypeptide of claim 1 to putative target molecules and identifying a polypeptide/target interaction pair.

88. The method of claim 87 wherein said step of exposing is performed in vitro and said step of identifying comprises detecting reporter expression, wherein said reporter expression is operatively linked to the formation of said interaction pair.

89. The method of claim 88 wherein said method is a yeast two-hybrid assay.

90. A method of screening for putative RA-related therapeutics, comprising the steps of:
a) exposing a polypeptide/target interaction pair obtained by the method of claim 87 to a plurality of agents; and
b) recovering a subpopulation of disrupting agents which competitively displace said polypeptide from said target; wherein said disrupting agents are putative RA-related therapeutics.

91. The method of claim 90, wherein said plurality of agents is a combinatorial chemical library.

92. A method of treating an RA pathway related condition, comprising the step of administering a therapeutically effective amount of the polypeptide of claim 1, or a pharmaceutically acceptable salt thereof.

93. An isolated RA pathway polypeptide comprising the PAT1 polypeptide (FIG. 16(d).

94. An isolated RA pathway polypeptide consisting essentially of the PAT1 polypeptide (FIG. 16(d)).

95. The isolated polypeptide of claim 94 wherein said isolated polypeptide comprises the amino acid sequence of the PAT1 polypeptide (FIG. 16(d)) except for one or more conservative amino acid substitutions.

96. The isolated RA pathway polypeptide consisting of the PAT1 polypeptide (FIG. 16(d)).

97. The isolated polypeptide of claim 94 wherein said isolated polypeptide comprises a sequence at least 99% identical to the amino acid sequence of the PAT1 polypeptide (FIG. 16(d)).

98. The isolated polypeptide of claim 94 wherein said isolated polypeptide comprises a sequence at least 95% identical to the amino acid sequence of the PAT1 polypeptide (FIG. 16(d)).

99. The isolated polypeptide of claim 94 wherein said isolated polypeptide comprises a sequence at least 90% identical to the amino acid sequence of the PAT1 polypeptide (FIG. 16(d)).

100. The isolated polypeptide of claim 94 wherein said isolated polypeptide comprises a sequence at least 85% identical to the amino acid sequence of the PAT1 polypeptide (FIG. 16(d)).

101. The isolated polypeptide of claim 94 wherein said isolated polypeptide comprises a sequence at least 80% identical to the amino acid sequence of the PAT1 polypeptide (FIG. 16(d)).

102. An isolated polynucleotide encoding the polypeptide of claims 93, 94 or 96.

103. An isolated polynucleotide encoding the PAT1 polypeptide (FIG. 16(d)).

104. An isolated polynucleotide comprising the DNA sequence of PAT1 (FIG. 16 (c)).

105. An isolated polynucleotide consisting essentially of the DNA sequence of PAT1 (FIG. 16(c)).

106. An isolated polynucleotide consisting of the DNA sequence of PAT1 (FIG. 16 (c)).

107. A vector comprising the polynucleotide of claim 102.

108. A gene therapy vector comprising the polynucleotide of claim 102.

109. A host cell comprising the polynucleotide of claim 102.

110. A polynucleotide that hybridizes under stringent conditions to the polynucleotide of claim 102.
